(19) United States
(12) Patent Application Publication
(10) Pub. No.: US 2002/0102568 A1
(43) Pub. Date: Aug. 1, 2002

(54) Nucleic acid sensor molecules

(76) Inventors: Nassim Usman, Lafayette, CO (US); James A. McSwiggen, Boulder, CO (US); Shawn Zinnen, Denver, CO (US); Scott Seiwert, Lyons, CO (US); Peter Haeberli, Berthoud, CO (US); Bharat Chowrira, Broomfield, CO (US); Lawrence Blatt, Boulder, CO (US); Narendra K. Vaish, Boulder, CO (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 09/877,526

(22) Filed: Jun. 8, 2001

Related U.S. Application Data
(60) Provisional application No. 60/187,128, filed on Mar. 6, 2000.

(30) Foreign Application Priority Data
Mar. 6, 2001 (US) PCT/US01/07163

(51) Int. Cl. C12Q 1/68; C07H 21/04

(52) U.S. Cl. 435/6; 536/24.3

(57) ABSTRACT

Nucleic acid sensor molecules and methods are disclosed for the detection and amplification of signaling agents using enzymatic nucleic acid constructs, including hammerhead enzymatic nucleic acid molecules, inozymes, G-cleaver enzymatic nucleic acid molecules, zinzymes, amberzymes and DNAzymes; kits for detection and amplification; use in diagnostics, nucleic acid circuits, nucleic acid computers, therapeutics, target validation, target discovery, drug optimization, SNP detection, SNP scoring, proteome scoring and other uses are disclosed.

This patent application is a continuation-in-part of Usman et al., U.S. Ser. No.______, filed Mar. 6, 2001, entitled “NUCLEIC ACID SENSOR MOLECULES”, which claims priority from Usman et al., U.S. Ser. No. (60/187,128), filed March 6, 2000, entitled “A PROCESS FOR THE DETECTION OF NUCLEIC ACID USING NUCLEIC ACID CATALYSTS”. These applications are hereby incorporated by reference herein in their entirety including the drawings.

### FIELD OF THE INVENTION

This invention relates to novel molecular sensors that utilize enzymatic nucleic acid constructs whose activity can be modulated by the presence or absence of various signaling agents. The present invention further relates to the use of the enzymatic nucleic acid constructs as molecular sensors capable of modulating the activity, function, or physical properties of other molecules. The invention also relates to the use of the enzymatic nucleic acid constructs as a diagnostic application, useful in identifying signaling agents in a variety of applications, for example, in clinical, industrial, environmental, agricultural and/or research settings. The invention further relates to the use of the nucleic acid sensor constructs as a tool to identify the presence of genes and/or gene products which are indicative of a particular genotype and/or phenotype, for example a disease state, infection, or related condition within patients. In addition, the invention relates to the use of nucleic acid sensor molecules in nucleic acid-based electronics, including nucleic acid-based circuits and computers.

### BACKGROUND OF THE INVENTION

The following is a brief description of diagnostic and sensor-based applications for nucleic acids. This summary is provided only for understanding of the invention that follows. This summary is not an admission that all of the work described below is prior art to the claimed invention.

The detection of biomolecules, for example nucleic acids, can be highly beneficial in the diagnosis of diseases or medical disorders. By determining the presence of a specific nucleic acid sequence, investigators can confirm the presence of a virus, bacterium, genetic mutation, and other conditions that can relate to a disease. Assays for nucleic acid sequences can range from simple methods for detection, such as northern blot hybridization using a radiolabeled or fluorescent probe to detect the presence of a nucleic acid molecule, to the use of polymerase chain reaction (PCR) to amplify a small quantity of a specific nucleic acid to the point at which it can be used for detection of the sequence by hybridization techniques. The polymerase chain reaction, uses DNA polymerases to logarithmically amplify the desired sequence (U.S. Pat. Nos. 4,683,195; 4,683,202) using prefabricated primers to locate specific sequences. Nucleotide probes can be labeled using dyes, fluorescent, chemiluminescent, radioactive, or enzymatic labels which are commercially available. These probes can be used to detect by hybridization, the expression of a gene or related sequences in cells or tissue samples in which the gene is a normal component, as well as to screen sera or tissue samples from humans suspected of having a disorder arising from infection with an organism, or to detect novel or altered genes as might be found in tumorigenic cells. Nucleic acid primers can also be prepared which, with reverse transcriptase or DNA polymerase and PCR, can be used for detection of nucleic acid molecules that are present in very small amounts in tissues or fluids.

PCR utilizes protein enzymes (DNA polymerase) to detect specific nucleotide sequences. PCR has several disadvantages, for example requiring a high degree of technical competence for reliability, high reagent costs, and sensitivity to contamination resulting in false positives.

Several groups to date have completed draft sequences of the entire human genome. To capitalize on this information, an effort to correlate changes in specific mRNA levels with different disease states has been initiated. The synergy of these efforts has been highly successful and there is now a wealth of information relating specific changes in gene expression to disease states. One drawback to the currently available data is that it is not always true that a disease state is reflected by changes in the level of gene expression. Increasingly, post-translational events that control the function of gene products (such as protein processing and protein phosphorylation) have been shown to play important roles in the conversion from a “well” to “diseased” phenotype. Thus, to efficiently use the data generated in the human genome project for the benefit of human health, a profile of disease-specific genomes and proteomes must be generated. Such information will be essential for the generation of treatment outcomes data that link patient and disease characteristics with future treatment events. Therefore, a clear need exists for molecular tools that can generate such disease specific genomes and proteomes or Diagnostic Molecular Profiles that correlate individual cellular and molecular events with disease outcomes profiles. These profiles can then be used to rationally drive treatment policy decisions resulting in better patient care and reductions in health care spending.

A class of enzymes which can been utilized for diagnostic and sensor purposes are enzymatic nucleic acid molecules (Kuwabara et al., 2000, Curr. Opin. Chem. Bio., 4, 669; Porta et al., 1995, Biochemistry, 13, 161; Soukup et al., 1999, TIBTECH, 17, 469; Marshall et al., 1999, Nature Struc Biol., 6, 992). The enzymatic nature of an enzymatic nucleic acid molecule can be advantageous over other sensor technologies, since the concentration of analyte necessary to generate a detectable response can be lower than that required with other sensor systems which can require amplification steps. This advantage reflects the ability of the enzymatic nucleic acid molecule to act enzymatically. Thus, a specific enzymatic nucleic acid molecule is able to amplify a given signal in response to a single recognition event. Such enzymatic nucleic acid-based sensor molecules are often referred to in the art as allosteric ribozymes or allosteric DNAzymes.

In addition, the enzymatic nucleic acid molecule is a highly specific sensor molecule that can be engineered to respond to a variety of different signaling events. The use of in vitro selection techniques can be applied to the selection of new enzymatic nucleic acid molecules that are capable of allosteric modulation. Previous work in this area has focused on combining known aptamer and enzymatic nucleic acid molecule sequences (Breaker, International PCT Publication No. WO 98/2714). Later work has revealed bridge sequences that connect the receptor and enzymatic sequence domains together. These bridging sequences function such that binding of a ligand to the receptor domain triggers a conformational change within the bridge, thus modulating phosphodiester cleavage activity of the adjoining enzymatic sequence (Breaker, International PCT Publication No. WO 00/26226).

George et al., U.S. Pat. Nos. 5,834,186 and 5,741,679, describe regulatable RNA molecules whose activity is altered in the presence of a ligand.

Shih et al., U.S. Pat. No. 5,589,332, describe a method for the use of ribozymes to detect macromolecules such as proteins and nucleic acid.

Nathan et al., U.S. Pat. No. 5,871,914, describe a method for detecting the presence of an assayed nucleic acid based on a two component ribozyme system containing a detection ensemble and an RNA amplification ensemble.

Nathan and Ellington, International PCT publication No. WO 00/24931, describe the detection of an analyte by a catalytic nucleic acid sequence which converts a nucleic acid substrate to a catalytic nucleic acid product in the presence of the analyte. The catalytic nucleic acid product is then amplified, by PCR.

Sullenger et al., International PCT publication No. WO 99/29842, describe nucleic acid mediated RNA tagging and RNA revision.

### SUMMARY OF THE INVENTION

The present invention relates to nucleic acid-based molecular sensors whose activity can be modulated by the presence or absence of various signaling agents, ligands, and/or target signaling molecules. The invention further relates to a method for the detection of specific target signaling molecules such as nucleic acid molecules, proteins, peptides, antibodies, polysaccharides, lipids, sugars, metals, microbial or cellular metabolites, analytes, pharmaceuticals, and other organic and inorganic molecules using nucleic acid sensor molecules in a variety of analytical settings, including clinical, industrial, veterinary, genomics, environmental, and agricultural applications. The invention further relates to the use of the nucleic acid sensor molecule as molecular sensors capable of modulating the activity, function, or physical properties of other molecules. The present invention also contemplates the use of the nucleic acid sensor molecule constructs as molecular switches, capable of inducing or negating a response in a system, for example in a nucleic acid-based circuit or computer.

The invention further relates to the use of nucleic acid sensor molecules in a diagnostic application to identify the presence of a target signaling molecule such as a gene and/or gene products which are indicative of a particular genotype and/or phenotype, for example, a disease state, infection, or related condition within patients or patient samples. The invention also relates to a method for the diagnosis of disease states or physiological abnormalities related to the expression of viral, bacterial or cellular RNA and DNA.

Diagnostic applications of the nucleic acid sensor molecules include the use of the nucleic acid sensor molecules for prospective diagnosis of disease, prognosis of therapeutic effect and/or dosing of a drug or class of drugs, prognosis and monitoring of disease outcome, monitoring of patient progress as a function of an approved drug or a drug under development, patient surveillance and screening for drug and/or drug treatment. Diagnostic applications include the use of nucleic acid sensors for research, development and commercialization of products for the rapid detection of macromolecules, such as mammalian viral nucleic acids, prions and viroids for the diagnosis of diseases associated with viruses, prions and viroids in humans and animals.

Nucleic acid sensor molecules can also be used in assays to assess the specificity, toxicity and effectiveness of various small molecules, nucleoside analogs, or non-nucleic acid drugs, or doses of a specific small molecules, nucleoside analogs or nucleic acid and non-nucleic acid drugs, against validated targets or biochemical pathways and include the use of nucleic acid sensors in assays involved in high-throughput screening, biochemical assays, including cellular assays, in vivo animal models, clinical trial management, and for mechanistic studies in human clinical studies. The nucleic acid sensor can also be used for the detection of pathogens, biochemicals, for example proteins, organic compounds, or inorganic compounds, in humans, plants, animals or samples therefrom, in connection with environmental testing or detection of biohazards. The use of the nucleic acid sensor molecules in other applications such a functional genomics, target validation and discovery, agriculture or diagnostics, for example the diagnosis of disease, or the prevention or treatment of human or animal disease is also contemplated.

In one embodiment, the invention features a nucleic acid sensor molecule comprising an enzymatic nucleic acid component and one or more sensor components, wherein, in response to an interaction of a target signaling molecule with the nucleic acid sensor molecule, the enzymatic nucleic acid component catalyzes a chemical reaction involving covalent attachment of at least a portion of a reporter molecule to the nucleic acid sensor molecule.

In another embodiment, the invention features a method, comprising the steps of: (a) contacting a nucleic acid sensor molecule comprising an enzymatic nucleic acid component and one or more sensor components, in which the enzymatic nucleic acid component catalyzes a chemical reaction in response to an interaction between a target signaling molecule and the nucleic acid sensor molecule, with a system under conditions suitable for the enzymatic nucleic acid component to catalyze a chemical reaction involving the attachment of at least a portion of a reporter molecule to the nucleic acid sensor molecule in the presence of a target signaling agent; and (b) assaying for the attachment of the reporter molecule to the nucleic acid sensor molecule.

In another embodiment, the invention features a nucleic acid sensor molecule comprising an enzymatic nucleic acid component and one or more sensor components, wherein, in response to an interaction of a target signaling molecule with the nucleic acid sensor molecule, the enzymatic nucleic acid component can carry out a chemical reaction involving isomerization of at least a portion of a reporter molecule.

In another embodiment, the invention features a method, comprising the steps of: (a) contacting a nucleic acid sensor molecule comprising an enzymatic nucleic acid component and one or more sensor components, wherein in response to an interaction of a target signaling molecule with the nucleic acid sensor molecule, the enzymatic nucleic acid component can carry out a chemical reaction involving isomerization of a reporter molecule, with a system under conditions suitable for the enzymatic nucleic acid component of the nucleic acid sensor molecule to isomerize of at least a portion of the reporter molecule in the presence of a target signaling agent; and (b) assaying for the isomerization reaction.

In another embodiment, the invention features a nucleic acid sensor molecule comprising an enzymatic nucleic acid component and one or more sensor components, wherein in response to an interaction of a target signaling molecule with the nucleic acid sensor molecule, the enzymatic component catalyses a chemical reaction on a non-oligonucleotide-based portion of a reporter molecule. For example, the chemical reaction catalyzed by the enzymatic compound can be a phosphorylation or dephosphorylation reaction.

In another embodiment, the invention features a method comprising the steps of: (a) contacting a nucleic acid sensor molecule comprising an enzymatic nucleic acid component and one or more sensor components, in which the enzymatic component catalyses a chemical reaction involving phosphorylation of a non-oligonucleotide-based portion of a reporter molecule in response to an interaction of a target signaling molecule with the nucleic acid sensor molecule, with a system under conditions suitable for the enzymatic nucleic acid component to phosphorylate a component of the reporter molecule in the presence of a target signaling agent; and (b) assaying for the phosphorylation reaction.

In another embodiment, the invention features a method comprising the steps of: (a) contacting a nucleic acid sensor molecule comprising an enzymatic nucleic acid component and one or more sensor components, in which the enzymatic component catalyses a chemical reaction involving dephosphorylation of a non-oligonucleotide-based portion of a reporter molecule in response to an interaction of a target signaling molecule with the nucleic acid sensor molecule, with a system under conditions suitable for the enzymatic nucleic acid component to dephosphorylate a component of the reporter molecule in the presence of a target signaling agent; and (b) assaying for the dephosphorylation reaction.

In one embodiment, the nucleic acid sensor molecule of the instant invention features an enzymatic component and a sensor component that are distinct moieties.

In another embodiment, the nucleic acid sensor molecule of the instant invention features a linker region that joins a sensor component to an enzymatic nucleic acid component.

In another embodiment, the invention features a nucleic acid sensor molecule comprising an enzymatic nucleic acid component and one or more sensor components, wherein, in response to an interaction of a target signaling molecule with the nucleic acid sensor molecule, the enzymatic nucleic acid component catalyzes a chemical reaction involving covalent attachment of at least a portion of a reporter molecule to at least a portion of the nucleic acid sensor molecule, wherein the reporter molecule comprises the formula:

R₁—L—R₂

wherein R1 is selected from the group consisting of alkyl, alkoxy, hydrogen, hydroxy, sulfhydryl, ester, anhydride, acid halide, amide, nitrile, phosphate, phosphonate, nucleoside, nucleotide, oligonucleotide; R2 is selected from the group consisting of molecular beacons, small molecules, fluorophores, chemophores, ionophores, radio-isotopes, photophores, peptides, proteins, enzymes, antibodies, nucleic acids, and enzymatic nucleic acids; L represents a linker which can be present or absent, and “—” represents a chemical bond.

In another embodiment, the invention features a nucleic acid sensor molecule comprising an enzymatic nucleic acid component and one or more sensor components, wherein, in response to an interaction of a target signaling molecule with the nucleic acid sensor molecule, the enzymatic nucleic acid component can carry out a chemical reaction involving isomerization of at least a portion of a reporter molecule, wherein the reporter molecule comprises the formula:

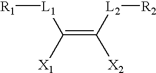

wherein R1 and R2 each represent compounds, which can be the same or different, that generate a detectable signal or quench a detectable signal when an isomerization reaction is catalyzed, selected from the group consisting of molecular beacons, small molecules, fluorophores, chemophores, ionophores, radio-isotopes, photophores, peptides, proteins, enzymes, antibodies, nucleic acids, and enzymatic nucleic acids; L1 and L2 each represent a linker which can be the same or different and which can be present or absent; X1 and X2 each represent an atom, compound, or molecule that can be the same or different, and “—” represents a chemical bond.

In another embodiment, the invention features a nucleic acid sensor molecule comprising an enzymatic nucleic acid component and one or more sensor components, wherein, in response to an interaction of a target signaling molecule with the nucleic acid sensor molecule, the enzymatic nucleic acid component can carry out a chemical reaction involving isomerization of at least a portion of a reporter molecule, wherein the reporter molecule comprises the formula:

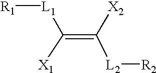

wherein R1 and R2 each represent compounds, which can be the same or different, that generate a detectable signal or quench a detectable signal when an isomerization reaction is catalyzed, selected from the group consisting of molecular beacons, small molecules, fluorophores, chemophores, ionophores, radio-isotopes, photophores, peptides, proteins, enzymes, antibodies, nucleic acids, and enzymatic nucleic acids; L1 and L2 each represent a linker which can be the same or different and which can be present or absent; X1 and X2 represent an atom, compound, or molecule that can be the same or different, and “—” represents a chemical bond.

In one embodiment, the detection of a chemical reaction catalyzed by a nucleic acid sensor molecule of the instant invention is indicative of the presence of the target signaling molecule in a system.

In another embodiment, the absence of a chemical reaction catalyzed by a nucleic acid sensor molecule of the instant invention is indicative of a system lacking the target signaling molecule.

In another embodiment, the invention features a method comprising the steps of: (a) contacting a nucleic acid sensor molecule which comprises (i) an enzymatic nucleic acid component comprising a substrate binding region and a catalytic region; and (ii) a sensor component comprising a nucleic acid sequence that upon interacting with a complementary sequence in the enzymatic nucleic acid component, inhibits the activity of the enzymatic nucleic acid component, and a reporter molecule comprising a nucleic acid sequence complementary to the substrate binding region of the enzymatic nucleic acid component of the nucleic acid sensor molecule, with a system under conditions suitable for the enzymatic nucleic acid component of the nucleic acid sensor molecule to catalyze cleavage of the reporter molecule in the presence of a target signaling molecule; and (b) assaying for the cleavage reaction of step (a).

In another embodiment, the invention features a method comprising the steps of: (a) contacting a nucleic acid sensor molecule which comprises (i) an enzymatic nucleic acid component comprising a substrate binding region and a catalytic region; and (ii) a sensor component comprising a nucleic acid sequence that when interacts with a complementary sequence in the enzymatic nucleic acid component inhibits the activity of the enzymatic nucleic acid component and a reporter molecule comprising a nucleic acid sequence complementary to the substrate binding region of the enzymatic nucleic acid component of the nucleic acid sensor molecule, with a system under conditions suitable for the enzymatic nucleic acid component of the nucleic acid sensor molecule to catalyze a ligation reaction involving the reporter molecule in the presence of a target signaling molecule, and (b) assaying for the ligation reaction in step (a).

In one embodiment of the inventive method, the ligation reaction catalyzed by the nucleic acid sensor molecule causes at least a portion of a reporter molecule to be attached to the nucleic acid sensor molecule.

In another embodiment, the ligation reaction catalyzed by the nucleic acid sensor molecule causes at least a portion of a reporter molecule to be attached to a separate molecule. Suitable molecules include, for example, a separate nucleic acid molecule, peptide, protein, small molecule, biotin, or surface.

Also, in one embodiment of the inventive method, the cleavage of a reporter molecule catalyzed by the nucleic acid sensor molecule is indicative of the presence of the target signaling molecule in the system. In another embodiment, the absence of cleavage of a reporter molecule catalyzed by the nucleic acid sensor molecule is indicative of the system lacking the target signaling molecule.

In another embodiment of the inventive method, the ligation of a reporter molecule catalyzed by the nucleic acid sensor molecule is indicative of the presence of the target signaling molecule in the system. In another embodiment, the absence of ligation of a reporter molecule catalyzed by the nucleic acid sensor molecule is indicative of the system lacking the target signaling molecule.

In one embodiment, the system of the instant invention is an in vitro system. Preferably, the in vitro system is a sample derived from the group consisting of a patient, plant, water, beverage, food preparation, and soil.

In one embodiment, the target signaling molecule of the instant invention is an RNA, DNA, analog of RNA or analog of DNA. Preferably, the target signaling molecule of the instant invention is an RNA derived from a bacteria, virus, fungi, plant or mammalian genome.

In one embodiment, the enzymatic nucleic acid component of the nucleic acid sensor molecule is selected from the group consisting of hammerhead, hairpin, inozyme, G-cleaver, Zinzyme, RNase P EGS nucleic acid and Amberzyme motif. In another embodiment, the enzymatic nucleic acid component of the nucleic acid sensor molecule is a DNAzyme.

In one embodiment, the reporter molecule of the instant invention comprises a detectable label selected from the group consisting of chromogenic substrate, fluorescent labels, chemiluminescent labels, and radioactive labels and enzymes. Suitable enzymes include, for example, luciferase, horseradish peroxidase, and alkaline phosphatase.

In another embodiment, the reporter molecule of the instant invention is immobilized on a solid support. Suitable solid supports include silicon-based chips, silicon-based beads, controlled pore glass, polystyrene, cross-linked polystyrene, nitrocellulose, biotin, plastics, metals and polyethylene films.

In one embodiment the sensor component of the nucleic acid sensor molecule is RNA, DNA, analog of RNA or analog of DNA.

In another embodiment, the sensor component of the nucleic acid sensor molecule is covalently linked to the nucleic acid sensor molecule by a linker. Suitable linkers include one or more nucleotides, abasic moieties, polyethers, polyamines, polyamides, peptides, carbohydrates, lipids, and polyhydrocarbon compounds, and any combination thereof

In another embodiment, the sensor component of the nucleic acid sensor molecule is not covalently linked to the nucleic acid sensor molecule.

In another embodiment, the reporter molecule of the instant invention is RNA, DNA, RNA analog, or DNA analog.

In another embodiment, the invention features a kit comprising: (a) a nucleic acid sensor molecule which comprises (i) an enzymatic nucleic acid component comprising a substrate binding region and a catalytic region; and (ii) a sensor component comprising a nucleic acid which interacts with a complementary sequence in the enzymatic nucleic acid component to inhibit the activity of the enzymatic nucleic acid component; and (b) a reporter molecule that can be modified, i.e., cleaved, ligated, polymerized, isomerized, phorphorylated, and/or dephosphorylated by the enzymatic nucleic acid component of the nucleic acid sensor molecule in the presence of a target signaling molecule, wherein the reporter molecule comprises a chemical moiety capable of emitting a detectable signal upon its modification.

In another embodiment, the invention features a kit which comprises: (a) a nucleic acid sensor molecule comprising an enzymatic nucleic acid component and one or more sensor components; and (b) a reporter molecule, wherein, in response to an interaction of a target signaling molecule with the nucleic acid sensor molecule, the enzymatic nucleic acid component catalyzes a chemical reaction involving covalent attachment of at least a portion of a reporter molecule to the nucleic acid sensor molecule.

In another embodiment, the invention features a kit which comprises: (a) a nucleic acid sensor molecule comprising, an enzymatic nucleic acid component and one or more sensor components; and (b) a reporter molecule, wherein in response to an interaction of a target signaling molecule with the nucleic acid sensor molecule, the enzymatic nucleic acid component is capable of carrying out a chemical reaction involving isomerization of at least a portion of a reporter molecule.

In another embodiment, the invention features a kit which comprises: (a) a nucleic acid sensor molecule comprising an enzymatic nucleic acid component and one or more sensor components; and (b) a reporter molecule having a non-oligonucleotide-based portion, wherein, in response to an interaction of a target signaling molecule with the nucleic acid sensor molecule, the enzymatic component catalyses a chemical reaction involving phosphorylation of a non-oligonucleotide-based portion of a reporter molecule.

In another embodiment, the invention features a kit which comprises: (a) a nucleic acid sensor molecule comprising an enzymatic nucleic acid component and one or more sensor components; and (b) a reporter molecule having a non-oligonucleotide-based portion, wherein, in response to an interaction of a target signaling molecule with the nucleic acid sensor molecule, the enzymatic component catalyses a chemical reaction involving dephosphorylation of a non-oligonucleotide-based portion of a reporter molecule.

In another embodiment, the invention features a method comprising the step of contacting one or more components of a kit of the instant invention with a system under conditions suitable for the reporter molecule in the kit to be cleaved by the nucleic acid sensor molecule in the kit in the presence of a target signaling molecule.

In another embodiment, the invention features a method comprising the step of contacting one or more components of a kit of the instant invention with a system under conditions suitable for at least a portion of the reporter molecule in the kit to be covalently attached to the nucleic acid sensor molecule in the kit in the presence of a target signaling molecule.

In another embodiment, the invention features a method comprising the step of contacting one or more components of a kit of the instant invention with a system under conditions suitable for at least a portion of the reporter molecule in the kit to be isomerized by the nucleic acid sensor molecule in the kit in the presence of a target signaling molecule.

In another embodiment, the invention features a method comprising the step of contacting one or more components of a kit of the instant invention with a system under conditions suitable for at least a portion of the reporter molecule in the kit to be phosphorylated by the nucleic acid sensor molecule in the kit in the presence of a target signaling molecule.

In another embodiment, the invention features a method comprising the step of contacting one or more components of a kit of the instant invention with a system under conditions suitable for at least a portion of the reporter molecule in the kit to be dephosphorylated by the nucleic acid sensor molecule in the kit in the presence of a target signaling molecule.

In another embodiment, the invention features a nucleic acid circuit including a nucleic acid sensor molecule comprising an enzymatic nucleic acid component and one or more sensor components, wherein, in response to an interaction of a target signaling agent with the nucleic acid sensor molecule, the enzymatic nucleic acid component catalyzes a chemical reaction involving ligation of at least a portion of a nucleic acid based component of the nucleic acid circuit.

In another embodiment, the invention features a nucleic acid circuit including a nucleic acid sensor molecule comprising an enzymatic nucleic acid component and one or more sensor components, wherein, in response to an interaction of a target signaling agent with the nucleic acid sensor molecule, the enzymatic nucleic acid component catalyzes a chemical reaction involving cleavage of at least a portion of a nucleic acid based component of the nucleic acid circuit.

In another embodiment, the invention features a nucleic acid computer comprising one or more nucleic acid circuits including a nucleic acid sensor molecule comprising an enzymatic nucleic acid component and one or more sensor components, wherein, in response to an interaction of a target signaling agent with the nucleic acid sensor molecule, the enzymatic nucleic acid component catalyzes a chemical reaction involving ligation of at least a portion of a nucleic acid based component of the nucleic acid circuit.

In another embodiment, the invention features a nucleic acid computer including one or more nucleic acid circuits having a nucleic acid sensor molecule comprising an enzymatic nucleic acid component and one or more sensor components, wherein, in response to an interaction of a target signaling agent with the nucleic acid sensor molecule, the enzymatic nucleic acid component catalyzes a chemical reaction involving cleavage of at least a portion of a nucleic acid based component of the nucleic acid circuit.

In one embodiment, the signaling agent of the instant invention is current. In another embodiment, the signaling agent of the instant invention is voltage. In yet another embodiment, the signaling agent of the instant invention is impedance.

In one embodiment, the nucleic acid computer of the instant invention comprises a plurality of nucleic acid circuits that are arranged in a parallel array. In one embodiment, the nucleic acid computer of the instant invention is used to detect a signaling agent. In another embodiment, the nucleic acid computer of the instant invention is used to solve a problem.

In one embodiment, the invention features a method comprising the steps of: (a) contacting a nucleic acid circuit of the invention with a signaling agent under conditions suitable for the nucleic acid sensor molecule to ligate at least a portion of a nucleic acid based component of the nucleic acid circuit; and (b) assaying for the ligation of step (a).

In another embodiment, the invention features a method comprising the steps of: (a) contacting the nucleic acid circuit of the invention with a signaling agent under conditions suitable for the nucleic acid sensor molecule to cleave at least a portion of a nucleic acid based component of the nucleic acid circuit; and (b) assaying for the cleavage of step (a).

In one embodiment, the ligation of a nucleic acid circuit by a nucleic acid sensor molecule of the invention is assayed by measuring parameters selected from the group consisting of current, voltage, capacitance, and impedence.

Also, in one embodiment, the cleavage of a nucleic acid circuit by a nucleic acid sensor molecule of the invention is assayed by measuring parameters selected from the group consisting of current, voltage, capacitance, and impedence.

In one embodiment, the invention features a method for isolating a nucleic acid sensor molecule of the instant invention, comprising the steps of: (a) contacting a random pool of nucleic acids with a target signaling molecule and a reporter molecule, and (b) isolating a nucleic acid sensor molecule that can catalyze a chemical reaction involving covalent attachment of at least a portion of the reporter molecule to the nucleic acid sensor molecule in the presence of the target signaling molecule.

In another embodiment, the invention features a method for isolating a nucleic acid sensor molecule of the instant invention comprising the steps of: (a) contacting a random pool of nucleic acids with a target signaling molecule and a reporter molecule, and (b) isolating a nucleic acid sensor molecule that can catalyze a chemical reaction involving ligation of at least a portion of the reporter molecule to the nucleic acid sensor molecule in the presence of the target signaling molecule.

In another embodiment, the invention features a method for isolating a nucleic acid sensor molecule of the instant invention comprising the steps of: (a) contacting a random pool of nucleic acids with a target signaling molecule and a non-oligonucleotide-based reporter molecule, and (b) isolating a nucleic acid sensor molecule that can catalyze a chemical reaction involving phosphorylation a non-oligonucleotide-based portion of the reporter molecule by the nucleic acid sensor molecule in the presence of the target signaling molecule.

In another embodiment, the invention features a method for isolating a nucleic acid sensor molecule of the instant invention, comprising the steps of: (a) contacting a random pool of nucleic acids with a target signaling molecule and a non-oligonucleotide-based reporter molecule, and (b) isolating a nucleic acid sensor molecule that can catalyze a chemical reaction involving dephosphorylation of a non-oligonucleotide-based portion of the reporter molecule by the nucleic acid sensor molecule in the presence of the target signaling molecule.

In one embodiment, the invention features a nucleic acid sensor molecule comprising an enzymatic nucleic acid component and one or more sensor components, wherein, in response to an interaction of a single stranded RNA (ssRNA) having a SNP with the nucleic acid sensor molecule in a system, the enzymatic nucleic acid component catalyzes a chemical reaction resulting in a detectable response.

In another embodiment, the invention features a nucleic acid sensor molecule comprising an enzymatic nucleic acid component and one or more sensor components, wherein, in response to an interaction of a single stranded DNA (ssDNA) having a SNP with the nucleic acid sensor molecule in a system, the enzymatic nucleic acid component catalyzes a chemical reaction resulting in a detectable response.

In yet another embodiment, the invention features a nucleic acid sensor molecule comprising an enzymatic nucleic acid component and one or more sensor components, wherein, in response to an interaction of a peptide with the nucleic acid sensor molecule in a system, the enzymatic nucleic acid component catalyzes a chemical reaction resulting in a detectable response.

In another embodiment, the invention features a nucleic acid sensor molecule comprising an enzymatic nucleic acid component and one or more sensor components, wherein, in response to an interaction of a protein with the nucleic acid sensor molecule in a system, the enzymatic nucleic acid component catalyzes a chemical reaction resulting in a detectable response.

In one embodiment, the invention features a nucleic acid sensor molecule comprising an enzymatic nucleic acid component and one or more sensor components, wherein, in response to an interaction of a single stranded RNA (ssRNA) with the nucleic acid sensor molecule in a system, the enzymatic nucleic acid component catalyzes a chemical reaction resulting in the cleavage of a predetermined RNA molecule associated with a disease.

In another embodiment, the invention features a nucleic acid sensor molecule comprising an enzymatic nucleic acid component and one or more sensor components, wherein, in response to an interaction of a single stranded DNA (ssDNA) with the nucleic acid sensor molecule in a system, the enzymatic nucleic acid component catalyzes a chemical reaction resulting in the cleavage of a predetermined RNA molecule associated with a disease.

In yet another embodiment, the invention features a nucleic acid sensor molecule comprising an enzymatic nucleic acid component and one or more sensor components, wherein, in response to an interaction of a peptide with the nucleic acid sensor molecule in a system, the enzymatic nucleic acid component catalyzes a chemical reaction resulting in the cleavage of a predetermined RNA molecule associated with a disease.

In another embodiment, the invention features a nucleic acid sensor molecule comprising an enzymatic nucleic acid component and one or more sensor components, wherein, in response to an interaction of a protein with the nucleic acid sensor molecule in a system, the enzymatic nucleic acid component catalyzes a chemical reaction resulting in the cleavage of a predetermined RNA molecule associated with a disease.

In one embodiment, the invention features a nucleic acid sensor molecule comprising an enzymatic nucleic acid component and one or more sensor components, wherein, in response to an interaction of a single stranded RNA (ssRNA) with the nucleic acid sensor molecule in a system, the enzymatic nucleic acid component catalyzes a chemical reaction resulting in ligation of a predetermined RNA molecule to another predetermined RNA molecule.

In another embodiment, the invention features a nucleic acid sensor molecule comprising an enzymatic nucleic acid component and one or more sensor components, wherein, in response to an interaction of a single stranded DNA (ssDNA) with the nucleic acid sensor molecule in a system, the enzymatic nucleic acid component catalyzes a chemical reaction resulting in ligation of a predetermined RNA molecule to another predetermined RNA molecule.

In yet another embodiment, the invention features a nucleic acid sensor molecule comprising an enzymatic nucleic acid component and one or more sensor components, wherein, in response to an interaction of a peptide with the nucleic acid sensor molecule in a system, the enzymatic nucleic acid component catalyzes a chemical reaction resulting in ligation of a predetermined RNA molecule to another predetermined RNA molecule.

In still another embodiment, the invention features a nucleic acid sensor molecule comprising an enzymatic nucleic acid component and one or more sensor components, wherein, in response to an interaction of a protein with the nucleic acid sensor molecule in a system, the enzymatic nucleic acid component catalyzes a chemical reaction resulting in ligation of a predetermined RNA molecule to another predetermined RNA molecule.

In one embodiment, the invention features a method comprising: (a) contacting a nucleic acid sensor molecule of the invention with a system comprising at least one ssRNA having a SNP under conditions suitable for the enzymatic nucleic acid component of the nucleic acid sensor molecule to catalyze a chemical reaction resulting in a detectable response; and (b) assaying for the detectable response.

In another embodiment, the invention features a method comprising: (a) contacting a nucleic acid sensor molecule of the invention with a system comprising at least one ssDNA having a SNP under conditions suitable for the enzymatic nucleic acid component of the nucleic acid sensor molecule to catalyze a chemical reaction resulting in a detectable response; and (b) assaying for the detectable response.

In another embodiment, the invention features a method comprising: (a) contacting a nucleic acid sensor molecule of the invention with a system comprising at least one peptide under conditions suitable for the enzymatic nucleic acid component of the nucleic acid sensor molecule to catalyze a chemical reaction resulting in a detectable response; and (b) assaying for the detectable response.

In yet another embodiment, the invention features a method comprising: (a) contacting a nucleic acid sensor molecule of the invention with a system comprising at least one protein, under conditions suitable for the enzymatic nucleic acid component of the nucleic acid sensor molecule to catalyze a chemical reaction resulting in a detectable response; and (b) assaying for the detectable response.

In one embodiment, the invention features a method comprising contacting a nucleic acid sensor molecule of the invention with a system comprising at least one ssRNA under conditions suitable for the enzymatic nucleic acid component of the nucleic acid sensor molecule to cleave a predetermined RNA molecule.

In another embodiment, the invention features a method comprising the steps of contacting a nucleic acid sensor molecule of the invention with a system comprising at least one ssDNA under conditions suitable for the enzymatic nucleic acid component of the nucleic acid sensor molecule to cleave a predetermined RNA molecule

In yet another embodiment, the invention features a method comprising the steps of contacting a nucleic acid sensor molecule of the invention with a system comprising at least one peptide under conditions suitable for the enzymatic nucleic acid component of the nucleic acid sensor molecule to cleave a predetermined RNA molecule.

In another embodiment, the invention features a method comprising the steps of contacting a nucleic acid sensor molecule of the invention with a system comprising at least one protein, under conditions suitable for the enzymatic nucleic acid component of the nucleic acid sensor molecule to cleave a predetermined RNA molecule.

In one embodiment, the invention features a method comprising contacting a nucleic acid sensor molecule of the invention with a system comprising at least one ssRNA having a SNP under conditions suitable for the enzymatic nucleic acid component of the nucleic acid sensor molecule to ligate a predetermined RNA molecule to another predetermined RNA molecule.

In another embodiment, the invention features a method comprising the steps of contacting a nucleic acid sensor molecule of the invention with a system comprising at least one ssDNA having a SNP under conditions suitable for the enzymatic nucleic acid component of the nucleic acid sensor molecule to ligate a predetermined RNA molecule to another predetermined RNA molecule.

In yet another embodiment, the invention features a method comprising the steps of contacting a nucleic acid sensor molecule of the invention with a system comprising at least one peptide under conditions suitable for the enzymatic nucleic acid component of the nucleic acid sensor molecule to ligate a predetermined RNA molecule to another predetermined RNA molecule.

In another embodiment, the invention features a method comprising the steps of contacting a nucleic acid sensor molecule of the invention with a system comprising at least one protein, under conditions suitable for the enzymatic nucleic acid component of the nucleic acid sensor molecule to ligate a predetermined RNA molecule to another predetermined RNA molecule.

In one embodiment, the invention features a method of using the nucleic acid sensor molecules of the invention to determine the function or validate a predetermined gene target, a predetermined RNA target, a predetermined peptide target, or a predetermined protein target.

In another embodiment, the invention features a method of using the nucleic acid sensor molecules of the invention to determine a genotype or to characterize single nucleotide polymorphisms SNPs in a gene or genome. In another embodiment, the invention features a method of using the nucleic acid sensor molecules of the invention to determine SNP scoring.

In another embodiment, the invention features a method of using the nucleic acid sensor molecules of the invention to determine a proteome, for example a disease specific proteome or treatment specific proteome. In yet another embodiment, the invention features a method of using the nucleic acid sensor molecules of the invention to determine a proteome map or to determine proteome scoring.

In one embodiment, the invention features a method of using the nucleic acid sensor molecules of the invention to determine the dosage of a therapy used in treating a patient, to determine susceptibility of a patient to disease, to determine drug metabolism in a patient, to select a patient for a clinical trail, to determine a choice of therapy in a patient, or to treat a patient.

In another embodiment, the detection of a chemical reaction in a method of the invention is indicative of the presence of the target signaling agent in the system.

In another embodiment, the absence of a chemical reaction in a method of the invention is indicative of the system lacking the target signaling agent.

In one embodiment, a system of the invention is an in vitro system, for example a sample derived from the group consisting of a patient, plant, water, beverage, food preparation, and soil.

In another embodiment, a system of the invention is an in vivo system, for example a bacteria, a bacterial cell, a fungus, a fungal cell, a virus, a plant, plant cell, mammal, mammalian cell, human, or human cell.

In another embodiment, a component of a nucleic acid sensor molecule of the invention comprises a hammerhead, hairpin, inozyme, G-cleaver, Zinzyme, RNase P EGS nucleic acid, DNAzyme or Amberzyme motif.

In one embodiment, a chemical reaction of a nucleic sensor molecule of the invention comprises cleavage of a phosphodiester internucleotide linkage, ligation of a predetermined nucleic acid molecule to the nucleic acid sensor molecule, ligation of a predetermined nucleic acid molecule to another predetermined nucleic acid molecule, isomerization, phosphorylation of a peptide or protein, dephosphorylation of a peptide or protein, RNA polymerase activity, an increase or decrease in fluorescence, an increase or decrease in enzymatic activity, an increase or decrease in the production of a precipitate, an increase or decrease in chemoluminescence, or an increase or decrease in radioactive emission.

In another embodiment, the invention features a kit comprising a nucleic acid sensor molecule of the invention.

In another embodiment, the invention features an array of nucleic acid sensor molecules comprising a predetermined number of nucleic acid sensor molecules of the invention. In one embodiment, a nucleic acid sensor molecule of the instant invention is attached to a solid surface. Preferably, the surface of the instant invention comprises silicon-based chips, silicon-based beads, controlled pore glass, polystyrene, cross-linked polystyrene, nitrocellulose, biotin, plastics, metals and polyethylene films.

In another embodiment, the invention features a nucleic acid sensor molecule comprising an enzymatic nucleic acid component and one or more sensor components, wherein, in response to an interaction of a HCV peptide with the nucleic acid sensor molecule in a system, the enzymatic nucleic acid component catalyzes a chemical reaction resulting in resulting in the cleavage of a predetermined RNA molecule associated with a disease, for example HCV RNA.

In yet another embodiment, the invention features a nucleic acid sensor molecule comprising an enzymatic nucleic acid component and one or more sensor components, wherein, in response to an interaction of a HCV protein, for example a HCV core protein or coat protein, with the nucleic acid sensor molecule in a system, the enzymatic nucleic acid component catalyzes a chemical reaction resulting in resulting in the cleavage of a predetermined RNA molecule associated with a disease, for example HCV RNA.

In one embodiment, a nucleic acid sensor molecule of the invention comprises a sensor component having a sequence derived from the HCV 5′-UTR, for example structural domains IIIa-IIIf, I, II or IV.

In another embodiment, the invention features a pharmaceutical composition comprising a nucleic acid sensor molecule in a pharmaceutically acceptable carrier.

In one embodiment, the invention features a method of administering to a cell, for example a mammalian cell or human cell, a nucleic acid sensor molecule of the invention comprising contacting the cell with the nucleic acid sensor molecule under conditions suitable for the administration. The method of administration can be in the presence of a delivery reagent, for example a lipid, cationic lipid, phospholipid, or liposome.

In another embodiment, the invention features a cell, for example a human cell, plant cell, bacterial cell, or fungal cell, including a nucleic acid sensor molecule of the invention.

In another embodiment, the invention features an expression vector comprising a nucleic acid sequence encoding at least one nucleic acid sensor molecule of the invention in a manner which allows expression of the nucleic acid sensor molecule.

In yet another embodiment, the invention features a mammalian cell, for example a human cell, including an expression vector of the invention.

In one embodiment, an expression vector of the invention further comprises a sequence for a nucleic acid sensor molecule complementary to an RNA having HCV sequence.

In another embodiment, an expression vector of the invention comprises a nucleic acid sequence encoding two or more nucleic acid sensor molecules, which may be the same or different.

In another embodiment, a peptide contemplated by the invention is a viral peptide, for example a peptide derived from HCV, HBV, HIV, HPV, HTLV-1, CMV, HSV, RSV, Rhinovirus, WNV, Hantavirus, Ebola virus, or Encephalovirus.

In another embodiment, a protein contemplated by the invention is a viral protein, for example a protein derived from HCV, HBV, HIV, HPV, HTLV-1, CMV, HSV, RSV, Rhinovirus, WNV, Hantavirus, Ebola virus, or Encephalovirus.

In another embodiment, a predetermined RNA of the invention is associated with HCV infection.

In another embodiment, the method of the instant invention is carried out more than once.

### DETAILED DESCRIPTION OF THE INVENTION

The present invention features compositions and methods for the detection and/or amplification of specific target signaling agents and target signaling molecules in a system using nucleic acid sensor molecules.

In one embodiment, the present invention features a nucleic acid sensor molecule comprising an enzymatic nucleic acid component and one or more sensor components that, in response to an interaction of a target signaling agent, the enzymatic nucleic acid component catalyzes a chemical reaction in which the activity or physical properties of a reporter molecule is modulated. Preferably, the chemical reaction in which the activity or physical properties of a reporter molecule is modulated results in a detectable response.

In a preferred embodiment, the present invention features a nucleic acid sensor molecule comprising an enzymatic nucleic acid component and one or more sensor components, wherein, in response to an interaction of a target signaling agent or target signaling molecule with the nucleic acid sensor molecule, the enzymatic nucleic acid component catalyses a chemical reaction involving covalent attachment of at least a portion of a reporter molecule.

Preferably, the chemical reaction in which a reporter molecule is covalently attached to the nucleic acid sensor molecule is selected from the group consisting of ligation, transesterification, phosphorylation, carbon-carbon bond formation, amide bond formation, peptide bond formation, and disulfide bond formation.

In another embodiment, the present invention features a nucleic acid sensor molecule comprising an enzymatic nucleic acid component and one or more sensor components, wherein, in response to an interaction of a target signaling molecule with the nucleic acid sensor molecule, the enzymatic nucleic acid component can carry out a chemical reaction that modulates the activity or properties of the reporter molecule. Preferably, the chemical reaction in which the activity of a reporter molecule is modulated is selected from the group consisting of phosphorylation, dephosphorylation, isomerization, polymerization, amplification, helicase activity, transesterification, ligation, hydration, hydrolysis, alkylation, dealkylation, halogenation, dehalogenation, esterification, desterification, hydrogenation, dehydrogenation, saponification, desaponification, amination, deamination, acylation, deacylation, glycosylation, deglycosylation, silation, desilation, hydroboration, epoxidation, peroxidation, carboxylation, decarboxylation, substitution, elimination, oxidation, and reduction or a combination thereof.

In one embodiment, the invention features a nucleic acid sensor molecule comprising an enzymatic nucleic acid component and one or more sensor components, wherein, in response to an interaction of a target signaling molecule with the nucleic acid sensor molecule, the enzymatic nucleic acid component can carry out a chemical reaction involving isomerization of at least a portion of a reporter molecule.

In another embodiment, the invention features a nucleic acid sensor molecule comprising an enzymatic nucleic acid component and one or more sensor components, wherein, in response to an interaction of a target signaling molecule with the nucleic acid sensor molecule, the enzymatic component catalyses a chemical reaction on a non-oligonucleotide-based portion of a reporter molecule selected from the group consisting of phosphorylation and dephosphorylation.

In another embodiment, the reporter molecule of the instant invention is selected from the group consisting of molecular beacons, small molecules, fluorophores, chemophores, ionophores, radio-isotopes, photophores, peptides, proteins, enzymes, antibodies, nucleic acids, and enzymatic nucleic acids or a combination thereof (see for example in Singh et al., 2000, Biotech., 29, 344; Lizardi et al., U.S. Pat. Nos. 5,652,107 and 5,118,801).

Using such reporter molecules and others known in the art, the detectable response of the instant invention can be monitored by, for example, a change in fluorescence, color change, UV absorbance, phosphorescence, pH, optical rotation, isomerization, polymerization, temperature, mass, capacitance, resistance, and emission of radiation.

Detection of the target signaling event via the chemical reaction or the change in activity or physical properties of the reporter molecule can be assayed by methods known in the art. Amplification of the target signaling event via the chemical reaction or the change in activity or physical properties of the reporter molecule is accomplished by methods known in the art, for example, modulating polymerase activity. Modulation of polymerase activity can increase polymerization in a chemical reaction, for example, a polymerase chain reaction (PCR) system, resulting in amplification of a target signaling molecule or reporter molecule.

In one embodiment, a linker region (see, for example L in FIGS. 17 a,b,c) can join the nucleic acid sensor molecule to a reporter molecule, for example, via ligation activity of an enzymatic nucleic acid component of the nucleic acid sensor molecule in response to a target signaling agent's interaction with a sensor component of the nucleic acid sensor molecule.

In another embodiment, the invention features a nucleic acid sensor molecule, wherein said reporter molecule comprises the formula:

R₁—L—R₂

wherein R1 is selected from the group consisting of alkyl, alkoxy, hydrogen, hydroxy, sulfhydryl, ester, anhydride, acid halide, amide, nitrile, phosphate, phosphonate, nucleoside, nucleotide, oligonucleotide; R2 is selected from the group consisting of molecular beacons, small molecules, fluorophores, chemophores, ionophores, radio-isotopes, photophores, peptides, proteins, enzymes, antibodies, nucleic acids, and enzymatic nucleic acids; L represents a linker which can be present or absent, and “—” represents a chemical bond

In another embodiment, the invention features a nucleic acid sensor molecule of claim 3, wherein said reporter molecule comprises the formula:

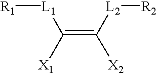

wherein R1 and R2 each represent compounds, which can be the same or different, that generate a detectable signal or quench a detectable signal when an isomerization reaction is catalyzed, selected from the group consisting of molecular beacons, small molecules, fluorophores, chemophores, ionophores, radio-isotopes, photophores, peptides, proteins, enzymes, antibodies, nucleic acids, and enzymatic nucleic acids; L1 and L2 each represent a linker which can be the same or different and which can be present or absent; X1 and X2 each represent an atom, compound, or molecule that can be the same or different, and “—” represents a chemical bond. In another preferred embodiment, the invention features a nucleic acid sensor molecule of claim 3, wherein said reporter molecule comprises the formula:

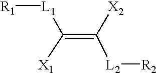

wherein R1 and R2 each represent compounds, which can be the same or different, that generate a detectable signal or quench a detectable signal when an isomerization reaction is catalyzed, selected from the group consisting of molecular beacons, small molecules, fluorophores, chemophores, ionophores, radio-isotopes, photophores, peptides, proteins, enzymes, antibodies, nucleic acids, and enzymatic nucleic acids; L1 and L2 each represent a linker which can be the same or different and which can be present or absent; X1 and X2 represent an atom, compound, or molecule that can be the same or different, and “—” represents a chemical bond.

In another embodiment, the reaction catalyzed by the enzymatic nucleic acid component of the nucleic acid sensor or nucleic acid sensor molecule with the reporter molecule of the invention features catalytic activity, for example, cleavage activity, ligation activity, isomerization activity, phosphorylation activity, dephosphorylation activity, amplification activity, and/or polymerase activity.

The invention also features a method comprising the steps of: (a) contacting a nucleic acid sensor molecule comprising an enzymatic nucleic acid component and one or more sensor components, and a reporter molecule with a system under conditions suitable for the enzymatic nucleic acid component of the nucleic acid sensor molecule to attach at least a portion of the reporter molecule to the nucleic acid sensor molecule in the presence of a target signaling agent; and (b) assaying for the attachment of the reporter molecule to the nucleic acid sensor molecule.

In another embodiment, the invention features a method comprising the steps of: (a) contacting a nucleic acid sensor molecule comprising an enzymatic nucleic acid component and one or more sensor components, and a reporter molecule with a system under conditions suitable for the enzymatic nucleic acid component of the nucleic acid sensor molecule to isomerize at least a portion of the reporter molecule in the presence of a target signaling agent; and (b) assaying for the isomerization reaction.

In yet another embodiment, the invention features a method comprising the steps of: (a) contacting a nucleic acid sensor molecule comprising an enzymatic nucleic acid component and one or more sensor components, and a reporter molecule with a system under conditions suitable for the enzymatic nucleic acid component of the nucleic acid sensor molecule to phosphorylate a non-oligonucleotide-based portion of the reporter molecule in the presence of a target signaling agent; and (b) assaying for the phosphorylation reaction.

In still another embodiment, the invention features a method comprising the steps of: (a) contacting a nucleic acid sensor molecule comprising an enzymatic nucleic acid component and one or more sensor components, and a reporter molecule with a system under conditions suitable for the enzymatic nucleic acid component of the nucleic acid sensor molecule to dephosphorylate a non-oligonucleotide-based portion of the reporter molecule in the presence of a target signaling agent; and (b) assaying for the dephosphorylation reaction.

In any of the above-described inventive methods, the system can be an in vitro system. Preferably, the in vitro system is a sample derived from the group consisting of a patient, plant, water, beverage, food preparation, and soil. In any of the above-described inventive methods, the enzymatic nucleic acid component of said nucleic acid sensor molecule can be a hammerhead, hairpin, inozyme, G-cleaver, Zinzyme, RNase P EGS nucleic acid and Amberzyme motif. Also, in any of the above-described inventive methods, the enzymatic nucleic acid component of said nucleic acid sensor molecule can be a DNAzyme.

In any of the above-described methods, the detection of a chemical reaction can be indicative of the presence of the target signaling molecule in the system. In any of the above-described methods, the absence of a chemical reaction is indicative of the system lacking the target signaling molecule.

In one embodiment, the reporter molecule of the instant invention is selected from the group consisting of molecular beacons, small molecules, fluorophores, chemophores, ionophores, radio-isotopes, photophores, peptides, proteins, enzymes, antibodies, nucleic acids, and enzymatic nucleic acids or a combination thereof (see for example in Singh et al., 2000, Biotech., 29, 344; Lizardi et al., U.S. Pat. Nos. 5,652,107 and 5,118,801).

Using such reporter molecules and others known in the art, the detectable response of the instant invention can be monitored by, for example, a change in fluorescence, color change, UV absorbance, phosphorescence, pH, optical rotation, isomerization, polymerization, temperature, mass, capacitance, resistance, and emission of radiation.

Detection of the target signaling event via the chemical reaction or the change in activity or physical properties of the reporter molecule can be assayed by methods known in the art. Amplification of the target signaling event via the chemical reaction or the change in activity or physical properties of the reporter molecule is accomplished by methods known in the art, for example, modulating polymerase activity. Modulation of polymerase activity can increase polymerization in a chemical reaction, for example, a polymerase chain reaction (PCR) system, resulting in amplification of a target signaling molecule or reporter molecule.

The present invention features a nucleic acid-based sensor molecule comprising an enzymatic nucleic acid component and one or more sensor components. The nucleic acid sensor molecule is selected for having catalytic activity only through interaction with a target signaling agent such that in response to an interaction of the target signaling agent with at least one sensor component, the enzymatic portion of the nucleic acid sensor molecule catalyzes a chemical reaction.

In one embodiment, the nucleic acid sensor molecule comprises an enzymatic nucleic acid component and one or more sensor components, wherein the enzymatic nucleic acid component and sensor component(s) are distinct moieties. In one embodiment, the nucleic acid sensor molecule comprises an enzymatic nucleic acid component and one or more sensor components, wherein the distinct enzymatic nucleic acid component and sensor component(s) are joined by a linker region. Thus, in one embodiment, a linker region joins one or more enzymatic nucleic acid components to one or more sensor components in the nucleic acid sensor molecules of the instant invention.

As discussed above, the chemical reaction carried out by the nucleic acid sensor molecule can comprise a reaction in which a reporter molecule or a portion of a reporter molecule becomes covalently attached to the nucleic acid sensor molecule. Thus, in another embodiment, the nucleic acid sensor molecule comprises an enzymatic nucleic acid component and one or more sensor components, wherein the distinct enzymatic nucleic acid component and sensor component(s) are joined by a covalent bond. In one embodiment, the chemical reaction carried out by the nucleic acid sensor molecule comprises a reaction in which a reporter molecule becomes covalently attached to the nucleic acid sensor molecule that is immobilized on a solid support or surface. Suitable solid surfaces include silicon-based chips, silicon-based beads, controlled pore glass, polystyrene, and cross-linked polystyrene nitrocellulose, biotin, plastics, metals and polyethylene films. In another embodiment, the nucleic acid sensor molecule comprises an enzymatic nucleic acid component and one or more sensor components, wherein a sensor component of a nucleic acid sensor molecule of the instant invention is an integral part of the enzymatic nucleic acid component of the nucleic acid sensor molecule. Specifically, in preferred embodiments, one or more sensor components of a nucleic acid sensor molecule shares sequence with the enzymatic nucleic acid component of the nucleic acid sensor molecule and is necessary for the activity of the enzymatic nucleic acid component. The sensor component can also be part of the enzymatic nucleic acid component of the nucleic acid sensor molecule.

In the presence of a target signaling molecule, the sensor component activates or facilitates a chemical reaction. Alternatively, in the presence of a target signaling molecule, the sensor component inhibits a chemical reaction from taking place.

In other embodiments, the invention features the use of at least one reporter molecule, at least one target signaling molecule, and a nucleic acid sensor molecule which is comprised of an enzymatic nucleic acid component joined by a linker to one or more sensor components, where a sensor component, for example, is complementary to one or more sequences within the enzymatic nucleic acid component. The enzymatic nucleic acid component's ability, in the nucleic acid sensor or nucleic acid sensor molecule, to catalyze a reaction is inhibited by the interaction of one or more sensor components. However, in the presence of one or more distinct target signaling molecules, the sensor component interacts with its respective target signaling molecule preferentially, allowing the nucleic acid sensor molecule to interact with a reporter molecule to catalyze a reaction. A catalytic reaction then takes place on the reporter molecule, for example, cleavage or ligation of the reporter molecule, the rate of which can then be measured by standard assays described herein and otherwise well known in the art.

In another embodiment, the invention features a method for the detection and/or amplification of specific target signaling molecules in a system using at least one reporter molecule, at least one target signaling molecule, and a nucleic acid sensor molecule which comprises an enzymatic nucleic acid component and at least one separate sensor component, where the sensor component or components interacts with one or more sequences within the nucleic acid sensor molecule. The enzymatic nucleic acid component's ability, in the nucleic acid sensor molecule, to catalyze a reaction is inhibited by the interaction of at least one sensor component. However, in the presence of a target signaling molecule, the sensor component preferentially interacts with the enzymatic nucleic acid component, which allows the nucleic acid sensor molecule to interact with a reporter molecule and become functional. A catalytic reaction then takes place on the reporter molecule, for example, cleavage or ligation of the reporter molecule, the rate of which can then be measured by standard assays described herein and otherwise well known in the art.

In one embodiment, the invention features a method for the detection and/or amplification of a specific target signaling molecule in a system using at least one reporter molecule, at least one target signaling molecule, and a nucleic acid sensor molecule which comprises an enzymatic nucleic acid component. The nucleic acid sensor molecule is selected for having catalytic activity only through interaction with the target signaling molecule. In the absence of the target signaling molecule, the nucleic acid sensor molecule is inactive. In the presence of a target signaling molecule the nucleic acid sensor molecule can adopt an active conformation and become functional. A catalytic reaction then takes place on the reporter molecule, for example, cleavage or ligation of the reporter molecule, the rate of which can then be measured by standard assays well known in the art. Alternatively, the nucleic acid sensor molecule can be selected to be inhibited through interaction with the target signaling molecule, such that interaction with the target causes the nucleic acid sensor molecule to adopt an inactive conformation and become non-active.

Thus in one embodiment, the present invention features a method comprising the steps of: (a) contacting a nucleic acid sensor molecule which comprises: (i) an enzymatic nucleic acid component comprising a substrate binding region and a catalytic region; and (ii) a sensor component comprising a nucleic acid sequence that upon interacting with a complementary sequence in the enzymatic nucleic acid component inhibits the activity of the enzymatic nucleic acid component, and a reporter molecule comprising a nucleic acid sequence complementary to the substrate binding region of the enzymatic nucleic acid component of the nucleic acid sensor molecule with a system under conditions suitable for the enzymatic nucleic acid component of the nucleic acid sensor molecule to catalyze cleavage of the reporter molecule in the presence of a target signaling molecule; and (b) assaying for the cleavage reaction of step (a).

In one embodiment of the inventive method, the cleavage of the reporter molecule is indicative of the presence of the target signaling molecule in the system. The absence of cleavage of the reporter molecule is indicative of the system lacking the target signaling molecule.

In another embodiment, the present invention features a method comprising the steps of: (a) contacting a nucleic acid sensor molecule which comprises: (i) an enzymatic nucleic acid component comprising a substrate binding region and a catalytic region; and (ii) a sensor component comprising a nucleic acid sequence that upon interacting with a complementary sequence in the enzymatic nucleic acid component inhibits the activity of the enzymatic nucleic acid component, and a reporter molecule comprising a nucleic acid sequence complementary to the substrate binding region of the enzymatic nucleic acid component of the nucleic acid sensor molecule with a system under conditions suitable for the enzymatic nucleic acid component of the nucleic acid sensor molecule to catalyze a ligation reaction involving the reporter molecule in the presence of a target signaling molecule; and (b) assaying for the ligation reaction in step (a).

In one embodiment of the inventive method, the ligation reaction causes at least a portion of the reporter molecule to be attached to the nucleic acid sensor molecule. In another embodiment, the ligation reaction causes at least a portion of the reporter molecule to be attached to a separate molecule. Also, in one embodiment of the inventive method, the ligation of the reporter molecule is indicative of the presence of the target signaling molecule in the system. The absence of ligation of the reporter molecule is indicative of the system lacking the target signaling molecule.

In any of the above-described inventive methods, the system can be an in vitro system. Preferably, the in vitro system is a sample derived from the group consisting of a patient, plant, water, beverage, food preparation, and soil.

In any of the above described methods, the target signaling molecule can be an RNA, DNA, analog of RNA or analog of DNA. Thus, for example, the reporter molecule can be an RNA, DNA, RNA analog, or DNA analog. Also, in any of the described methods, wherein the targeting signaling molecule is an RNA, preferably the RNA is derived from a bacteria (e.g. Corynebacteria, Pneumococci, Streptococci, Staphylococci, enteric bacilli, mycobacteria, spirochetes, chlamydiae), virus (e.g. Hepatitis C virus (HCV), Hepatitis B virus (HBV), human immunodeficiency virus (HIV), human papilloma virus (HPV), poliovirus, West Nile virus (WNV), Human T-cell Lyphotrophic Virus Type 1 (HTLV-1), cytomegalovirus (CMV), Herpes Simplex Virus (HSV), respiratory syncytial virus (RSV),. influenza virus, rhinovirus, foot and mouth disease virus, ebola virus, dengue fever virus, feline leukemia virus (FLV)), fungi (e.g. genera Aspergillus Penicillium and Cladosporium), plant (e.g. corn, soy, cotton, wheat) or mammalian (e.g. human, mouse, rat, cat, dog, monkey) genome. In one embodiment, the invention features a method of detecting and/or amplifying target signaling molecules, wherein said target signaling molecule is a nucleic acid sequence such as RNA and/or DNA, in a system, preferably a mammalian system, comprising the steps of (1) contacting the system with the nucleic acid sensor molecule and the reporter molecule under conditions suitable for the target signaling molecule, if present in the sample, to interact with the sensor component of the nucleic acid sensor molecule, such that the enzymatic nucleic acid component of the sensor molecule can interact with the reporter molecule to catalyze a reaction; and (2) measuring of the extent of the reaction catalyzed by the enzymatic nucleic acid component of the sensor molecule, indicating the presence of the target signaling molecule. If the target signaling molecule is not present in the sample, then no reaction above the background will be detected. The reporter molecule can be contacted with the system after the system is allowed to interact with the nucleic acid sensor molecule.

In another embodiment, the invention features a method of detecting and/or amplifying a target signaling molecule, wherein the target signaling molecule is RNA sequence derived from a virus (e.g. Hepatitis C virus (HCV), Hepatitis B virus (HBV), human immunodeficiency virus (HIV), human papilloma virus (HPV), poliovirus, West Nile virus (WNV), cytomegalovirus (CMV), Herpes Simplex Virus (HSV), respiratory syncytial virus (RSV), influenza virus, rhinovirus, foot and mouth disease virus, ebola virus, dengue fever virus, feline leukemia virus (FLV)), fungi (e.g. genera Aspergillus Penicillium and Cladosporium), plant (e.g. corn, soy, cotton, wheat) or mammalian (e.g. human, mouse, rat, cat, dog, monkey) genome, bacteria (e.g. Corynebacteria, Pneumococci, Streptococci, Staphylococci, enteric bacilli, mycobacteria, spirochetes, chlamydiae), mycoplasma or other infectious disease agent, in a system, where the system is a biological sample from a patient, animal, blood, food material, water, and/or other potential sources for infectious disease agents. The method comprises the steps of (1) contacting the system with the nucleic acid sensor molecule, where the nucleic acid sensor molecule comprises an sensor component and an enzymatic nucleic acid component, under conditions suitable for preferential interaction of the sensor component with the target signaling molecule that can be present in the system; (2) contacting the system with a reporter molecule under conditions suitable for the enzymatic nucleic acid component of the nucleic acid sensor molecule to catalyze a reaction with the reporter molecule; and (3) detecting the target signaling molecule by measuring any reaction catalyzed in step (2).

In another embodiment, the invention features a method of the detecting and/or amplifying a target signaling molecule, wherein the target signaling molecule is RNA sequence derived from a virus (e.g. Hepatitis C virus (HCV), Hepatitis B virus (HBV), human immunodeficiency virus (HIV), human papilloma virus (HPV), poliovirus, West Nile virus (WNV), cytomegalovirus (CMV), Herpes Simplex Virus (HSV), respiratory syncytial virus (RSV), influenza virus, rhinovirus, foot and mouth disease virus, ebola virus, dengue fever virus, feline leukemia virus (FLV)), fungi (e.g. genera Aspergillus Penicillium and Cladosporium), plant (e.g. corn, soy, cotton, wheat) or mammalian (e.g. human, mouse, rat, cat, dog, monkey) genome, bacteria (e.g. Corynebacteria, Pneumococci, Streptococci, Staphylococci, enteric bacilli, mycobacteria, spirochetes, chlamydiae), mycoplasma or other infectious disease agent, or other potential sources for infectious disease agents. The method comprises the steps of (1) contacting the reporter molecule with a mixture, comprising the system and the nucleic acid sensor molecule, under conditions suitable for the active configuration of the enzymatic nucleic acid component of the nucleic acid sensor molecule to interact with the reporter molecule to catalyze a reaction; and (2) detecting the target signaling molecule by measuring the reaction catalyzed in step (1). If the target signaling molecule is not present in the system, then the enzymatic nucleic acid component will not catalyze a reaction with the reporter molecule and there will not be a signal to measure.

In another embodiment, one or more nucleic acid sensor molecules are attached to a solid support, for example, a silicon-based surface. Each nucleic acid sensor molecule can be attached via one of its termini by a spacer molecule to allow the nucleic acid sensor molecule to adopt the appropriate conformations without hindrance from the underlying solid support. A test mixture is contacted with one or more nucleic acid sensor molecules, and the mixture is contacted with the solid support. Measurement of a signal generated by the nucleic acid sensor molecule in response to interaction with a target signaling molecule at each address of the array reveals the concentration of each target signaling molecule in the test mixture.

In any of the above methods, the enzymatic nucleic acid component of said nucleic acid sensor molecule can be a hammerhead, hairpin, inozyme, G-cleaver, Zinzyme, RNase P EGS nucleic acid and Amberzyme motif.

In any of the above methods, the enzymatic nucleic acid component of said nucleic acid sensor molecule can be a DNAzyme.

In any of the above methods, the reporter molecule can comprise a detectable label selected from the group consisting of chromogenic substrate, fluorescent labels, chemiluminescent labels, and radioactive labels.

In any of the above methods, the reporter molecule can be immobilized on a solid support, preferably comprising silicon-based chips, silicon-based beads, controlled pore glass, polystyrene, cross-linked polystyrene, nitrocellulose, biotin, plastics, metals and polyethylene films.

In one embodiment of the inventive method, the sensor component of the nucleic acid sensor molecule is RNA, DNA, analog of RNA or analog of DNA.

In another embodiment, the sensor component of the nucleic acid sensor molecule is covalently linked to the nucleic acid sensor molecule by a linker. Suitable linkers include, for example, one or more nucleotides, abasic moiety, polyether, polyamine, polyamide, peptide, carbohydrate, lipid, and polyhydrocarbon compounds, and any combination thereof.

In another embodiment, the sensor component of the nucleic acid sensor molecule is not covalently linked to the nucleic acid sensor molecule.

In one embodiment, the nucleic acid sensor molecules of the invention are used to detect target signaling agents involved in human and animal disease, for example viruses, bacteria, proteins, other pathogens and toxins. Examples of viral target signaling agents include but are not limited to Hepatitis C virus (HCV), Hepatitis B virus (HBV), human immunodeficiency virus (HIV), human papilloma virus (HPV), poliovirus, West Nile virus (WNV), cytomegalovirus (CMV), Herpes Simplex Virus (HSV), respiratory syncytial virus (RSV), influenza virus, rhinovirus, foot and mouth disease virus, ebola virus, dengue fever virus, feline leukemia virus (FLV), and others. Examples of bacterial target signaling agents include but are not limited to Corynebacteria, Pneumococci, Streptococci, Staphylococci, enteric bacilli, mycobacteria, spirochetes, chlamydiae, and others. Examples of protein target signaling agents include but are not limited to prions, for example CVJ and BSE associated prions, signal transduction proteins, tyrosine kinases, phosphatases, phosphorylases, dephosphorylases, polymerases and others. Examples of other parasite target signaling agents include but are not limited to pathogenic agents related to malaria, lyme disease (Borrelia burgdorferi), sleeping sickness, giardia, and cryptsporidia. Examples of toxin target signaling agents include but are not limited to lead, mercury, asbestos, pesticides, herbicides, PCBs, and other organic and inorganic compounds.

The present invention also provides kits for the detection of particular targets in test mixtures. The kit comprises separate components containing solutions of a nucleic acid sensor molecule specific for a particular target signaling agent, and containing solutions of the appropriate reporter molecules. In some embodiments, the kit comprises a solid support to which is attached the nucleic acid sensor molecule to the particular target. In further embodiments, the kit further comprises a component containing a standardized solution of the target. With this solution, it is possible for the user of the kit to prepare a graph or table of the detectable signal (for example, fluorescence units vs. target concentration); this table or graph is then used to determine the concentration of the target in the test mixture. Devices that automate the manipulation of such kits, perform the repeated function of the kits, combine various steps of kits, or that generate data from the kits are further contemplated by the instant invention.

In one embodiment, the invention features the use of nucleic acid sensor molecules in nucleic acid based electronics, including nucleic acid-based switches, semiconductor circuits, and computers. The present invention also provides for the detection of signaling agents by means of nucleic acid circuit arrays, including the use of nucleic acid sensor molecules in nucleic acid based switches, semiconductor circuits, and computers. Recent research has indicated the capacity for nucleic acids, specifically DNA, to act as a conductor, semiconductor, or insulator based on nucleotide sequence (Porath et al., 2000, Nature, 403, 635-638; Kelley and Barton, 1999, Science, 283, 375-381; and Jortner et al., 1998, PNAS USA., 95, 12759-12765; and Geise et al., 1999, Angew. Chem, Int. Ed. Engl., 38, 996). The use of nucleic acid sensor molecules in cis or in trans in nucleic acid circuits is further contemplated by the instant invention. Such use can provide a means for creating complex nucleic acid based circuits, enabling the development of nano-computing devices, biosensors, and biologically integrated circuits for in vitro and in vivo use through the ability for the nucleic acid sensor molecule to either cleave or ligate a nucleic acid in cis or in trans in response to signaling agents.

In another embodiment, the nucleic acid sensor molecules are used in DNA computing applications. For example, the use of unique sequences of nucleic acid can be used to solve complex problems (Guarnieri et al., 1996, Science, 278, 361; Ouyang et al., 1997, Science, 278, 446). Structural motifs of nucleic acid are emerging as unique tools in solving problems in nucleic acid computing (Sakamoto et al., 2000, Science, 288, 1152). The use of nucleic acid sensor molecules that recognize specific sequence and structural information and that modulate this information are of value to nucleic acid computing.

In another embodiment, the invention features the use of nucleic acid sensor molecules in nucleic acid-based electronics utilized in nucleic acid computing applications. The combined use of nucleic acid sensor molecules in nucleic acid-based electronics and in nucleic acid computing bridges an important gap in the ability to generate signal output from nucleic acid-based computations. For example, the detection of specific nucleic acid sequences or structures that represent the solution to a problem or convey other information in a nucleic acid-based computation system by nucleic acid sensor molecules can result in the detection of a signal generated by a reporter molecule.

In one embodiment, the invention specifically features a process whereby a nucleic acid signaling molecule is used in a nucleic acid circuit. In response to a target signaling agent, for example current, the nucleic acid sensor molecule catalyzes a chemical reaction comprising ligation in response to a predetermined current or cleavage in response to a predetermined current. The nucleic acid circuit is thereby modulated between an open and a closed state based on the predetermined input current that is applied to the circuit. A plurality of such circuits that comprise nucleic acid sensor modulation can be used in a variety of electronic devices, and can substitute solid state or silicon-based circuits in such devices. For example, computer processors comprising a plurality of nucleic acid sensor molecule based-circuits can be used in a computer device. Open and closed nucleic acid sensor molecule based-circuits can be used to generate or respond to binary code. Processing of nucleic acids by nucleic acid sensor molecules can be used to generate more complex code, for example where particular nucleic acid sequences represent different code variables.

Electronic devices comprising nucleic acid circuits which comprise nucleic acid sensor molecules of the invention are advantageous over the current state of the art circuits in terms of the absolute minimum size of the circuit and the device and the degree of modulation enabled by nucleic acid sensor molecules. For example, since nucleic acid sensor molecule-based circuits can be modulated by a variety of target signaling agents, modulation of the circuit is not limited to electronic signaling agents. Nucleic acid sensor molecule-based circuits can respond to biologic target signaling molecules, for example neurotransmitters, hormones, proteins, and nucleic acids, that enable the integration of the nucleic acid circuits into biological systems.

In one embodiment, the nucleic acid sensor molecules (allozymes) are used to detect the presence of or absence of single stranded RNA (ssRNA) in a system, for example in a blood sample, cell extract, cell, or entire organism. An array of nucleic acid sensor molecules, for example when attached to a surface such as a chip or bead, can be used to detect and profile ssRNA in a system. As such, nucleic acid sensor molecules can be used in the analysis and/or profiling of gene expression in vitro or in vivo. The information generated by the nucleic acid sensor array can be used in mapping gene expression patterns and genotyping for various purposes, for example in target discovery, target validation, drug discovery, determining susceptibility to disease, determining the potential effect of various treatments or therapies, predicting drug metabolism or drug response, selecting candidates for clinical trials, and for managing the treatment of disease in individual patients.

In another embodiment, the nucleic acid sensor molecules (allozymes) are used to detect the presence of or absence of single nucleotide polymorphisms (SNPs) or single stranded DNA (ssDNA) in a system, for example in a blood sample, cell extract, cell, or entire organism. An array of nucleic acid sensor molecules, for example when attached to a surface such as a chip or bead, can be used to detect and profile SNPs or ssDNA in a system. As such, nucleic acid sensor molecules can be used in SNP discovery, detection, and scoring. In a non-limiting example, a plurality of nucleic acid sensor molecules is used to screen a fetus, infant, child or adult for genetic defects based on the SNP profile of the fetus, infant, child or adult. A sample of genetic material is obtained from, for example amniotic fluid, chorionic villus, blood, or hair and is contacted with an array of nucleic acid sensor molecules. The array of nucleic acid sensor molecules comprises a SNP library such that the presence of any predetermined SNP is indicated by the corresponding nucleic acid sensor by measuring the extent of the signal produced when the nucleic acid sensor interacts with the SNP, for example by measuring fluorescence, color change, precipitate deposition, voltage or current. For example, a nucleic acid computer device of the invention can be integrated into the nucleic acid sensor array such that the output of the array is recorded electronically and can be subsequently downloaded into a database. An individual SNP profile, for example, a list of particular SNPs comprising a genotype, is established from the signals generated by the nucleic acid sensor array. As such, treatment of the fetus, infant, child or adult can be initiated before symptoms arise.

In another embodiment, the information generated by the nucleic acid sensor array can be used in genotyping for various purposes, for example in target discovery, target validation, drug discovery, determining susceptibility to disease, determining the potential effect of various treatments or therapies, predicting drug metabolism or drug response, selecting candidates for clinical trials, and for managing the treatment of disease in individual patients.

In another embodiment, the nucleic acid sensor molecules (allozymes) are used to detect the presence of or absence peptides and/or proteins in a system, for example in a blood sample, cell extract, cell, or entire organism. These nucleic acid molecules can be used in place of Elisa or Western Blot analysis, and provide a broader array of criteria to differentiate proteins and peptides in vivo. The nucleic acid sensor molecules can be used to differentiate proteins or peptides that differ in sequence, conformation, activation state or phosphorylation state, or by other post-translational modifications. An array of nucleic acid sensor molecules, for example when attached to a surface such as a chip or bead, can be used to detect and profile peptides and/or proteins in a system. As such, nucleic acid sensor molecules can be used in proteome discovery, detection, and scoring. In a non-limiting example, a plurality of nucleic acid sensor molecule is used to screen a fetus, infant, child or adult's proteome. A sample of genetic material is obtained from, for example amniotic fluid, chorionic villus, blood, or hair and is contacted with an array of nucleic acid sensor molecules. The array of nucleic acid sensor molecules comprises a proteome library such that the presence of any predetermined peptide or protein is indicated by the corresponding nucleic acid sensor by measuring the extent of the signal produced when the nucleic acid sensor interacts with the peptide or protein, for example by measuring fluorescence, color change, precipitate deposition, voltage or current. For example, a nucleic acid computer device of the invention can be integrated into the nucleic acid sensor array such that the output of the array is recorded electronically and can be subsequently downloaded into a database. The information generated by the nucleic acid sensor array can be used in diagnostic molecular profiling applications such as protein mapping or profiling for various purposes, for example in target discovery, target validation, drug discovery, determining susceptibility to disease, determining the potential effect of various treatments or therapies, predicting drug metabolism or drug response, selecting candidates for clinical trials, and for managing the treatment of disease in individual patients.

In one embodiment, the nucleic acid sensor molecules (allozymes) of the invention are used for in vivo applications, for example in vivo ELISA, drug screening, and gene regulation. In vivo ELISA is essentially equivalent to western blot analysis. An allozyme specific to analyte, for example DNA, RNA, protein, small molecule, metabolite etc., can be constitutively expressed along with green fluorescent protein (GFP). The allozyme is designed such that when activated it cleaves GFP mRNA thus inhibiting GFP expression. In the presence of an analyte, the GFP signal would not be observed and in the absence of the analyte, full expression of GFP would be achieved. Thus, by monitoring GFP expression the analyte concentration (e.g. protein expression) can be calculated. Similarly in vivo drug screening can be achieved using a similar system. This system would give direct IC50 and EC50 values. In one embodiment, nucleic acid sensor molecule of the invention (allozymes) can be used to modulate gene expression and the expression of RNA and protein in vivo. These nucleic acid sensor molecules are designed to respond to a signaling agent, for example, a gene, SNP, mutant protein, wild-type protein, overexpressed protein, mutant RNA, wild-type RNA, compounds, metals, polymers, other molecules and/or drugs in a system., which in turn modulates the activity of the nucleic acid sensor molecule. In response to interaction with a predetermined signaling agent, the nucleic acid sensor molecule's activity is activated or inhibited such that the expression of a particular target is selectively down-regulated. The target can comprise a wild-type protein or RNA, mutant protein or RNA, and/or a predetermined cellular component that modulates gene expression or protein activity. In a specific example, nucleic acid sensor molecules that are activated by interaction with an RNA encoding a target protein are used as therapeutic agents in vivo. The presence of RNA encoding the target protein activates the nucleic acid sensor molecule that subsequently cleaves the RNA encoding the target protein, resulting in the inhibition of protein expression. In this manner, cells that express the target protein are selectively targeted for therapeutic activity.

In another non-limiting example, an allozyme can be activated by a predetermined protein, peptide, or mutant polypeptide that causes the allozyme to inhibit the expression of the gene encoding the protein, peptide, or mutant polypeptide, by, for example, cleaving RNA encoded by the gene. In this non-limiting example, the allozyme acts as a decoy to inhibit the function of the protein, peptide, or mutant polypeptide and also inhibit the expression of the protein, peptide, or mutant polypeptide once activated by the protein, peptide, or mutant polypeptide.

Several in vitro selection (evolution) strategies (Orgel, 1979, Proc. R. Soc. London, B 205, 435) have been used to evolve new nucleic acid catalysts capable of catalyzing cleavage and ligation of phosphodiester linkages (Joyce, 1989, Gene, 82, 83-87; Beaudry et al., 1992, Science 257, 635-641; Joyce, 1992, Scientific American 267, 90-97; Breaker et al., 1994, TIBTECH 12, 268; Bartel et al.,1993, Science 261:1411-1418; Szostak, 1993, TIBS 17, 89-93; Kumar et al., 1995, FASEB J, 9, 1183; Breaker, 1996, Curr. Op. Biotech., 7, 442; Santoro et al., 1997, Proc. Natl. Acad. Sci., 94, 4262; Tang et al., 1997, RNA 3, 914; Nakamaye & Eckstein, 1994, supra; Long & Uhlenbeck, 1994, supra; Ishizaka et al., 1995, supra; Vaish et al., 1997, Biochemistry 36, 6495; Kuwabara et al., 2000, Curr. Opin. Chem. Biol., 4, 669) all of these are incorporated by reference herein). Each can catalyze a series of reactions including the hydrolysis of phosphodiester bonds in trans (and thus can cleave other RNA molecules) under physiological conditions.

There are several classes of enzymatic nucleic acids that are presently known. Each can catalyze the hydrolysis of RNA phosphodiester bonds in trans (and thus can cleave other RNA molecules) under physiological conditions. Table I summarizes some of the characteristics of a class of enzymatic nucleic acids known as ribozymes. In general, enzymatic nucleic acids act by first binding to a target. Such binding occurs, for example, through the interaction of the target RNA with one or more target binding portions of the enzymatic nucleic acid, wherein the target RNA and substrate binding portion complex is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its function, such as its ability to direct synthesis of an encoded protein. After an enzymatic nucleic acid has bound and cleaved its RNA target, it is released from that RNA to search for another target and can repeatedly bind and cleave new targets. Thus, a single enzymatic nucleic acid molecule is able to cleave many molecules of target RNA. In addition, the enzymatic nucleic acid molecule is a highly specific inhibitor of gene expression, with the specificity of inhibition depending not only on the base-pairing mechanism of binding to the target RNA, but also on the mechanism of target RNA cleavage. Single mismatches, or base-substitutions, near the site of cleavage can completely eliminate catalytic activity of an enzymatic nucleic acid.

In one of the preferred embodiments of the inventions described herein, the nucleic acid sensor molecule is formed based on a hammerhead or hairpin motif. In other preferred embodiments, the nucleic acid sensor molecule is formed in the motif of a hepatitis delta virus, group I intron, group II intron or RNase P RNA (in association with an RNA guide sequence), Neurospora VS RNA, DNAzymes, NCH cleaving motifs, or G-cleavers. Examples of such hammerhead motifs are described by Dreyfus, supra, Rossi et al., 1992, AIDS Research and Human Retroviruses 8, 183; of hairpin motifs by Hampel et al., EP0360257, Hampel and Tritz, 1989 Biochemistry 28, 4929, Feldstein et al., 1989, Gene 82, 53, Haseloff and Gerlach, 1989, Gene, 82, 43, and Hampel et al., 1990 Nucleic Acids Res. 18, 299; Chowrira & McSwiggen, U.S. Pat. No. 5,631,359; of the hepatitis delta virus motif is described by Perrotta and Been, 1992 Biochemistry 31, 16; of the RNase P motif by Guerrier-Takada et al., 1983 Cell 35, 849; Forster and Altman, 1990, Science 249, 783; Li and Altman, 1996, Nucleic Acids Res. 24, 835; Neurospora VS RNA ribozyme motif is described by Collins (Saville and Collins, 1990 Cell 61, 685-696; Saville and Collins, 1991 Proc. Natl. Acad. Sci. USA 88, 8826-8830; Collins and Olive, 1993 Biochemistry 32, 2795-2799; Guo and Collins, 1995, EMBO. J 14, 363); Group II introns are described by Griffin et al., 1995, Chem. Biol. 2, 761; Michels and Pyle, 1995, Biochemistry 34, 2965; Pyle et al., International PCT Publication No. WO 96/22689; of the Group I intron by Cech et al., U.S. Pat. No. 4,987,071 and of DNAzymes by Usman et al., International PCT Publication No. WO 95/11304; Chartrand et al., 1995, NAR 23, 4092; Breaker et al., 1995, Chem. Bio. 2, 655; Santoro et al., 1997, PNAS 94, 4262, and Beigelman et al., International PCT publication No. WO 99/55857. NCH cleaving motifs are described in Ludwig & Sproat, International PCT Publication No. WO 98/58058; and G-cleavers are described in Kore et al., 1998, Nucleic Acids Research 26, 4116-4120 and Eckstein et al., International PCT Publication No. WO 99/16871. Additional motifs such as the Aptazyme (aptamer dependent ribozyme) (Breaker et al., WO 98/43993), Amberzyme (Class I motif; FIG. 2; Beigelman et al., U.S. Ser. No. 09/301,511) and Zinzyme (FIG. 3) (Beigelman et al., U.S. Ser. No. 09/301,511), all included by reference herein including drawings, can also be used in the present invention. These specific motifs are not limiting in the invention and those skilled in the art will recognize that all that is important in an nucleic acid sensor molecule with catalytic activity of this invention is that it has a specific substrate binding site which is complementary to one or more of the target nucleic acid regions, and that it have nucleotide sequences within or surrounding that substrate binding site which impart catalytic activity to the molecule (Cech et al., U.S. Pat. No. 4,987,071). Further, for nucleic acid sensor molecules, all that is important is that the molecule comprise sequence and structure that is able to interact with its substrate and catalyze a chemical reaction of interest.

Preferably, a nucleic acid molecule of the instant invention is between 13 and 500 nucleotides in length. For example, nucleic acid sensor molecules of the invention are preferably between 25 and 300 nucleotides in length, more preferably between 30 and 150 nucleotides in length, e.g., 34, 36, 38, 46, 47, 56, 65, 78, or 136 nucleotides in length. Exemplary DNAzymes of the invention are preferably between 15 and 400 nucleotides in length, more preferably between 25 and 150 nucleotides in length, e.g., 29, 30, 31, or 32 nucleotides in length (see for example Santoro et al., 1998, Biochemistry, 37, 13330-13342; Chartrand et al., 1995, Nucleic Acids Research, 23, 4092-4096). Those skilled in the art will recognize that all that is required is for the nucleic acid molecule to be of length and conformation sufficient and suitable for the nucleic acid molecule to catalyze a reaction contemplated herein. The length of the nucleic acid molecules of the instant invention are not limiting within the general limits stated.

In a preferred embodiment, the invention provides a method for producing a class of nucleic acid-based diagnostic agents that exhibit a high degree of specificity for the target signaling molecule.

In additional embodiments, the invention features a method of detecting target signaling molecules or signaling agents in both in vitro and in vivo applications. In vitro diagnostic applications can comprise both solid support based and solution based chip, multichip-array, micro-well plate, and micro-bead derived applications as are commonly used in the art. In vivo diagnostic applications can include but are not limited to cell culture and animal model based applications, comprising differential gene expression arrays, FACS based assays, diagnostic imaging, and others.

By “signaling agent” or “target signaling agent” is meant a chemical or physical entity capable of interacting with a nucleic acid sensor molecule, specifically a sensor component of a nucleic acid sensor molecule, resulting in modification of the enzymatic nucleic acid component of the nucleic acid sensor molecule via chemical, physical, topological, or conformational changes to the structure of the molecule such that the activity of the enzymatic nucleic acid component is modulated, for example is activated or deactivated. Signaling agents can comprise target signaling molecules such as macromolecules, ligands, small molecules, metals and ions, nucleic acid molecules including but not limited to RNA and DNA or analogs thereof, proteins, peptides, antibodies, polysaccharides, lipids, sugars, microbial or cellular metabolites, pharmaceuticals, and organic and inorganic molecules in a purified or unpurified form, or physical signals including magnetism, temperature, light, sound, shock, pH, capacitance, voltage, and ionic conditions.

By “enzymatic nucleic acid” is meant a nucleic acid molecule capable of catalyzing (altering the velocity and/or rate of) a variety of reactions including the ability to repeatedly cleave other separate nucleic acid molecules (endonuclease activity) or ligate other separate nucleic acid molecules (ligation activity) in a nucleotide base sequence-specific manner. Additional reactions amenable to nucleic acid sensor molecules include but are not limited to phosphorylation, dephosphorylation, isomerization, helicase activity, polymerization, transesterification, hydration, hydrolysis, alkylation, dealkylation, halogenation, dehalogenation, esterification, desterification, hydrogenation, dehydrogenation, saponification, desaponification, amination, deamination, acylation, deacylation, glycosylation, deglycosylation, silation, desilation, hydroboration, epoxidation, peroxidation, carboxylation, decarboxylation, substitution, elimination, oxidation, and reduction reactions on both small molecules and macromolecules. Such a molecule with endonuclease and/or ligation activity can have complementarity in a substrate binding region to a specified gene target, and also has an enzymatic activity that specifically cleaves and/or ligates RNA or DNA in that target. That is, the nucleic acid molecule with endonuclease and/or ligation activity is able to intramolecularly or intermolecularly cleave and/or ligate RNA or DNA and thereby inactivate or activate a target RNA or DNA molecule. This complementarity functions to allow sufficient hybridization of the enzymatic RNA molecule to the target RNA or DNA to allow the cleavage/ligation to occur. 100% complementarity is preferred, but complementarity as low as 50-75% can also be useful in this invention. In addition, nucleic acid sensor molecule can perform other reactions, including those mentioned above, selectively on both small molecule and macromolecular substrates, though specific interaction of the nucleic acid sensor molecule sequence with the desired substrate molecule via hydrogen bonding, electrostatic interactions, and Van der Waals interactions. The nucleic acids can be modified at the base, sugar, and/or phosphate groups. The term enzymatic nucleic acid is used interchangeably with phrases such as ribozymes, catalytic RNA, enzymatic RNA, catalytic DNA, catalytic oligonucleotides, nucleozyme, DNAzyme, RNA enzyme, endoribonuclease, endonuclease, minizyme, leadzyme, oligozyme, finderon or DNA enzyme. All of these terminologies describe nucleic acid molecules with enzymatic activity.

By “substrate binding arm” or “substrate binding domain” or “substrate binding region” is meant that portion or region of a nucleic acid sensor molecule which is able to interact, for example, via complementarity (i.e., able to base-pair with), with a portion of its substrate. Preferably, such complementarity is 100%, but can be less if desired. For example, as few as 10 bases out of 14 can be base-paired (see for example Werner and Uhlenbeck, 1995, Nucleic Acids Research, 23, 2092-2096; Hammann et al., 1999, Antisense and Nucleic Acid Drug Dev., 9, 25-31). Examples of such arms are shown generally in FIGS. 1-4. That is, these arms contain sequences within a nucleic acid sensor molecule which are intended to bring the nucleic acid sensor molecule and the target signaling molecule, for example RNA, together through complementary base-pairing interactions. The nucleic acid sensor molecule of the invention can have binding arms that are contiguous or non-contiguous and can be of varying lengths. The length of the binding arm(s) are preferably greater than or equal to four nucleotides and of sufficient length to stably interact with the target RNA. Preferably, the binding arm(s) are 12-100 nucleotides in length. More preferably, the binding arms are 14-24 nucleotides in length (see, for example, Werner and Uhlenbeck, supra; Hamman et al., supra; Hampel et al., EP0360257; Berzal-Herrance et al., 1993, EMBO J, 12, 2567-73). If two binding arms are chosen, the design is such that the length of the binding arms are symmetrical (i.e., each of the binding arms is of the same length; e.g., five and five nucleotides, or six and six nucleotides, or seven and seven nucleotides long) or asymmetrical (i.e., the binding arms are of different length; e.g., six and three nucleotides; three and six nucleotides long; four and five nucleotides long; four and six nucleotides long; four and seven nucleotides long; and the like).

By “enzymatic portion” or “catalytic domain” is meant that portion or region of the nucleic acid sensor molecule essential for catalyzing a chemical reaction, such as cleavage of a nucleic acid substrate.

By “system” is meant, material, in a purified or unpurified form, from biological or non-biological sources, including but not limited to human, animal, plant, bacteria, virus, fungi, soil, water, mechanical devices, circuits, networks, computers, or others that comprises the target signaling agent or target signaling molecule to be detected or amplified.

The “biological system” as used herein can be a eukaryotic system or a prokaryotic system, for example a bacterial cell, plant cell or a mammalian cell, or of plant origin, mammalian origin, yeast origin, Drosophila origin, or archebacterial origin.

By “reporter molecule” is meant a molecule, such as a nucleic acid sequence (e.g., RNA or DNA or analogs thereof) or peptides and/or other chemical moieties, able to stably interact with the nucleic acid sensor molecule and function as a substrate for the nucleic acid sensor molecule. The reporter molecule can also contain chemical moieties capable of generating a detectable response, including but not limited to, fluorescent, chromogenic, radioactive, enzymatic and/or chemiluminescent or other detectable labels that can then be detected using standard assays known in the art. The reporter molecule can also act as an intermediate in a chain of events, for example, by acting as an amplicon, inducer, promoter, or inhibitor of other events that can act as second messengers in a system.

In one embodiment, the reporter molecule of the invention is an oligonucleotide primer, template, or probe, which can be used to modulate the amplification of additional nucleic acid sequences, for example, sequences comprising reporter molecules, target signaling molecules, effector molecules, inhibitor molecules, and/or additional nucleic acid sensor molecules of the instant invention.

By “sensor component” of the nucleic acid sensor molecule is meant, a molecule such as a nucleic acid sequence (e.g., RNA or DNA or analogs thereof), peptide, or other chemical moiety which can interact with one or more regions of the enzymatic nucleic acid component of the nucleic acid sensor molecule to modulate, such as inhibit or activate, the catalytic activity of the nucleic acid sensor molecule. In the presence of a signaling agent, the ability of the sensor component, for example, to modulate the catalytic activity of the enzymatic nucleic acid component is inhibited or diminished. The sensor component can comprise recognition properties relating to chemical or physical signals capable of modulating the enzymatic nucleic acid component via chemical or physical changes to the structure of the nucleic acid sensor molecule. The sensor component can be derived from a naturally occurring nucleic acid protein binding sequence, for example RNAs that bind viral proteins such as HIV trans-activation response (TAR), HIV nucleocapsid, TFIIA, rev, rex, Ebola VP35, HCV core proteins, HBV core proteins; RNAs that bind eukaryotic proteins such as protein kinase R (PKR), ribosomal proteins, RNA polymerases, and ribonucleoproteins. The sensor component can also be derived from a nucleic acid sequence that is obtained through in vitro or in vivo selection techniques as are know in the art. Such sequences or “aptamers” can be designed to bind a specific protein, peptide, nucleic acid, co-factor, metabolite, drug, or other small molecule with varying affinity. The sensor component can be covalently linked to the nucleic acid sensor molecule, or can be non-covalently associated. A person skilled in the art will recognize that all that is required is that the sensor component is able to selectively inhibit the activity of the nucleic acid sensor molecule.

“Complementarity” refers to the ability of a nucleic acid to form hydrogen bond(s) with another RNA sequence by either traditional Watson-Crick or other non-traditional types. In reference to the nucleic molecules of the present invention, the binding free energy for a nucleic acid molecule with its target or complementary sequence is sufficient to allow the relevant function of the nucleic acid to proceed, e.g., enzymatic nucleic acid cleavage, ligation, isomerization, phosphorylation, or dephosphorylation. Determination of binding free energies for nucleic acid molecules is well known in the art (see, e.g., Turner et al., 1987, CSH Symp. Quant. Biol. LII pp.123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

By “alkyl” group is meant a saturated aliphatic hydrocarbon, including straight-chain, branched-chain, and cyclic alkyl groups. Preferably, the alkyl group has 1 to 12 carbons. More preferably it is a lower alkyl of from 1 to 7 carbons, more preferably 1 to 4 carbons. The alkyl group can be substituted or unsubstituted. When substituted the substituted group(s) are preferably, hydroxyl, cyano, alkoxy, ═O, ═S, NO₂ or N(CH₃)₂, amino, or SH. The term also includes alkenyl groups which are unsaturated hydrocarbon groups containing at least one carbon-carbon double bond, including straight-chain, branched-chain, and cyclic groups. Preferably, the alkenyl group has 1 to 12 carbons. More preferably it is a lower alkenyl of from 1 to 7 carbons, more preferably 1 to 4 carbons. The alkenyl group can be substituted or unsubstituted. When substituted the substituted group(s) can be preferably, hydroxyl, cyano, alkoxy, ═O, ═S, N₂, halogen, N(CH₃)₂, amino, or SH. The term “alkyl” also includes alkynyl groups which have an unsaturated hydrocarbon group containing at least one carbon-carbon triple bond, including straight-chain, branched-chain, and cyclic groups. Preferably, the alkynyl group has 1 to 12 carbons. More preferably it is a lower alkynyl of from 1 to 7 carbons, more preferably 1 to 4 carbons. The alkynyl group can be substituted or unsubstituted. When substituted the substituted group(s) is preferably, hydroxyl, cyano, alkoxy, ═O, ═S, NO₂ or N(CH₃)₂, amino or SH. Such alkyl groups can also include aryl, alkylaryl, carbocyclic aryl, heterocyclic aryl, amide and ester groups. An “aryl” group refers to an aromatic group which has at least one ring having a conjugated p electron system and includes carbocyclic aryl, heterocyclic aryl and biaryl groups, all of which can be optionally substituted. The preferred substituent(s) of aryl groups are halogen, trihalomethyl, hydroxyl, SH, OH, cyano, alkoxy, alkyl, alkenyl, alkynyl, and amino groups. An “alkylaryl” group refers to an alkyl group (as described above) covalently joined to an aryl group (as described above). Carbocyclic aryl groups are groups wherein the ring atoms on the aromatic ring are all carbon atoms. The carbon atoms are optionally substituted. Heterocyclic aryl groups are groups having from 1 to 3 heteroatoms as ring atoms in the aromatic ring and the remainder of the ring atoms are carbon atoms. Suitable heteroatoms include oxygen, sulfur, and nitrogen, and include furanyl, thienyl, pyridyl, pyrrolyl, N-lower alkyl pyrrolo, pyrimidyl, pyrazinyl, imidazolyl and the like, all optionally substituted. An “amide” refers to an —C(O)—NH—R, where R is either alkyl, aryl, alkylaryl or hydrogen. An “ester” refers to an —C(O)—OR′, where R is either alkyl, aryl, alkylaryl or hydrogen.

By “nucleotide” is meant a heterocyclic nitrogenous base in N-glycosidic linkage with a phosphorylated sugar. Nucleotides are recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein). There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleotide bases other than adenine, guanine, cytosine and uracil at 1′ position or their equivalents; such bases can be used at any position, for example, within the catalytic core of an nucleic acid sensor molecule and/or in the substrate-binding regions of the nucleic acid molecule.

By “nucleoside” is meant a heterocyclic nitrogenous base in N-glycosidic linkage with a sugar. Nucleosides are recognized in the art to include natural bases (standard), and modified bases well known in the art. Such bases are generally located at the 1′ position of a nucleoside sugar moiety. Nucleosides generally comprise a base and sugar group. The nucleosides can be unmodified or modified at the sugar, and/or base moiety, (also referred to interchangeably as nucleoside analogs, modified nucleosides, non-natural nucleosides, non-standard nucleosides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein). There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleoside bases other than adenine, guanine, cytosine and uracil at 1′ position or their equivalents; such bases can be used at any position, for example, within the catalytic core of an nucleic acid sensor molecule and/or in the substrate-binding regions of the nucleic acid molecule.

By “unmodified nucleotide” is meant a nucleotide with one of the bases adenine, cytosine, guanine, thymine, uracil joined to the 1′ carbon of beta-D-ribo-furanose.

By “modified nucleotide” is meant a nucleotide that contains a modification in the chemical structure of an unmodified nucleotide base, sugar and/or phosphate.

By “unmodified nucleoside” is meant a nucleoside with one of the bases adenine, cytosine, guanine, thymine, uracil joined to the 1′ carbon of beta-D-ribo-furanose.

By “modified nucleoside” is meant a nucleotide that contains a modification in the chemical structure of an unmodified nucleoside base or sugar.

By “Inozyme” or “NCH” motif is meant, an enzymatic nucleic acid molecule comprising a motif as is generally described as NCH Rz in FIG. 1. Inozymes possess endonuclease activity to cleave RNA substrates having a cleavage triplet NCN/, where N is a nucleotide, C is cytidine and H is adenosine, uridine or cytidine, and/represents the cleavage site. H is used interchangeably with X. Inozymes can also possess endonuclease activity to cleave RNA substrates having a cleavage triplet NCN/, where N is a nucleotide, C is cytidine, and/represents the cleavage site. “I” in FIG. 1 represents an Inosine nucleotide, preferably a ribo-Inosine or xylo-Inosine nucleoside

By “G-cleaver” motif is meant, an enzymatic nucleic acid molecule comprising a motif as is generally described as G-cleaver Rz in FIG. 1. G-cleavers possess endonuclease activity to cleave RNA substrates having a cleavage triplet NYN/, where N is a nucleotide, Y is uridine or cytidine and/represents the cleavage site. G-cleavers can be chemically modified as is generally shown in FIG. 1.

By “amberzyme” motif is meant, an enzymatic nucleic acid molecule comprising a motif as is generally described in FIG. 2. Amberzymes possess endonuclease activity to cleave RNA substrates having a cleavage triplet NG/N, where N is a nucleotide, G is guanosine, and/represents the cleavage site. Amberzymes can be chemically modified to increase nuclease stability through substitutions as are generally shown in FIG. 2. In addition, differing nucleoside and/or non-nucleoside linkers can be used to substitute the 5′-gaaa-3′ loops shown in the figure. Amberzymes represent a non-limiting example of an enzymatic nucleic acid molecule that does not require a ribonucleotide (2′-OH) group within its own nucleic acid sequence for activity.

By “zinzyme” motif is meant, an enzymatic nucleic acid molecule comprising a motif as is generally described in FIG. 3. Zinzymes possess endonuclease activity to cleave RNA substrates having a cleavage triplet including but not limited to YG/Y, where Y is uridine or cytidine, and G is guanosine and/represents the cleavage site. Zinzymes can be chemically modified to increase nuclease stability through substitutions as are generally shown in FIG. 3, including substituting 2′-O-methyl guanosine nucleotides for guanosine nucleotides. In addition, differing nucleotide and/or non-nucleotide linkers can be used to substitute the 5′-gaaa-2′ loop shown in the figure. Zinzymes represent a non-limiting example of an enzymatic nucleic acid molecule that does not require a ribonucleotide (2′-OH) group within its own nucleic acid sequence for activity.

By “DNAzyme” is meant, an enzymatic nucleic acid molecule that does not require the presence of a 2′-OH group within it for its activity. In particular embodiments the enzymatic nucleic acid molecule can have an attached linker(s) or other attached or associated groups, moieties, or chains containing one or more nucleotides with 2′-OH groups. DNAzymes can be synthesized chemically or expressed endogenously in vivo, by means of a single stranded DNA vector or equivalent thereof. An example of a DNAzyme is shown in FIG. 4 and is generally reviewed in Usman et al., International PCT Publication No. WO 95/11304; Chartrand et al., 1995, NAR 23, 4092; Breaker et al., 1995, Chem. Bio. 2, 655; Santoro et al., 1997, PNAS 94, 4262; Breaker, 1999, Nature Biotechnology, 17, 422-423; and Santoro et. al., 2000, J. Am. Chem. Soc., 122, 2433-39; Perrin et al., 2001, JACS., 123, 1556. Additional DNAzyme motifs can be selected for using techniques similar to those described in these references, and hence, are within the scope of the present invention.

By “sufficient length” is meant an oligonucleotide of greater than or equal to 3 nucleotides in length and long enough to provide the intended function (such as binding) under the expected condition. For example, for binding arms of a nucleic acid sensor molecule, “sufficient length” means that the binding arm sequence is long enough to provide stable binding to a target site under the expected reaction conditions and environment. Preferably, the binding arms are not so long as to prevent useful turnover of the nucleic acid molecule

By “stably interact” is meant interaction of the oligonucleotides with target nucleic acid (e.g., by forming hydrogen bonds with complementary nucleotides in the target under physiological conditions) that is sufficient to the intended purpose (e.g., cleavage of target RNA by an enzyme).

By “nucleic acid molecule” as used herein is meant a molecule comprising nucleotides. The nucleic acid can be single, double, or multiple stranded and can comprise modified or unmodified nucleotides or non-nucleotides or various mixtures and combinations thereof. Nucleic acid molecules shall include oligonucleotides, ribozymes, DNAzymes, templates, and primers.

By “oligonucleotide” is meant a nucleic acid molecule comprising a stretch of three or more nucleotides.

In a preferred embodiment the linker region, when present in the nucleic acid sensor molecule and/or reporter molecule is further comprised of nucleotide, non-nucleotide chemical moieties or combinations thereof. Non-limiting examples of non-nucleotide chemical moieties can include ester, anhydride, amide, nitrile, and/or phosphate groups.

In another embodiment, the non-nucleotide linker is as defined herein. The term “non-nucleotide” as used herein include either abasic nucleotide, polyether, polyamine, polyamide, peptide, carbohydrate, lipid, or polyhydrocarbon compounds. Specific examples include those described by Seela and Kaiser, Nucleic Acids Res. 1990, 18:6353 and Nucleic Acids Res. 1987, 15:3113; Cload and Schepartz, J. Am. Chem. Soc. 1991, 113:6324; Richardson and Schepartz, J. Am. Chem. Soc. 1991, 113:5109; Ma et al., Nucleic Acids Res. 1993, 21:2585 and Biochemistry 1993, 32:1751; Durand et al., Nucleic Acids Res. 1990, 18:6353; McCurdy et al., Nucleosides & Nucleotides 1991, 10:287; Jschke et al., Tetrahedron Lett. 1993, 34:301; Ono et al., Biochemistry 1991, 30:9914; Arnold et al., International Publication No. W089/02439; Usman et al., International Publication No. WO 95/06731; Dudycz et al., International Publication No. WO 95/11910 and Ferentz and Verdine, J. Am. Chem. Soc. 1991, 113:4000, all hereby incorporated by reference herein. Thus, in a preferred embodiment, the invention features an nucleic acid sensor molecule of the invention having one or more non-nucleotide moieties, and having enzymatic activity to perform a chemical reaction, for example to cleave an RNA or DNA molecule.

By “cap structure” is meant chemical modifications which have been incorporated at either terminus of the oligonucleotide (see for example Wincott et al., WO 97/26270, incorporated by reference herein). These terminal modifications protect the nucleic acid molecule from exonuclease degradation, and can help in delivery and/or localization within a cell. The cap can be present at the 5′-terminus (5′-cap) or at the 3′-terminus (3′ -cap) or can be present on both termini. In non-limiting examples: the 5′-cap is selected from the group comprising inverted abasic residue (moiety), 4′, 5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′, 4′-seco nucleotide; acyclic 3,4-dihydroxybutyl nucleotide; acyclic 3,5-dihydroxypentyl nucleotide, 3′-3′-inverted nucleotide moiety; 3′-3-inverted abasic moiety; 3-2′-inverted nucleotide moiety; 3′-2′-inverted abasic moiety; 1,4-butanediol phosphate; 3′ -phosphoramidate; hexylphosphate; aminohexyl phosphate; 3′-phosphate; 3′ -phosphorothioate; phosphorodithloate; or bridging or non-bridging methylphosphonate moiety (for more details see Wincott et al., International PCT publication No. WO 97/26270, incorporated by reference herein). In yet another preferred embodiment the 3′ -cap is selected from a group comprising, 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4′-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Iyer, 1993, Tetrahedron 49, 1925; incorporated by reference herein).

By “abasic” or “abasic nucleotide” is meant sugar moieties lacking a base or having other chemical groups in place of a base at the 1′ position, (for more details see Wincott et al., International PCT publication No. WO 97/26270).

The term “non-nucleotide” refers to any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenine, guanine, cytosine, uracil or thymine. The terms “abasic” or “abasic nucleotide” are meant to include sugar moieties lacking a base or having other chemical groups in place of a base at the 1′ position, (for more details see Wincott et al., International PCT publication No. WO 97/26270).

By “RNA” is meant a molecule comprising at least one ribonucleotide residue. By “ribonucleotide” or “2′-OH” is meant a nucleotide with a hydroxyl group at the 2′ position of a β-D-ribo-furanose moiety.

By “patient” is meant an organism, which is a donor or recipient of explanted cells or the cells themselves. “Patient” also refers to an organism to which the nucleic acid molecules of the invention can be administered. Preferably, a patient is a mammal or mammalian cells. More preferably, a patient is a human or human cells.

By “enhanced enzymatic activity” is meant to include activity measured in cells and/or in vivo where the activity is a reflection of both the catalytic activity and the stability of the nucleic acid molecules of the invention. In this invention, the product of these properties can be increased in vivo compared to an all RNA enzymatic nucleic acid or all DNA enzyme. In some cases, the individual catalytic activity or stability of the nucleic acid molecule can be decreased (i.e., less than ten-fold), but the overall activity of the nucleic acid molecule is enhanced, in vivo.

By “nucleic acid circuit” or “nucleic acid-based circuit” is meant an electronic circuit comprising one or more nucleic acids or oligonucleotides.

By “nucleic acid computer” or “nucleic acid-based computer” is meant a computing device or system comprising one or more nucleic acids or oligonucleotides. The nucleic acid computer can be used to interface biological systems, control other devices, or can be utilized to solve problems and/or manipulate data. Furthermore, the nucleic acid computer may comprise nucleic acid circuits.

By “halfzyme” is meant an enzymatic nucleic acid molecule assembled from two or more nucleic acid components. The enzymatic nucleic acid in the halfzyme configuration is active when all the necessary components interact with each other. The halfzyme construct can be engineered to have a component lacking from the structure or sequence of the enzymatic nucleic acid molecule appropriate such that enzymatic activity is inhibited. In the presence of the target signaling agent, the required component for enzymatic activity is provided such that the halfzyme is catalytically active (see for example FIG. 29).

By “predetermined RNA molecule” is meant a particular RNA molecule of known sequence, such as a viral RNA, messenger RNA, transfer RNA, ribosomal RNA etc.

By “system” is meant a group of substances or components that can be collectively combined or identified. A system can comprise a biological system, for example an organism, cell, or components, extracts, and samples thereof. A system can further comprise an experimental or artificial system, where various substances or components are intentionally combined together.

By “detectable response” is meant a chemical or physical property that can be measured, including, but not limited to changes in temperature, pH, frequency, charge, capacitance, or changes in fluorescent, chromogenic, radioactive, enzymatic and/or chemiluminescent levels or properties that can then be detected using standard methods known in the art.

By “single stranded RNA” (ssRNA) is meant a naturally occurring or synthetic ribonucleic acid molecule comprising a linear single strand, for example a ssRNA can be a messenger RNA (mRNA), transfer RNA (tRNA), ribosomal RNA (rRNA) etc. of a gene.

By “single stranded DNA” (ssDNA) is meant a naturally occurring or synthetic deoxyribonucleic acid molecule comprising a linear single strand, for example, a ssDNA can be a sense or antisense gene sequence or EST (Expressed Sequence Tag).

By “predetermined target” is meant a signaling agent or target signaling agent that is chosen to interact with a nucleic acid sensor molecule to generate a detectable response.

By “validate a predetermined gene target” is meant to confirm that a particular gene is associated with a specific phenotype, disease, or biological function in a system. Once the relationship between a gene and its function or resulting phenotype is determined, the gene can be targeted to modulate the activity of the gene.

By “validate a predetermined RNA target” is meant to confirm that a particular RNA transcript of a gene or other RNA is associated with a specific phenotype, disease, or biological function in a system. Once the relationship between the RNA and its function or resulting phenotype is determined, the RNA can be targeted to modulate the activity of the RNA or the gene encoding the RNA.

By “validate a predetermined peptide target” is meant to confirm that a particular peptide is associated with a specific phenotype, disease, or biological function in a system. Once the relationship between the peptide and its function or resulting phenotype is determined, the peptide or RNA encoding the peptide can be targeted to modulate the activity of the peptide or the gene encoding the peptide.

By “validate a predetermined protein target” is meant to confirm that a particular protein is associated with a specific phenotype, disease, or biological function in a system. Once the relationship between the protein and its function or resulting phenotype is determined, the protein or RNA encoding the protein can be targeted to modulate the activity of the protein or the gene encoding the protein.

By “validate a predetermined SNP target” is meant to confirm that a particular SNP of a gene is associated with a specific phenotype, disease, or biological function in a system. Once the relationship between the SNP and its function, associated gene function, or resulting phenotype is determined, the SNP can be targeted to modulate the activity of the SNP or the gene associated with the SNP.

By “SNP scoring” is meant a process of identifying and measuring the presence of SNPs in a genome. SNP scoring can also refer to a system of ranking single nucleotide polymorphisms in terms of the relationship between a particular SNP and a certain disease state or drug response in an organism, for example a human. SNP scoring can be used in determining the genotype of an organism.

By “SNP” is meant a single nucleotide polymorphism as is known in the art to include single nucleotide substitutions or mismatches in a genome (see Brookes, 1999, Gene, 234, 177-186; Stephens, 1999, Molecular Diagnosis, 4, 309-317). SNPs can be used to identify genes and gene functions as well as to characterize a genotype.

By “proteome” is meant the complete set of proteins found in a particular system, such as a cell or organism, for example a human cell or human.

By “proteome map” is meant the functional relationship between different protein constituents of a proteome.

By “proteome scoring” is meant a process of identifying and measuring the presence of proteins in a proteome. Proteome scoring can also refer to a system of ranking proteins in terms of the relationship between a particular protein and a certain disease state or drug response in an organism, for example a human. Proteome scoring can be used in determining the phenotype of an organism.

By “disease specific proteome” is meant a proteome associated with a particular disease or condition.

By “treatment specific proteome” is meant a proteome associated with a particular treatment or therapy.

By “comprising” is meant including, but not limited to, whatever follows the word “comprising” . Thus, use of the term “comprising” indicates that the listed elements are required or mandatory, but that other elements are optional and can or can not be present. By “consisting of” is meant including, and limited to, whatever follows the phrase “consisting of”. Thus, the phrase “consisting of” indicates that the listed elements are required or mandatory, and that no other elements can be present.

Other features and advantages of the invention will be apparent from the following description of the preferred embodiments thereof, and from the claims.

### DESCRIPTION OF THE PREFERRED EMBODIMENTS

The drawings will first briefly be described.

### DRAWINGS

FIG. 1 shows examples of chemically stabilized ribozyme motifs. HH Rz, represents hammerhead ribozyme motif (Usman et al., 1996, Curr. Op. Struct. Bio., 1, 527); NCH Rz represents the NCH ribozyme motif (Ludwig & Sproat, International PCT Publication No. WO 98/58058); G-Cleaver, represents G-cleaver ribozyme motif (Kore et al., 1998, Nucleic Acids Research 26, 4116-4120). N or n, represent independently a nucleotide which can be same or different and have complementarity to each other; rI, represents ribo-Inosine nucleotide; arrow indicates the site of cleavage within the target. Position 4 of the HH Rz and the NCH Rz is shown as having 2′-C-allyl modification, but those skilled in the art will recognize that this position can be modified with other modifications well known in the art, so long as such modifications do not significantly inhibit the activity of the ribozyme.

FIG. 2 shows an example of the Amberzyme ribozyme motif that is chemically stabilized (see, for example, Beigelman et al., International PCT publication No. WO 99/55857, incorporated by reference herein; also referred to as Class I Motif). The Amberzyme motif is a class of enzymatic nucleic molecules that do not require the presence of a ribonucleotide (2′-OH) group for its activity.

FIG. 3 shows an example of the Zinzyme A ribozyme motif that is chemically stabilized (Beigelman et al., International PCT publication No. WO 99/55857, incorporated by reference herein; also referred to as Class A or Class II Motif). The Zinzyme motif is a class of enzymatic nucleic molecules that do not require the presence of a ribonucleotide (2′-OH) group for its activity.

FIG. 4 shows an example of a DNAzyme motif described by Santoro et al., 1997, PNAS, 94, 4262.

FIG. 5 shows a non-limiting example of the detection of a target sequence using a hammerhead-based cis-blocking sequence strategy. In this case, the enzymatic nucleic acid component is the nucleic acid sensor molecule, and in the absence of target, is inactivated by intramolecular folding. Addition of target sequence allows interaction of the sensor molecule/target complex to the reporter sequence to allow cleavage of the reporter molecule by the activated target/sensor molecule complex, providing a fluorescent signal due to the separation of flurophore and quench molecules. This same concept can be applied to other enzymatic nucleic acid components of the instant invention, including but not limited to Inozymes, G-cleavers, DNAzymes, Zinzymes, Amberzymes, and Hairpins. In addition, the configuration of the blocking sequence can hybridize with a variety of sequence positions both in cis and in trans (e.g., intermolecular binding and/or intramolecular binding) and in a variety of different locations on the sensor molecule. Additional non-limiting configurations are summarized in FIGS. 7-9.

FIG. 6 shows a schematic diagram indicating the two primary configurations of a cis-acting nucleic acid sensor molecule. The molecule can be either bound to a target sequence (A) or unbound and therefore bound to itself (B).

FIG. 7 displays a number of potential secondary structures for the nucleic acid sensor molecules in non-limiting examples. Schemes A-D show the activation of a nucleic acid sensor molecule by target sequence binding to a sensor component of the nucleic acid sensor molecule enabling catalysis, for example cleavage, of a reporter molecule.

FIG. 8 displays a number of potential secondary structures for the nucleic acid sensor molecules in non-limiting examples. Schemes E-H show the activation of a nucleic acid sensor molecule by target sequence binding to a sensor component of the nucleic acid sensor molecule enabling catalysis, for example cleavage, of a reporter molecule.

FIG. 9 displays a number of potential secondary structures for the nucleic acid sensor molecules in non-limiting examples. Schemes I and J show the activation of a nucleic acid sensor molecule by target sequence binding to a sensor component of the nucleic acid sensor molecule enabling catalysis, for example cleavage, of a reporter molecule.

FIG. 10 displays a number of potential secondary structures for the nucleic acid sensor molecules in non-limiting examples. Scheme K shows the activation of a nucleic acid sensor molecule by target sequence binding to a sensor component of the nucleic acid sensor molecule enabling catalysis, for example cleavage, of a reporter molecule.

FIG. 11 displays a number of potential secondary structures for the nucleic acid sensor molecules in non-limiting examples. Schemes M and N show the activation of a nucleic acid sensor molecule by target sequence binding to a peptide sensor component of the nucleic acid sensor molecule enabling catalysis, for example cleavage, of a reporter molecule.

FIG. 12 displays a number of potential secondary structures for the nucleic acid sensor molecules in non-limiting examples. Schemes O and P show the activation of a nucleic acid sensor molecule by target sequence binding to a sensor component of the nucleic acid sensor molecule enabling catalysis, for example cleavage, of a reporter molecule.

FIG. 13 displays a number of potential secondary structures for the nucleic acid sensor molecules in non-limiting examples. Schemes Q and R show the activation of a nucleic acid sensor molecule by target sequence binding to a sensor component of the nucleic acid sensor molecule enabling catalysis, for example cleavage, of a reporter molecule.

FIG. 14 displays the inherent amplification capacity of the diagnostic system of the instant invention. Due to the catalytic nature of the nucleic acid sensor molecule, catalysis can take place on a multitude of reporter molecules, resulting in an amplified signal in response to a single target signaling molecule.

FIG. 15 shows the structure of a diagnostic system of the instant invention, comprising a nucleic acid sensor molecule and a separate sensor component.

FIG. 16 is a bar graph that shows the results of testing nucleic acid sensor molecule/sensor component combinations in a cleavage assay. The reporter molecules were 5′-end labeled with ³²P-phosphate and incubated for 12 or 60 minutes in either: (1) buffer alone (50 mM Tris, pH 7.5, 10 mM MgCl₂), or in the presence of (2) 10 nM nucleic acid sensor molecule, (3) 10 nM nucleic acid sensor molecule plus 20 nM sensor component, (4) 10 nM nucleic acid sensor molecule plus 200 nM sensor component, or (5) 10 nM nucleic acid sensor molecule plus 20 nM sensor component and 500 nM target signaling molecule. At the end of the incubation the reactions were loaded onto a PAGE gel to separate cleaved reporter from uncleaved reporter. The gel was imaged on a Molecular Dynamics phosphorimager and quantitated to determine the percent of reporter cleaved under each set of conditions. Control reactions were carried out to ensure that addition of sensor component or target signaling molecule sequence, without nucleic acid sensor molecule, did not result in reporter cleavage; only 0.2-0.4% of reporter was cleaved under these conditions.

FIGS. 17a-c are a schematic representation of the method of the invention used to isolate nucleic acid sensor molecules capable of autoligation reactions useful in a variety of applications, including diagnostic applications. FIG. 17a shows the general selection scheme used for isolating active sequences. A random pool of nucleic acid sequence, such as RNA is combined with a substrate molecule comprising the structure R1-L-R2-Biotin, wherein R1 is selected from the group consisting of methyl, hydrogen, phosphate, nucleoside, nucleotide, oligonucleotide, R2 is selected from the group comprising molecules capable of generating a detectable signal, such as molecular beacons, small molecules, fluorophores, chemophores, ionophores, radio-isotopes, photophores, peptides, proteins, enzymes, antibodies, nucleic acids, and enzymatic nucleic acids, L represents a linker which can be present or absent, and “—” represents a covalent bond. Catalytically active sequences are biotinylated. The reaction mixture is passed over a solid support derivatized with Avidin, resulting in the capture of the biotinylated, catalytically active sequence pool. The support bound sequences are amplified by methods known in the art. FIG. 17b shows the selection of the initial pool of sequences that provide ligation activity, and subsequent selection of molecules that are active in the presence of a ligand of diagnostic interest. Initially, selection of catalytic sequences takes place in the absence of the ligand of diagnostic interest. The active molecules isolated from the first round of selection that initially bind to the Avidin derivatized support are eliminated. Molecules that pass through the support are re-selected in the presence of the ligand of diagnostic interest. The re-selected pool that binds to the support after reaction in the presence of the ligand of diagnostic interest is amplified by methods known in the art and transcribed for subsequent rounds of selection. FIG. 17c shows another selection strategy for isolating nucleic acid molecules capable of autoligation in the presence of a ligand of diagnostic interest. In this case, an initial selection takes place in the absence of the ligand to select sequences with autoligation activity. This pool is mutagenized by methods known in the art. The resulting mutagenized pool is selected for ligand binding activity by methods known in the art, for example, by using ligand affinity chromatography or gel shift assays. The resulting pool is mutagenized by methods known in the art. The original selection (for activity) is repeated in the presence of the ligand of diagnostic interest, with counterselection for molecules that react in the absence of the ligand.

FIGS. 18a-c are a schematic representation of the method of the invention used to isolate nucleic acid sensor molecules capable of isomerization reactions that have applications in a variety of fields, including diagnostics. R1 and R2 represent compounds, which can be the same or different, capable of generating a detectable signal or quenching a detectable signal when an isomerization event takes place, comprising molecular beacons, small molecules, fluorophores, chemophores, ionophores, radio-isotopes, photophores, peptides, proteins, enzymes, antibodies, nucleic acids, and enzymatic nucleic acids, L represents a linker which can be present or absent, and “—” represents a covalent bond. FIG. 18a shows the general selection scheme used for isolating active sequences. A random pool of nucleic acid sequences are passed over the complex of interest, derivatized to a solid support. The representative example of the complex shown in the figure consists of two fluorescent molecules joined together via a cis-carbon double bond linkage. Alternatively, a trans-carbon double bond linkage can be used. The selection pool is enriched and mutagenized throughout multiple generations to generate a diverse pool of “cis” binding sequences. Cis-binding nucleic acid molecules are then loaded onto the resin and the corresponding trans isomer of the complex is used to elute sequences that bind the trans-isomer tighter than the cis-isomer. FIG. 18b shows how the concentration of cis-isomer on the resin and the concentration of trans-isomer eluant can be manipulated in order to select sequences that prefer binding to one isomer over the other, and can therefor drive the reaction in the desired direction. FIG. 18c shows a selection scheme for isolating signaling agent dependent nucleic acid isomerase molecules from the initial selection pool from FIG. 18a . A counter-selection takes place in which sequences that are bound to the cis-isomer complex are eluted with a signaling agent of interest An additional counter-selection takes place in which sequences that are bound to the cis-isomer complex are eluted with the signaling agent of interest. A selection then takes place in which sequences remaining from the counter-selection rounds that are bound to the cis-isomer complex are eluted with a mixture of the signaling agent of interest and the trans-isomer complex, the eluted ligand dependent nucleic acid catalyst sequences are amplified and transcribed by methods known in the art.

FIG. 19 shows non-limiting examples of a Zinzyme sensor molecule. In the example provided, in the presence of a target signaling molecule (SEQ ID NO. 25), for example the stem-loop III region of Hepatitis C virus (HCV) (SEQ ID NO. 26), the sensor molecule adopts a conformation that cleaves a tagged reporter molecule, for example in trans (SEQ ID NO. 22) providing cleave of the reporter molecule at 5′-NYG/N-3′, where / represents the cleavage site, N represents any nucleotide, Y represents any pyrimidine nucleotide, and G represents Guanosine. Alternatively, a sensor molecule/reporter molecule complex, for example (SEQ ID NO. 27), provides cleavage of a tagged reporter molecule in cis, resulting in the release of the Tag, for example Tag-AGAAC. The Tag can comprise beacons, small molecules, fluorophores, chemophores, ionophores, radio-isotopes, photophores, peptides, proteins, enzymes, antibodies, nucleic acids, and enzymatic nucleic acids.

FIG. 20 shows non-limiting examples of Zinzyme sensor molecules. In the examples provided, in the presence of a target protein signaling molecule, for example a Hepatitis C virus (HCV) core protein, the sensor molecule, for example (SEQ ID NO. 28, SEQ ID NO. 29) adopts a conformation that cleaves a tagged reporter molecule, for example in trans (SEQ ID NO. 22) providing cleave of the reporter molecule at 5′-NYG/N-3′, where / represents the cleavage site, N represents any nucleotide, Y represents any pyrimidine nucleotide, and G represents Guanosine. Alternatively, a sensor molecule/reporter molecule complex provides cleavage of a tagged reporter molecule in cis, resulting in the release of the Tag, for example Tag-NNNN. The Tag can comprise beacons, small molecules, fluorophores, chemophores, ionophores, radio-isotopes, photophores, peptides, proteins, enzymes, antibodies, nucleic acids, and enzymatic nucleic acids.

FIG. 21 shows a non-limiting example of the attachment of a high turnover protein enzyme, for example luciferase, horseradish peroxidase, beta-galactosidase, or alkaline phosphatase as a reporter molecule of a nucleic acid sensor molecule of the invention, for example a Zinzyme sensor molecule. The coupling chemistry used to attach the enzyme to the 3′-end of an oligonucleotide comprises oxidation of a cis diol (that can be present in a nucleotide or non-nucleotide moiety, for example an abasic derivative) followed by conjugation of a free amine of the protein or linker conjugated to the protein and reduction with sodium borohydride. Alternately, R is a phosphoramidite moiety, wherein a protein enzyme conjugated nucleoside or abasic moiety is coupled to the 5′-end of an oligonucleotide.

FIG. 22 shows a non-limiting example of the use of a high turnover protein enzyme, for example luciferase, horseradish peroxidase, beta-galactosidase, or alkaline phosphatase, used as a component of a reporter molecule in conjunction with a nucleic acid sensor molecule. A system comprising a solution phase and a solid phase is used, wherein a biotin conjugated Zinzyme sensor molecule is used to detect the presence of a target signaling molecule (for example SEQ ID NO 31). In the presence of a target signaling molecule (“target” in the figure), the reporter molecule component of the sensor molecule is released from the sensor molecule when the sensor molecule interacts with the target signaling molecule in solution. The solution phase components are passed through a solid phase derivatized with avidin, streptavidin, or neutravidin. The eluent is assayed to indicate the presence of the high turnover enzyme by providing substrate for the enzyme. Enzyme activity is indicative of the presence of the target signaling molecule in the system. Alternatively, the sensor molecule is attached to a solid support, for example covalently, wherein a sample is passed through or is passed over the support bound sensor molecule. The eluent is assayed to indicate the presence of the high turnover enzyme by providing substrate for the enzyme. Enzyme activity is indicative of the presence of the target signaling molecule in the system.

FIG. 23 shows a non-limiting example of a ligase sensor molecule with an enzymatic reporter component. The system shown comprises a sensor molecule covalently attached to a surface and a separate reporter molecule, for example a high turnover protein enzyme reporter molecule which can be attached to a nucleic acid molecule, or is optionally not attached to a nucleic acid molecule. Other reporter molecules can be used in the system, including but not limited to beacons, small molecules, fluorophores, chemophores, ionophores, radio-isotopes, photophores, peptides, proteins, antibodies, nucleic acids, and enzymatic nucleic acids. In the presence of a target signaling molecule (“target” in the figure), the nucleic acid sensor molecule interacts with the target signaling molecule under conditions suitable for the ligation of the reporter molecule to the nucleic acid sensor molecule. The surface is washed to remove any unbound reporter molecules and is subsequently assayed for the presence of the reporter molecule, for example by providing a substrate for a high turnover protein enzyme reporter molecule and measuring conversion of the substrate.

FIG. 24 shows a non-limiting example of a scheme for selecting and utilizing a ligase sensor molecule of the invention such as the construct described in FIG. 23. The sensor molecule is selected from a motif comprising three regions including a 5′-constant region that is used as a template for polymerase activity, a variable region (for example 50 nucleotides), and a 3′-constant region that comprises the target signaling molecule to be detected by the sensor molecule. A restriction enzyme cleavage site is introduce between the 3′-constant region and the variable region of the construct. Furthermore, a biotin conjugated reporter molecule, for example selected from the group comprising beacons, small molecules, fluorophores, chemophores, ionophores, radio-isotopes, photophores, peptides, proteins, antibodies, nucleic acids, and enzymatic nucleic acids, is used to isolate sensor molecules that are selected for the capability to ligate the reporter molecule. Once sensor molecules are identified that can ligate the reporter molecule, the 3′-constant region representing the target signaling molecule is cleaved from the construct. In a system, for example one in which the nucleic acid sensor molecule is attached to a surface, interaction of the target signaling molecule with the nucleic acid sensor molecule under conditions suitable for the ligation of the reporter molecule to the nucleic acid sensor molecule. The surface is washed to remove any unbound reporter molecules and is subsequently assayed for the presence of the reporter molecule, for example by providing a substrate for a high turnover protein enzyme reporter molecule and measuring conversion of the substrate.

FIG. 25 shows a non-limiting example of a process whereby a nucleic acid signaling molecule is used in a nucleic acid circuit. The nucleic acid sensor molecule shown in the figure can be used to open or close an electronic circuit. In response to a target signaling agent, for example current, the nucleic acid sensor molecule catalyzes a chemical reaction comprising ligation in response to a predetermined current or cleavage in response to a predetermined current. The nucleic acid circuit is thereby modulated between an open and a closed state based on the predetermined input current that is applied to the circuit. A plurality of such circuits that comprise nucleic acid sensor modulation can be used in a variety of electronic devices, and can substitute solid state or silicon-based circuits in such devices. For example, computer processors comprising a plurality of nucleic acid sensor molecule based-circuits can be used in a computer device. Open and closed nucleic acid sensor molecule based-circuits can be used to generate or respond to binary code, creating a readable output.

FIG. 26 shows a non limiting example of target signaling molecule inactivation of a zinzyme sensor molecule. In the absence of the target (SEQ ID NO. 31), the zinzyme sensor molecule (SEQ ID NO. 32) catalyzes the cleavage of a reporter molecule (SEQ ID NO. 33).

FIG. 27 shows a non-limiting example of target signaling molecule activation of a zinzyme sensor molecule. In the presence of the target (SEQ ID NO. 34), the zinzyme sensor molecule (SEQ ID NO. 35) catalyzes the cleavage of a reporter molecule (SEQ ID NO. 36).

FIG. 28 shows a non-limiting example of a nucleic acid sensor molecule that is modulated by a protein target signaling molecule, Erk. In the presence of the target protein (Erk), the nucleic acid sensor molecule (SEQ ID NO. 39) catalyzes the cleavage of a reporter molecule.

FIG. 29 shows a non-limiting example of a “half-zinzyme” nucleic acid sensor molecule that is modulated by the 5′-UTR of the Hepatitis C virus (HCV 5′-UTR). The figure shows both inactive and active forms of the zinzyme sensor molecule (SEQ ID NO. 42). In the presence of the target signaling oligonucleotide (SEQ ID NO. 43) which represents the stem loop IIIB of the HCV 5′-UTR, the zinzyme sensor demonstrates an activity increase of three logs in cleaving the reporter molecule component of the sensor molecule as shown in the graph (+oligo target) as compared to the sensor molecule in the absence of the target. In the presence of the full length 350 nt. HCV 5′-UTR, the zinzyme sensor molecule demonstrates an almost one log increase in activity in cleaving the reporter molecule component of the sensor molecule.

FIG. 30 A-B shows a non-limiting example of half-zinzyme nucleic acid sensor molecule mediated detection of the HCV genome. FIG. 30a shows the structure of the 5′-UTR of the HCV genome. The sequence shown is the sequence used as an oligonucleotide target for nucleic acid sensor molecule catalysis. The purine guanosine RIG cleavage site is boxed. FIG. 30b shows the results of a half-zinzyme activity assay in which the half-zinzyme was incubated either in the presence or absence of the oligonucleotide target, or in the presence of RNase-H pre-treated HCV 5′-UTR. Half-zinzyme activity is expressed relative to the level observed in the presence of model oligonucleotide. Reactions included a 1:1 molar ratio of target to halfzyme.

FIG. 31 A-B shows a non-limiting example of nucleic acid sensor molecule activation by the protein kinase ERK2. FIG. 31 a shows and assay where the ERK2 nucleic acid sensor molecule (black bars) was incubated either in the presence of ERK2, BSA, or in the absence of any protein and assayed for activity. An enzymatic nucleic acid molecule that lacks the ERK2 sensor region was similarly incubated and assayed for activity (grey bars). Activity is expressed as the rate of substrate RNA cleavage relative to the rate observed in the presence of ERK2. FIG. 31b shows a graph of ERK2 concentration dependence in with the concentration of ERK2 was varied as indicated in allozyme reactions. Activity is expressed as the rate of substrate RNA cleavage relative to the maximal rate observed.

FIG. 32 A-B shows a non-limiting example of nucleic acid sensor molecule specificity. FIG. 32a shows that the ERK2 nucleic acid sensor molecule is MAPK homolog specific. Equal amounts of the mitogen activated protein kinases ERK2, JNK, or P38 were included in reactions containing the ERK2 nucleic acid sensor molecule. Activity is expressed as the rate of substrate RNA cleavage relative to the rate observed in the presence of ERK2. FIG. 32b shows the specificity of the ERK2 nucleic acid sensor molecule for activated (phosphorylated) ERK2. An equal amount of unactivated ERK2 (solid circles) or phosphorylated (activated) ERK2 (open circles) was incubated with ERK2 nucleic acid sensor and substrate cleavage was monitored over time. A reaction performed in parallel lacked protein (squares).

FIG. 33 shows a non-limiting example of a nucleic acid sensor ligase molecule of the invention that responds to HCV RNA.

FIG. 34 shows a schematic view of the secondary structure of the HCV 5′UTR (Brown et al., 1992, Nucleic Acids Res., 20, 5041-45; Honda et al., 1999, J. Virol., 73, 1165-74). Major structural domains are indicated in bold.

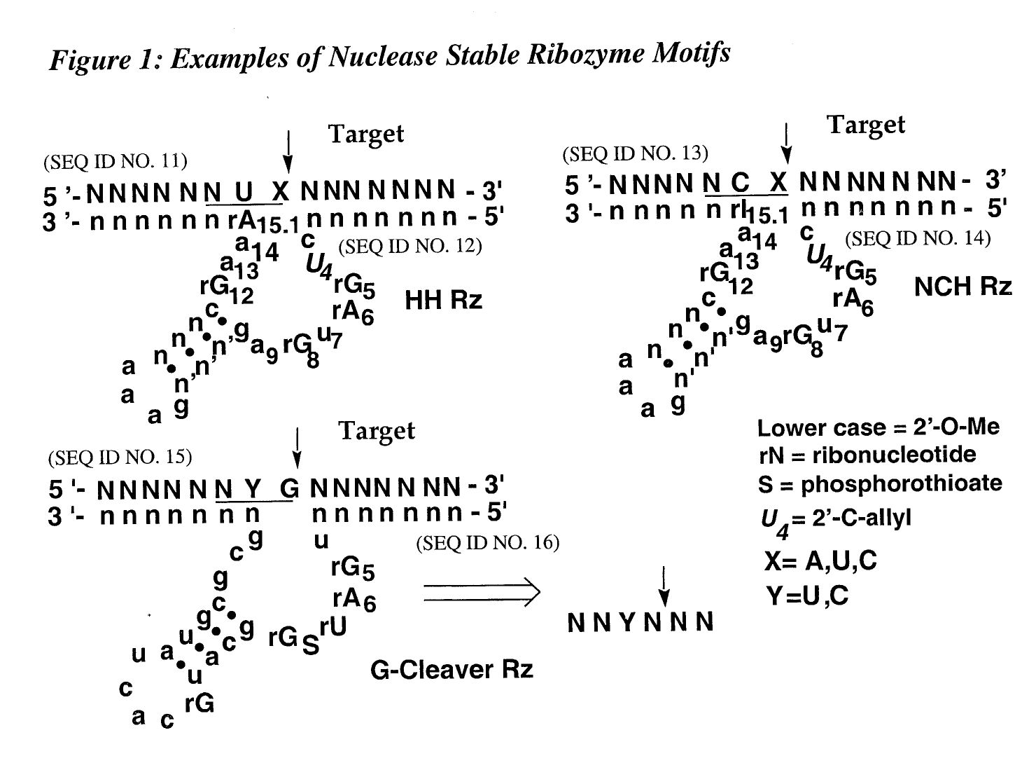

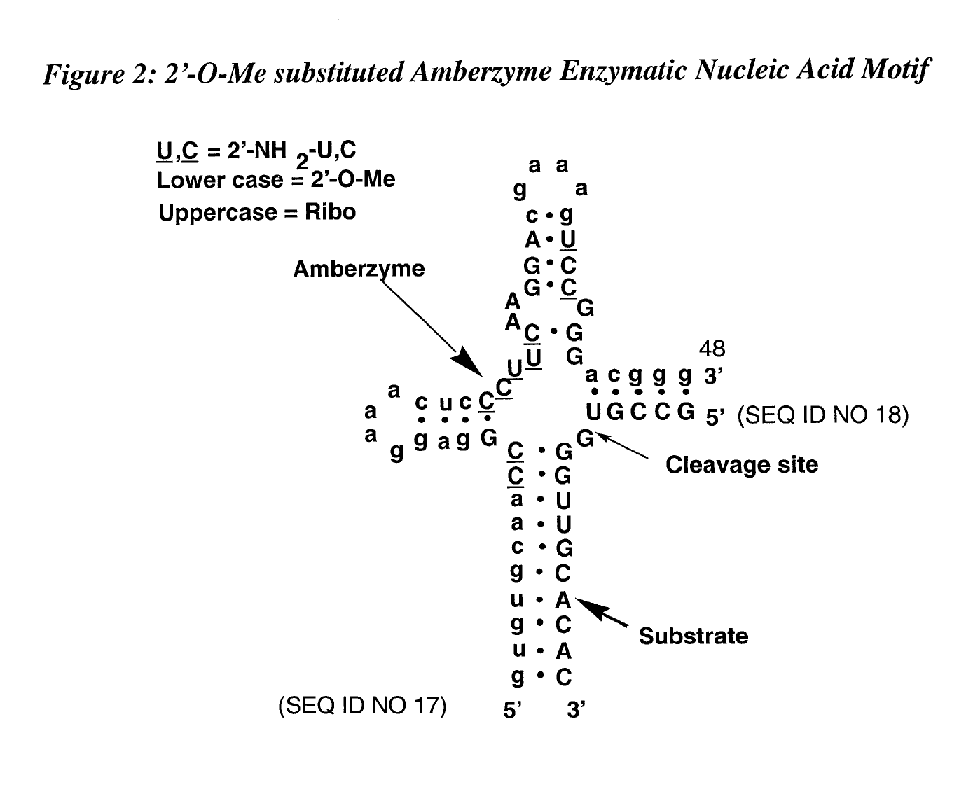

### DETECTION OF TARGET SIGNALING MOLECULES

In one embodiment, the invention features several approaches to detecting signaling agents, ligands and/or target signaling molecules in a system using nucleic acid molecules. In all cases, activity of the nucleic acid is modulated via interaction of the nucleic acid with the target signaling agent, ligand and/or target signaling molecule.

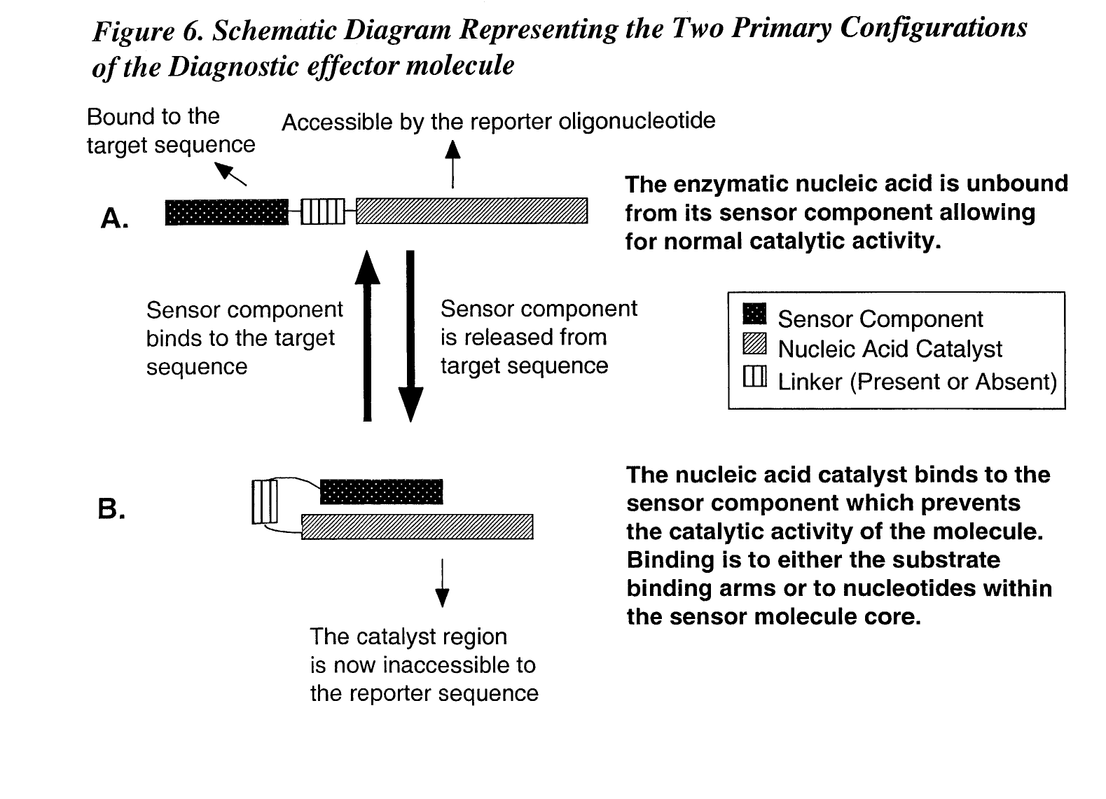

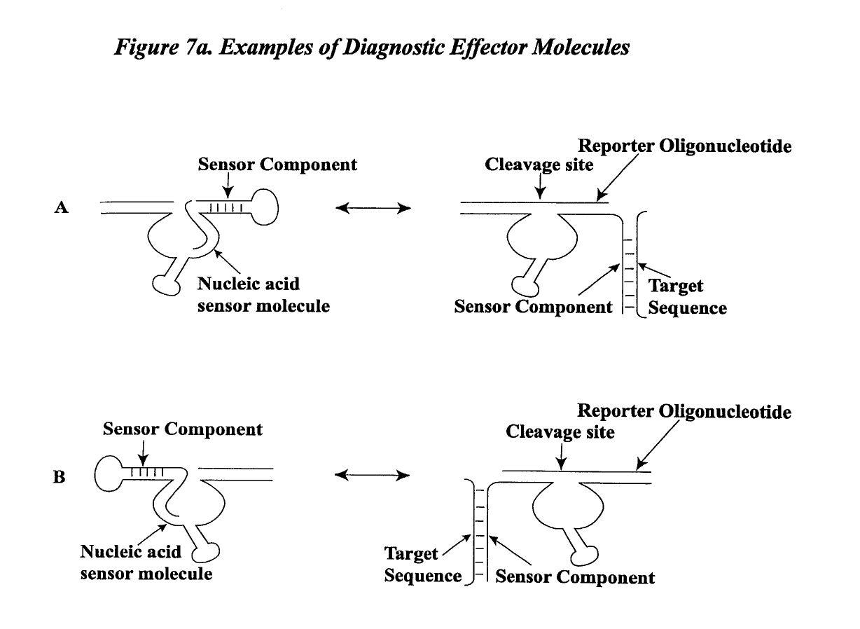

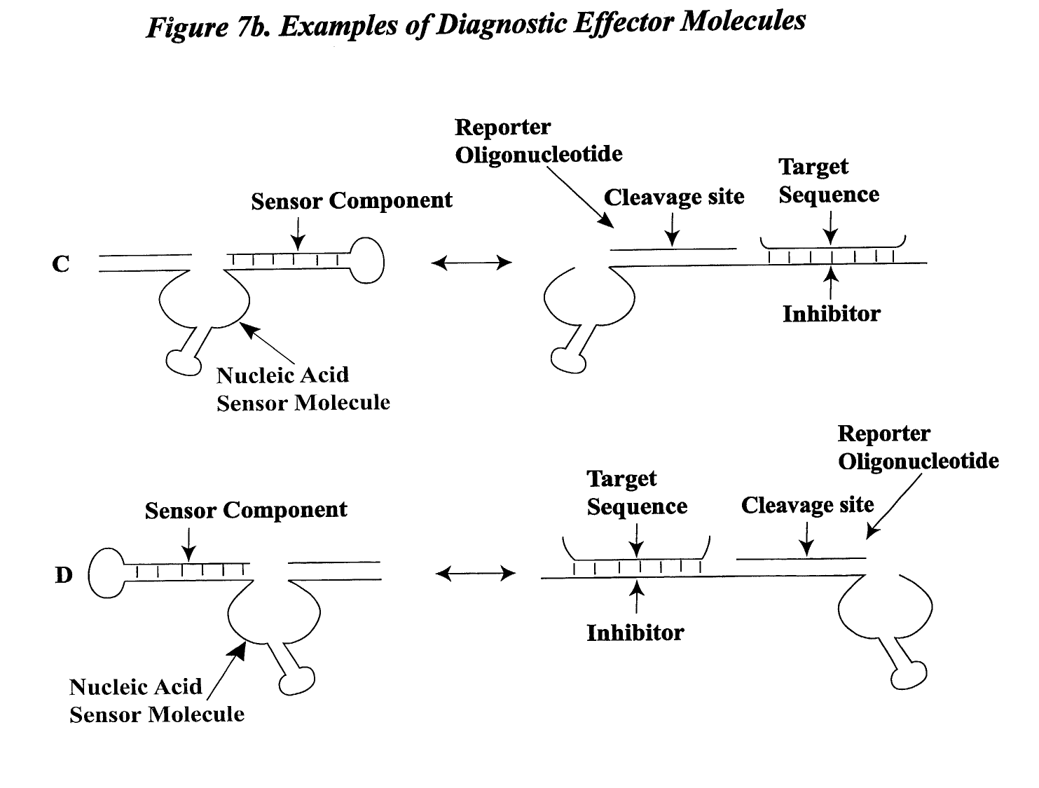

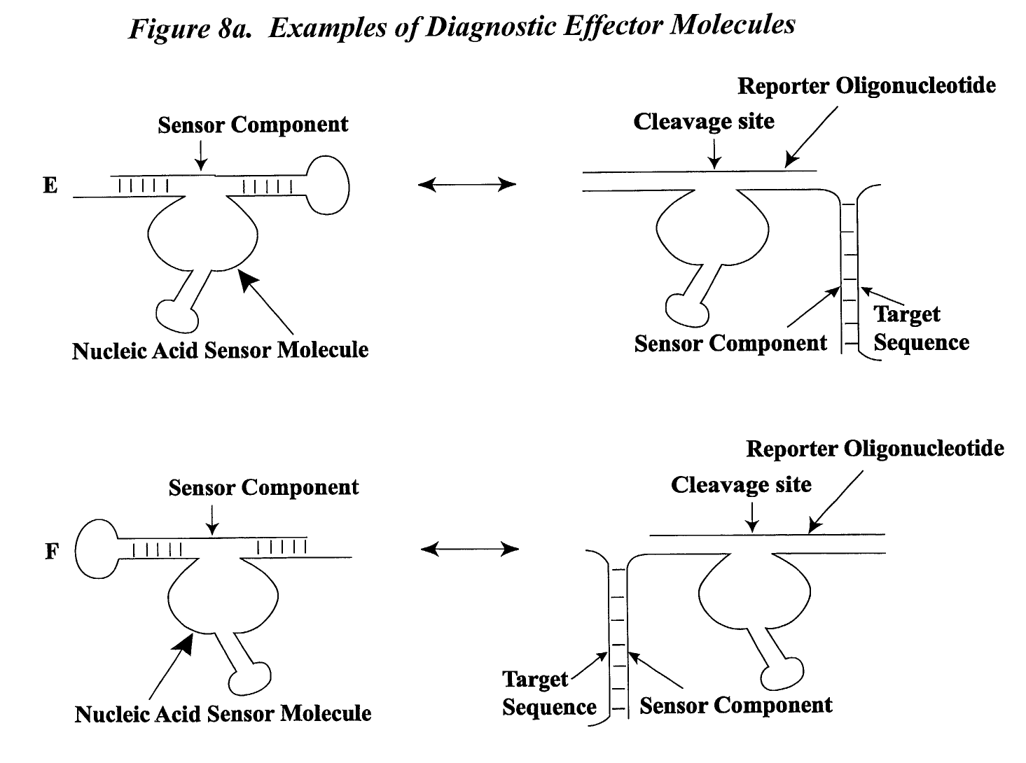

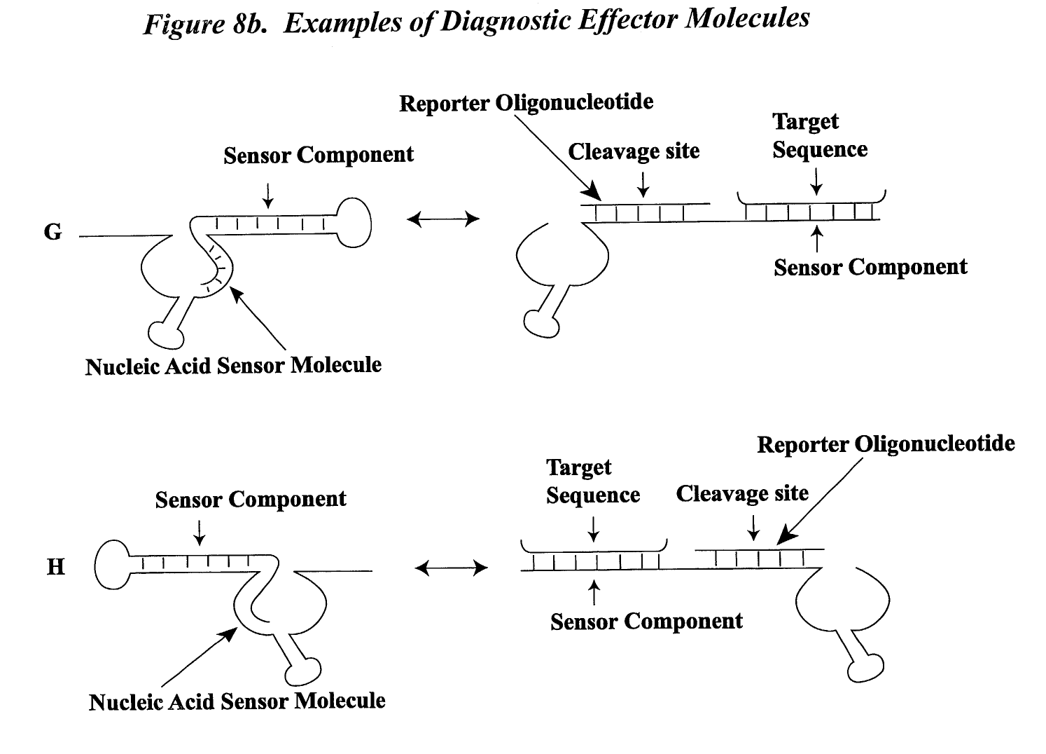

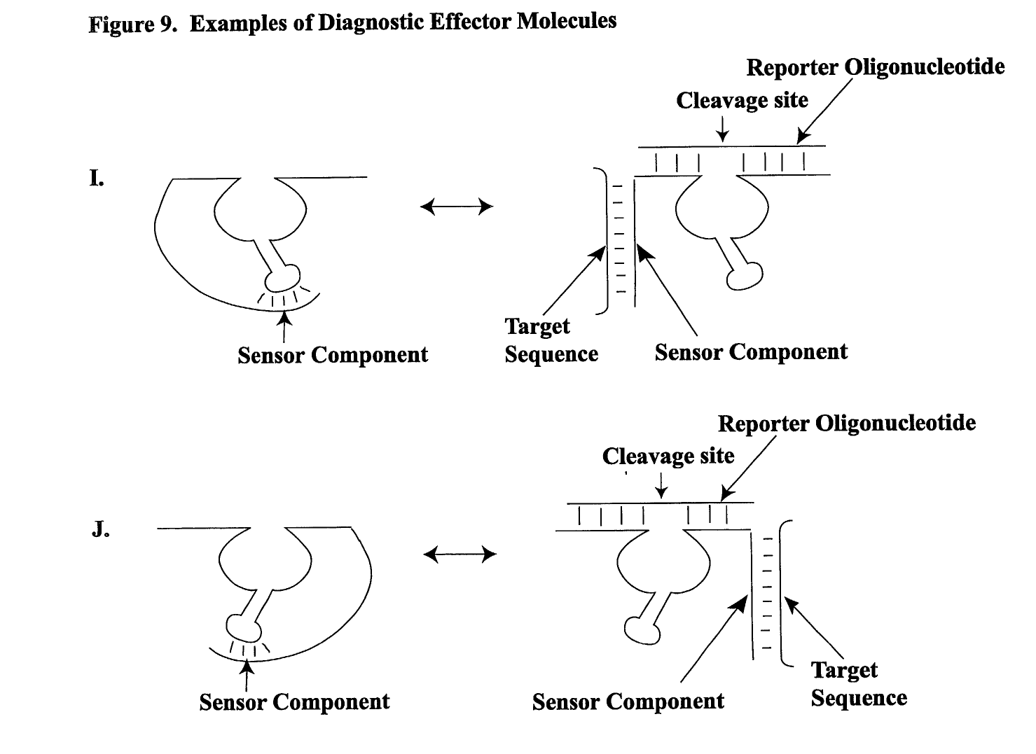

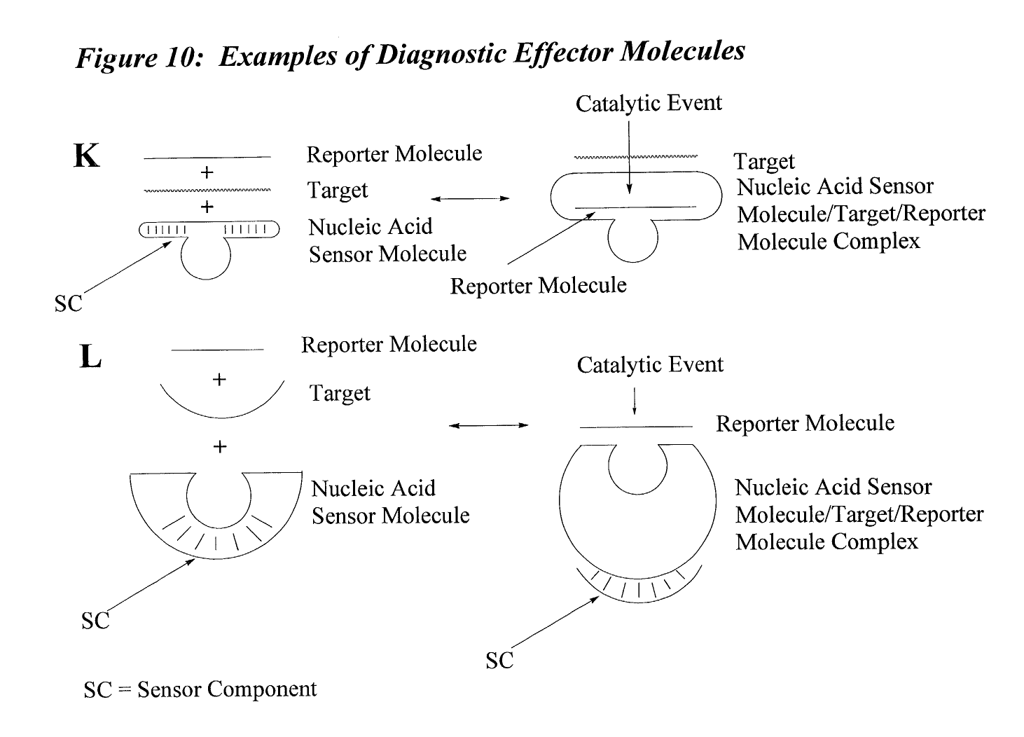

In one embodiment, the present invention utilizes at least three oligonucleotide sequences for proper function: nucleic acid sensor molecule, reporter molecule, and target signaling molecule. The nucleic acid sensor molecule is comprised of a sensor component, enzymatic nucleic acid component, and a linker between them which can be present or absent. The nucleic acid sensor molecule (FIG. 6), is in its inactive state when the sensor component binds to the nucleic acid sensor molecule in the enzymatic nucleic acid component. The sensor component can bind to the substrate binding regions or nucleotides that contribute to the secondary or tertiary structure of the enzymatic nucleic acid component. For example, the sensor component can bind to nucleotides located within the nucleic acid sensor molecule, which can disrupt catalytic activity. The reporter molecule can be able to bind to the nucleic acid sensor molecule, but a catalytic activity would be inhibited since the molecule is structurally inactive. Alternatively, the sensor component can bind to the substrate binding region(s) of the enzymatic nucleic acid component, which can prevent the reporter molecule from binding to the nucleic acid sensor molecule. The sensor component cannot be cleaved because the cleavage site would contain either a chemical modification which prevents cleavage or an inappropriate sequence. For example, hammerhead ribozymes need to have a NUH motif in the molecule to be cleaved (H is adenosine, cytidine, or uridine) for proper cleavage. By adding a guanosine at the H position in the RNA to be cleaved, cleavage can be inhibited.

In the presence of the target signaling molecule, the sensor component can disassociate from the enzymatic nucleic acid component and bind to the target signaling molecule preferentially. The sensor component can preferentially bind to the target signaling molecule which results in the formation of a more stable complex. For example, the sensor component can bind to more nucleotides on the target signaling molecule than on the nucleic acid sensor molecule. Binding to a larger number of nucleotides can have increased chemical stability and therefore is preferred over binding to a smaller number of nucleotides.

When the sensor component is bound to the target signaling molecule and the reporter molecule binds to the nucleic acid sensor molecule, a reaction can be catalyzed on the reporter molecule by the enzymatic nucleic acid component. For example, the reporter molecule can be cleaved. The cleavage event can then be detected by using a number of assays. For example, electrophoresis on a polyacrylamide gel would detect not only the full length reporter oligonucleotide but also any cleavage products that were created by the functional nucleic acid sensor molecule. The detection of these cleavage products indicate the presence of the target signaling molecule. In addition, the reporter molecule can contain a fluorescent molecule at one end which fluorescence signal is quenched by another molecule attached at the other end of the reporter molecule. Cleavage of the reporter molecule in this case results in the disassociation of the florescent molecule and the quench molecule, resulting in a signal. This signal can be detected and/or quantified by methods known in the art (for example see Nathan et al., U.S. Pat. No. 5,871,914, Birkenmeyer, U.S. Pat. No. 5,427,930, and Lizardi et al., U.S. Pat. No. 5,652,107, George et al., U.S. Pat. Nos. 5,834,186 and 5,741,679, and Shih et al., U.S. Pat. No. 5,589,332).

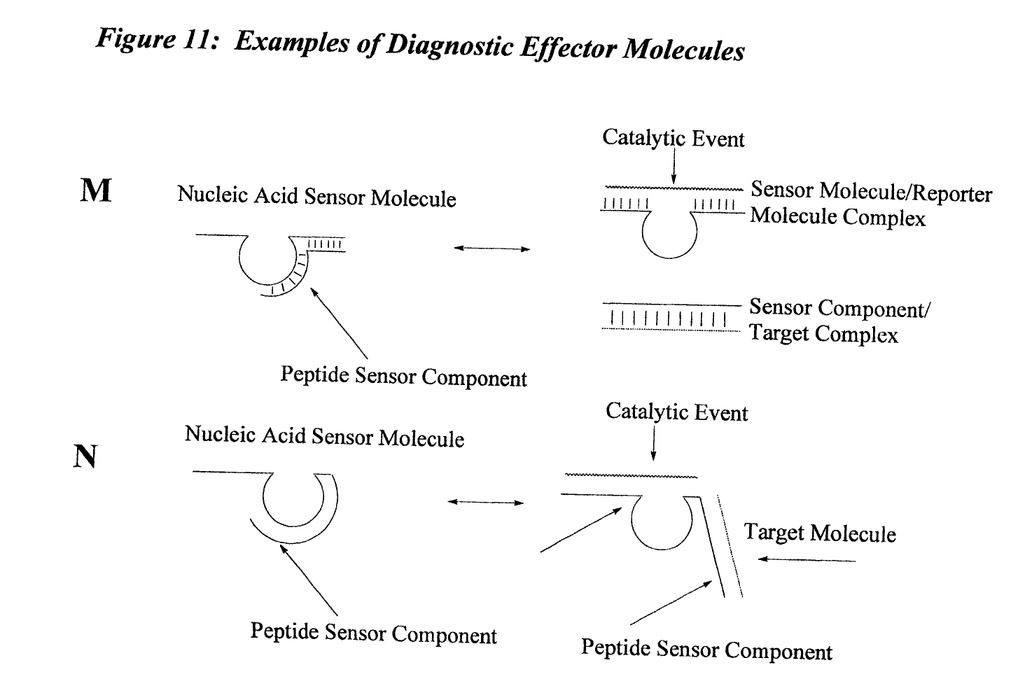

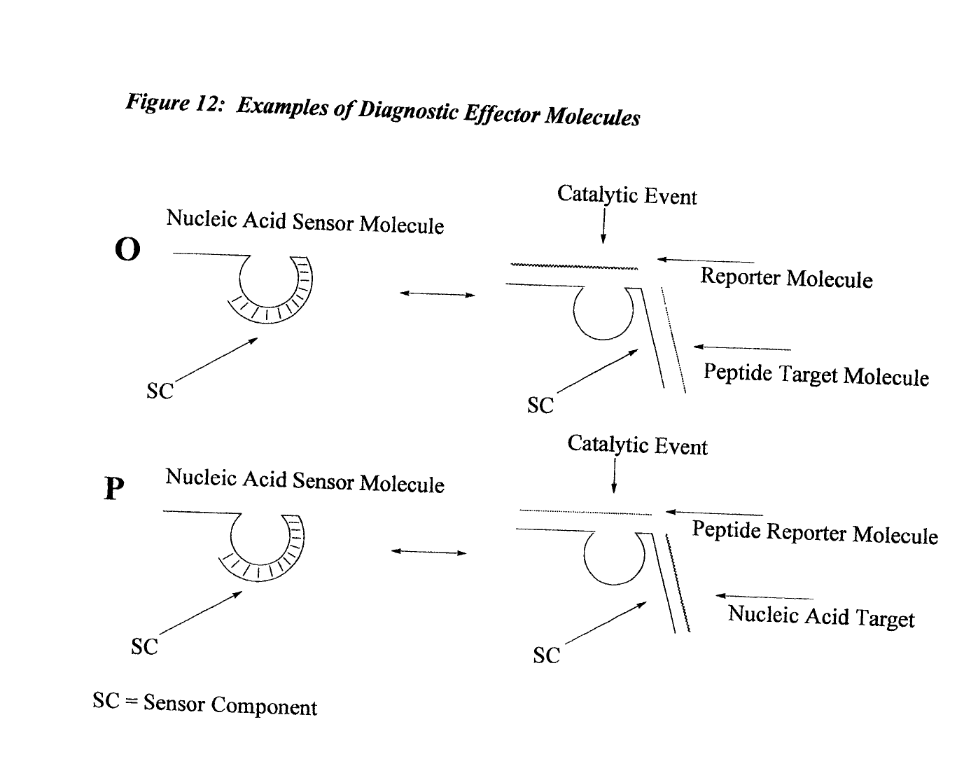

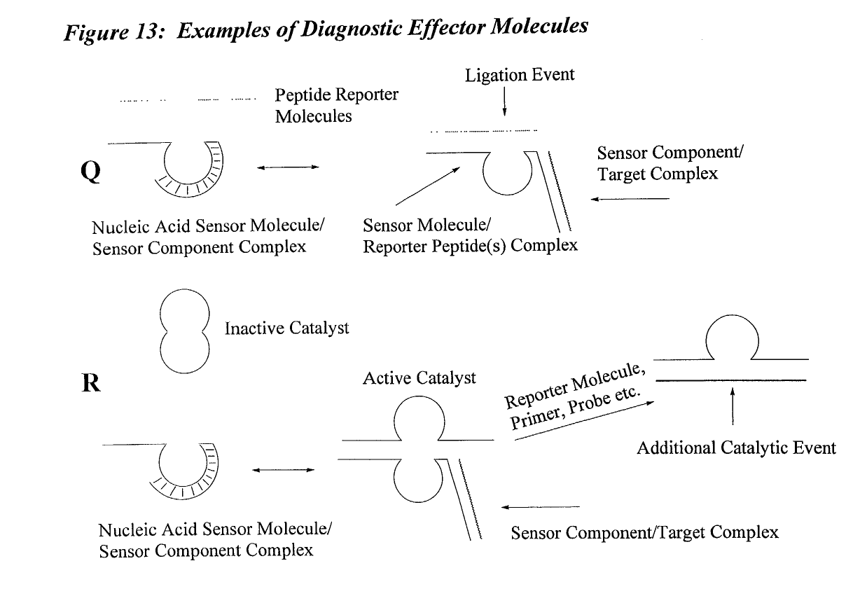

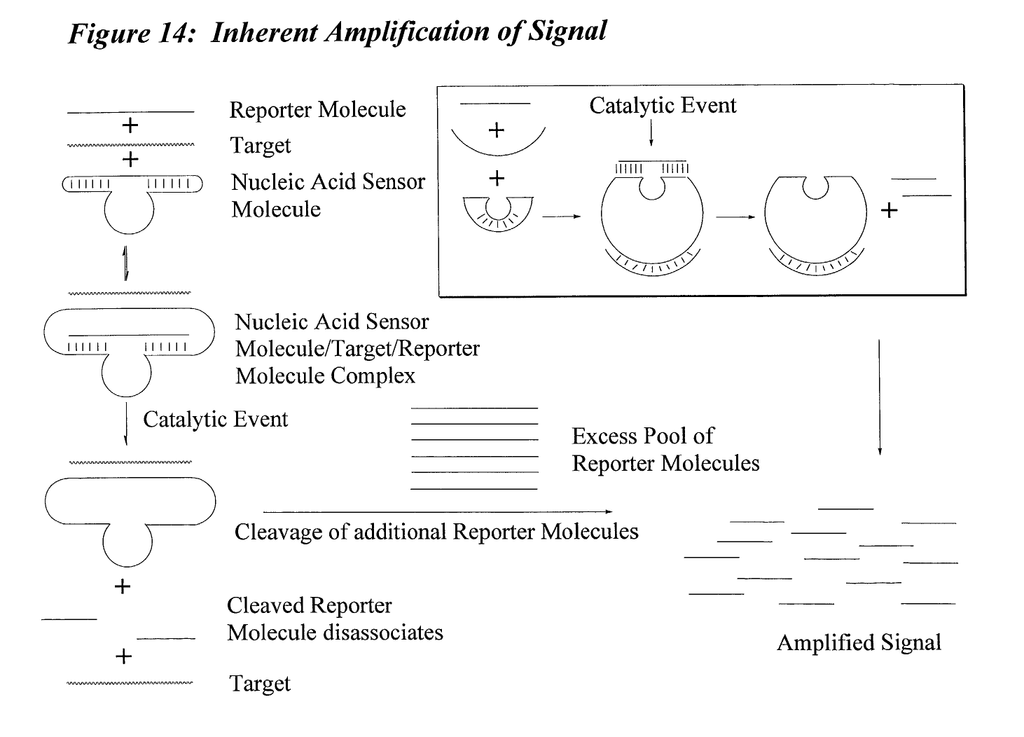

Alternatively, the sensor of the signaling molecule can comprise a separate oligonucleotide sequence, as shown for example in FIG. 11, system M.

### Target Sites

Targets for useful nucleic acid sensor molecules can be determined as disclosed in Draper et al., WO 93/23569; Sullivan et al., WO 93/23057; Thompson et al., WO 94/02595; Draper et al., WO 95/04818; McSwiggen et al., U.S. Pat. No. 5,525,468 and hereby incorporated by reference herein in totality. Rather than repeat the guidance provided in those documents here, below are provided specific examples of such methods, not limiting to those in the art. Nucleic acid sensor molecules to such targets are designed as described in those applications and synthesized to be tested in vitro and in vivo, as also described. Such nucleic acid sensor molecules can also be optimized and delivered as described therein.

Hammerhead, hairpin, Inozyme, Zinzyme, Amberzyme and DNAzyme-based nucleic acid sensor molecules are designed that can bind and are individually analyzed by computer folding (Jaeger et al., 1989 Proc. Natl. Acad. Sci. USA, 86, 7706; Denman, 1993, Biotechniques, 15, 1090) to assess whether the nucleic acid sensor molecule sequences fold into the appropriate secondary structure. Those nucleic acid sensor molecules with unfavorable intramolecular interactions between the binding arms and the catalytic core are eliminated from consideration. Varying binding arm lengths can be chosen to optimize activity. Generally, at least 5 bases on each arm are able to bind to, or otherwise interact with, the target RNA. Nucleic acid molecules of the differing motifs are designed to anneal to various sites in the mRNA message. The binding arms are complementary to the target site sequences described above.

Hammerhead, DNAzyme, NCH, amberzyme, zinzyme or G-Cleaver-based nucleic acid sensor molecule cleavage sites were identified and were designed to anneal to various sites in the RNA target. The binding arms are complementary to the target site sequences described above. The nucleic acid molecules were chemically synthesized. The method of synthesis used follows the procedure for normal DNA/RNA synthesis as described below and in Usman et al., 1987 J. Am. Chem. Soc., 109, 7845; Scaringe et al., 1990 Nucleic Acids Res., 18, 5433; Wincott et al., 1995 Nucleic Acids Res. 23, 2677-2684; and Caruthers et al., 1992, Methods in Enzymology 211,3-19.

### Nucleic Acid Molecule Synthesis

The nucleic acid molecules of the invention, including certain nucleic acid sensor molecules, can be synthesized using the methods described in Usman et al., 1987, J. Am. Chem. Soc., 109, 7845; Scaringe et al., 1990, Nucleic Acids Res., 18, 5433; and Wincott et al., 1995, Nucleic Acids Res. 23, 2677-2684 Wincott et al., 1997, Methods Mol. Bio., 74, 59. Such methods make use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 7.5 min coupling step for alkylsilyl protected nucleotides and a 2.5 min coupling step for 2′-O-methylated nucleotides. Table II outlines the amounts and the contact times of the reagents used in the synthesis cycle. Alternatively, syntheses at the 0.2 μmol scale can be done on a 96-well plate synthesizer, such as the PG2100 instrument produced by Protogene (Palo Alto, Calif.) with minimal modification to the cycle. A 33-fold excess (60 μL of 0.11 M =6.6 μmol) of 2′-O-methyl phosphoramidite and a 75-fold excess of S-ethyl tetrazole (60 μL of 0.25 M=15 μmol) can be used in each coupling cycle of 2′-O-methyl residues relative to polymer-bound 5′-hydroxyl. A 66-fold excess (120 μL of 0.11 M =13.2 μmol) of alkylsilyl (ribo) protected phosphoramidite and a 150-fold excess of S-ethyl tetrazole (120 μL of 0.25 M =30 μmol) can be used in each coupling cycle of ribo residues relative to polymer-bound 5′-hydroxyl. Average coupling yields on the 394 Applied Biosystems, Inc. synthesizer, determined by calorimetric quantitation of the trityl fractions, are typically 97.5-99%. Other oligonucleotide synthesis reagents for the 394 Applied Biosystems, Inc. synthesizer include; detritylation solution is 3% TCA in methylene chloride (ABI); capping is performed with 16% N-methyl imidazole in THF (ABI) and 10% acetic anhydride/10% 2,6-lutidine in THF (ABI); oxidation solution is 16.9 mM I₂, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide 0.05 M in acetonitrile) is used.

Cleavage from the solid support and deprotection of the oligonucleotide is typically performed using either a two-pot or one-pot protocol. For the two-pot protocol, the polymer-bound trityl-on oligoribonucleotide is transferred to a 4 mL glass screw top vial and suspended in a solution of 40% aq. methylamine (1 mL) at 65 ° C. for 10 min. After cooling to −20° C., the supernatant is removed from the polymer support. The support is washed three times with 1.0 mL of EtOH:MeCN:H2O/3:1:1, vortexed and the supernatant is then added to the first supernatant. The combined supernatants, containing the oligoribonucleotide, are dried to a white powder. The base deprotected oligoribonucleotide is resuspended in anhydrous TEA/HF/NMP solution (300 μL of a solution of 1.5 mL N-methylpyrrolidinone, 750 μL TEA and 1 mL TEA.3HF to provide a 1.4 M HF concentration) and heated to 65 ° C. After 1.5 h, the oligomer is quenched with 1.5 M NH₄HCO₃.

Alternatively, for the one-pot protocol, the polymer-bound trityl-on oligoribonucleotide is transferred to a 4 mL glass screw top vial and suspended in a solution of 33% ethanolic methylamine/DMSO: 1/1 (0.8 mL) at 65° C. for 15 min. The vial is brought to r.t. TEA.3HF (0.1 mL) is added and the vial is heated at 65 ° C. for 15 min. The sample is cooled at −20° C. and then quenched with 1.5 M NH₄HCO₃. An alternative deprotection cocktail for use in the one pot protocol comprises the use of aqueous methylamine (0.5 ml) at 65° C. for 15 min followed by DMSO (0.8 ml) and TEA.3HF (0.3 ml) at 65° C. for 15 min. A similar methodology can be employed with 96-well plate synthesis formats by using a Robbins Scientific Flex Chem block, in which the reagents are added for cleavage and deprotection of the oligonucleotide.

For anion exchange desalting of the deprotected oligomer, the TEAB solution is loaded onto a Qiagen 500® anion exchange cartridge (Qiagen Inc.) that is prewashed with 50 mM TEAB (10 mL). After washing the loaded cartridge with 50 mM TEAB (10 mL), the RNA is eluted with 2 M TEAB (10 mL) and dried down to a white powder.

For purification of the trityl-on oligomers, the quenched NH₄HCO₃ solution is loaded onto a C-18 containing cartridge that had been prewashed with acetonitrile followed by 50 mM TEAA. After washing the loaded cartridge with water, the RNA is detritylated with 0.5% TFA for 13 min. The cartridge is then washed again with water, salt exchanged with 1 M NaCl and washed with water again. The oligonucleotide is then eluted with 30% acetonitrile. Alternatively, for oligonucleotides synthesized in a 96-well format, the crude trityl-on oligonucleotide is purified using a 96-well solid phase extraction block packed with C18 material, on a Bahdan Automation workstation.

The average stepwise coupling yields are typically >98% (Wincott et al. 1995 Nucleic Acids Res. 23, 2677-2684). Those of ordinary skill in the art will recognize that the scale of synthesis can be adapted as larger or smaller than the example described above including but not limited to 96 well format, all that is important is the ratio of chemicals used in the reaction.

To ensure the quality of synthesis of nucleic acid molecules of the invention, quality control measures are utilized for the analysis of nucleic acid material. Capillary Gel Electrophoresis, for example using a Beckman MDQ CGE instrument, can be ulitized for rapid analysis of nucleic acid molecules, by introducing sample on the short end of the capillary. In addition, mass spectrometry, for example using a PE Biosystems Voyager-DE MALDI instrument, in combination with the Bohdan workstation, can be utilized in the analysis of oligonucleotides, including oligonucleotides synthesized in the 96-well format.

The nucleic acids of the invention can also be synthesized in two parts and annealed to reconstruct the nucleic acid sensor molecules (Chowrira and Burke, 1992 Nucleic Acids Res., 20, 2835-2840). The nucleic acids are also synthesized enzymatically using a variety of methods known in the art, for example as described in Havlina, International PCT publication No. WO 9967413, or from DNA templates using bacteriophage T7 RNA polymerase (Milligan and Uhlenbeck, 1989, Methods Enzymol. 180, 51). Other methods of enzymatic synthesis of the nucleic acid molecules of the invention are generally described in Kim et al., 1995, Biotechniques, 18, 992; Hoffman et al., 1994, Biotechniques, 17, 372; Cazenare et al., 1994, PNAS USA, 91, 6972; Hyman, U.S. Pat. No. 5,436,143; and Karpeisky et al., International PCT publication No. WO 98/28317)

Alternatively, the nucleic acid molecules of the present invention can be synthesized separately and joined together post-synthetically, for example by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204).

The nucleic acid molecules of the present invention are preferably modified to enhance stability by modification with nuclease resistant groups, for example, 2′-amino, 2′-C-allyl, 2′-flouro, 2′-O-methyl, 2′-H (for a review see Usman and Cedergren, 1992, TIBS 17, 34; Usman et al., 1994, Nucleic Acids Symp. Ser. 31, 163). Nucleic acid sensor molecules are purified by gel electrophoresis using known methods or are purified by high pressure liquid chromatography (HPLC; See Wincott et al., Supra, the totality of which is hereby incorporated herein by reference) and are re-suspended in water.

The sequences of the nucleic acids that are chemically synthesized, useful in this study, are shown in Table III. Those in the art will recognize that these sequences are representative only of many more such sequences where the enzymatic portion of the nucleic acid (all but the binding arms) is altered to affect activity. The nucleic acid construct sequences listed in Table III can be formed of ribonucleotides or other nucleotides or non-nucleotides. Such nucleic acids with enzymatic activity are equivalent to the nucleic acids described specifically in the Table.

### Optimizing Nucleic Acid Molecule Activity

Synthesizing nucleic acid molecules with modifications (base, sugar and/or phosphate) that prevent their degradation by serum ribonucleases can increase their potency (see e.g., Eckstein et al., International Publication No. W092/07065; Perrault et al., 1990 Nature 344, 565; Pieken et al., 1991, Science 253, 314; Usman and Cedergren, 1992, Trends in Bichem. Sci. 17, 334; Usman et al., International Publication No. WO93/15187; Rossi et al., International Publication No. WO 91/03162; Sproat, U.S. Pat. No. 5,334,711; and Burgin et al., supra; all of these describe various chemical modifications that can be made to the base, phosphate and/or sugar moieties of the nucleic acid molecules described herein. All these references are incorporated by reference herein. Modifications which enhance their efficacy in cells, and removal of bases from nucleic acid molecules to shorten oligonucleotide synthesis times and reduce chemical requirements are preferably desired.

There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups, for example, 2′-amino, 2′-C-allyl, 2′-flouro, 2′-O-methyl, 2′-H, nucleotide base modifications (for a review see Usman and Cedergren, 1992, TIBS. 17, 34; Usman et al., 1994, Nucleic Acids Symp. Ser. 31, 163; Burgin et al., 1996, Biochemistry , 35, 14090). Sugar modifications of nucleic acid molecules have been extensively described in the art (see Eckstein et al., International Publication PCT No. WO 92/07065; Perrault et al. Nature, 1990, 344, 565-568; Pieken et al. Science, 1991, 253, 314-317; Usman and Cedergren, Trends in Biochem. Sci. , 1992, 17, 334-339; Usman et al. International Publication PCT No. WO 93/15187; Sproat, U.S. Pat. No. 5,334,711 and Beigelman et al, 1995, J. Biol. Chem., 270, 25702; Beigelman et al., International PCT publication No. WO 97/26270; Beigelman et al., U.S. Pat. No. 5,716,824; Usman et al., U.S. Pat. No. 5,627,053; Woolf et al., International PCT Publication No. WO 98/13526; Thompson et al., U.S. Ser. No. 60/082,404 which was filed on Apr. 20, 1998; Karpeisky et al., 1998, Tetrahedron Lett., 39, 1131; Earnshaw and Gait, 1998, Biopolymers (Nucleic acid Sciences), 48, 39-55; Verma and Eckstein, 1998, Annu. Rev. Biochem., 67, 99-134; and Burlina et al., 1997, Bioorg. Med. Chem., 5, 1999-2010; all of the references are hereby incorporated by reference herein in their totalities). Such publications describe general methods and strategies to determine the location of incorporation of sugar, base and/or phosphate modifications and the like into nucleic acid sensor molecule molecules without inhibiting catalysis. In view of such teachings, similar modifications can be used as described herein to modify the nucleic acid molecules of the instant invention.

While chemical modification of oligonucleotide internucleotide linkages with phosphorothioate, phosphorothioate, and/or 5′-methylphosphonate linkages improves stability, many of these modifications can cause some toxicity. Therefore when designing nucleic acid molecules the amount of these internucleotide linkages should be minimized. The reduction in the concentration of these linkages should lower toxicity resulting in increased efficacy and higher specificity of these molecules.

Nucleic acid molecules having chemical modifications which maintain or enhance activity are provided. Such nucleic acid is also generally more resistant to nucleases than unmodified nucleic acid. Thus, in the presence of biological fluids, or in cells, the activity can not be significantly lowered. Clearly, nucleic acid molecules must be resistant to nucleases in order to function as effective diagnostic agents, whether utilized in vitro and/or in vivo. Improvements in the synthesis of RNA and DNA (Wincott et al., 1995 Nucleic Acids Res. 23, 2677; Caruthers et al., 1992, Methods in Enzymology 211,3-19; Karpeisky et al., International PCT publication No. WO 98/28317) (incorporated by reference herein) have expanded the ability to modify nucleic acid molecules by introducing nucleotide modifications to enhance their nuclease stability as described above.

In another aspect the nucleic acid molecules comprise a 5′ and/or a 3′-cap structure.

In one embodiment, the invention features modified nucleic acid molecules with phosphate backbone modifications comprising one or more phosphorothioate, phosphorodithioate, methylphosphonate, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications see Hunziker and Leumann, 1995, Nucleic Acid Analogues: Synthesis and Properties, in Modern Synthetic Methods, VCH, 331-417, and Mesmaeker et al., 1994, Novel Backbone Replacements for Oligonucleotides, in Carbohydrate Modifications in Antisense Research, ACS, 24-39. These references are hereby incorporated by reference herein.

In connection with 2′-modified nucleotides as described for the present invention, by “amino” is meant 2′—Nh₂ or 2′-O—NH₂, which can be modified or unmodified. Such modified groups are described, for example, in Eckstein et al., U.S. Pat. No. 5,672,695 and Karpeisky et al., WO 98/28317, respectively, which are both incorporated by reference herein in their entireties.

Various modifications to nucleic acid (e.g., nucleic acid sensor molecule) structure can be made to enhance the utility of these molecules. Such modifications enhance shelf-life, half-life in vitro, stability, and ease of introduction of such oligonucleotides to the target site, e.g., to enhance penetration of cellular membranes, and confer the ability to recognize and bind to targeted cells.

### Administration of Nucleic Acid Molecules

Methods for the delivery of nucleic acid molecules are described in Akhtar et al, 1992, Trends Cell Bio., 2, 139; and Delivery Strategies for Antisense Oligonucleotide Therapeutics, ed. Akhtar, 1995, which are both incorporated herein by reference. Sullivan et al., PCT WO 94/02595, further describes the general methods for delivery of enzymatic RNA molecules. These protocols can be utilized for the delivery of virtually any nucleic acid molecule. Nucleic acid molecules can be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. Alternatively, the nucleic acid/vehicle combination is locally delivered by direct injection or by use of an infusion pump. Other routes of delivery include, but are not limited to oral (tablet or pill form) and/or intrathecal delivery (Gold, 1997, Neuroscience, 76, 1153-1158). Other approaches include the use of various transport and carrier systems, for example though the use of conjugates and biodegradable polymers. For a comprehensive review on drug delivery strategies including CNS delivery, see Ho et al., 1999, Curr. Opin. Mol. Ther., 1, 336-343 and Jain, Drug Delivery Systems: Technologies and Commercial Opportunities, Decision Resources, 1998 and Groothuis et al., 1997, J. Neuro Virol., 3, 387-400. More detailed descriptions of nucleic acid delivery and administration are provided in Sullivan et al., supra, Draper et al, PCT WO93/23569, Beigelman et al., PCT WO99/05094, and Klimuk et al., PCT WO99/04819, all of which are incorporated by reference herein.

The molecules of the instant invention can be used as pharmaceutical agents. Pharmaceutical agents prevent, inhibit the occurrence, or treat (alleviate a symptom to some extent, preferably all of the symptoms) of a disease state in a patient.

The negatively charged polynucleotides of the invention can be administered (e.g., RNA, DNA or protein) and introduced into a patient by any standard means, with or without stabilizers, buffers, and the like, to form a pharmaceutical composition. When it is desired to use a liposome delivery mechanism, standard protocols for formation of liposomes can be followed. The compositions of the present invention can also be formulated and used as tablets, capsules or elixirs for oral administration; suppositories for rectal administration; sterile solutions; suspensions for injectable administration; and the other compositions known in the art.

The present invention also includes pharmaceutically acceptable formulations of the compounds described. These formulations include salts of the above compounds, e.g., acid addition salts, for example, salts of hydrochloric, hydrobromic, acetic acid, and benzene sulfonic acid.

A pharmacological composition or formulation refers to a composition or formulation in a form suitable for administration, e.g., systemic administration, into a cell or patient, preferably a human. Suitable forms, in part, depend upon the use or the route of entry, for example oral, transdermal, or by injection. Such forms should not prevent the composition or formulation from reaching a target cell (i.e., a cell to which the negatively charged polymer is desired to be delivered to). For example, pharmacological compositions injected into the blood stream should be soluble. Other factors are known in the art, and include considerations such as toxicity and forms which prevent the composition or formulation from exerting its effect.

By “systemic administration” is meant in vivo systemic absorption or accumulation of drugs in the blood stream followed by distribution throughout the entire body. Administration routes which lead to systemic absorption include, without limitations: intravenous, subcutaneous, intraperitoneal, inhalation, oral, intrapulmonary and intramuscular. Each of these administration routes expose the desired negatively charged polymers, e.g., nucleic acids, to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation which can facilitate the association of drug with the surface of cells, such as, lymphocytes and macrophages is also useful. This approach can provide enhanced delivery of the drug to target cells by taking advantage of the specificity of macrophage and lymphocyte immune recognition of abnormal cells, such as cancer cells.

By pharmaceutically acceptable formulation is meant, a composition or formulation that allows for the effective distribution of the nucleic acid molecules of the instant invention in the physical location most suitable for their desired activity. Non-limiting examples of agents suitable for formulation with the nucleic acid molecules of the instant invention include: PEG conjugated nucleic acids, phospholipid conjugated nucleic acids, nucleic acids containing lipophilic moieties, phosphorothioates, P-glycoprotein inhibitors (such as Pluronic P85) which can enhance entry of drugs into various tissues, for example the CNS (Jolliet-Riant and Tillement, 1999, Fundam. Clin. Pharmacol., 13, 16-26); biodegradable polymers, such as poly (DL-lactide-coglycolide) microspheres for sustained release delivery after implantation (Emerich, DF et al, 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999). Other non-limiting examples of delivery strategies, including CNS delivery of the nucleic acid molecules of the instant invention include material described in Boado et al., 1998, J. Pharm. Sci., 87, 1308-1315; Tyler et al., 1999, FEBS Lett., 421, 280-284; Pardridge et al., 1995, PNAS USA., 92, 5592-5596; Boado, 1995, Adv. Drug Delivery Rev., 15, 73-107; Aldrian-Herrada et al., 1998, Nucleic Acids Res., 26, 4910-4916; and Tyler et al., 1999, PNAS USA., 96, 7053-7058. All these references are hereby incorporated herein by reference.

The invention also features the use of the composition comprising surface-modified liposomes containing poly (ethylene glycol) lipids (PEG-modified, or long-circulating liposomes or stealth liposomes). Nucleic acid molecules of the invention can also comprise covalently attached PEG molecules of various molecular weights. These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such liposomes have been shown to accumulate selectively in tumors, presumably by extravasation and capture in the neovascularized target tissues (Lasic et al., Science 1995, 267, 1275-1276; Oku et al.,1995, Biochim. Biophys. Acta, 1238, 86-90). The long-circulating liposomes enhance the pharmacokinetics and pharmacodynamics of DNA and RNA, particularly compared to conventional cationic liposomes which are known to accumulate in tissues of the MPS (Liu et al. J. Biol. Chem. 1995, 42, 24864-24870; Choi et al., International PCT Publication No. WO 96/10391; Ansell et al., International PCT Publication No. WO 96/10390; Holland et al., International PCT Publication No. WO 96/10392; all of which are incorporated by reference herein). Long-circulating liposomes are also likely to protect drugs from nuclease degradation to a greater extent compared to cationic liposomes, based on their ability to avoid accumulation in metabolically aggressive MPS tissues such as the liver and spleen. All of these references are incorporated by reference herein.

The present invention also includes compositions prepared for storage or administration which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985) hereby incorporated by reference herein. For example, preservatives, stabilizers, dyes and flavoring agents can be provided. These include sodium benzoate, sorbic acid and esters of p-hydroxybenzoic acid. In addition, antioxidants and suspending agents can be used.

A pharmaceutically effective dose is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors which those skilled in the medical arts will recognize. Generally, an amount between 0.1 mg/kg and 100 mg/kg body weight/day of active ingredients is administered dependent upon potency of the negatively charged polymer.

The nucleic acid molecules of the invention and formulations thereof can be administered orally, topically, parenterally, by inhalation or spray or rectally in dosage unit formulations containing conventional non-toxic pharmaceutically acceptable carriers, adjuvants and vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a nucleic acid molecule of the invention and a pharmaceutically acceptable carrier. One or more nucleic acid molecules of the invention can be present in association with one or more non-toxic pharmaceutically acceptable carriers and/or diluents and/or adjuvants, and if desired other active ingredients. The pharmaceutical compositions containing nucleic acid molecules of the invention can be in a form suitable for oral use, for example, as tablets, troches, lozenges, aqueous or oily suspensions, dispersible powders or granules, emulsion, hard or soft capsules, or syrups or elixirs.

Compositions intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations. Tablets contain the active ingredient in admixture with non-toxic pharmaceutically acceptable excipients that are suitable for the manufacture of tablets. These excipients can be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monosterate or glyceryl distearate can be employed.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions of the invention can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

Syrups and elixirs can be formulated with sweetening agents, for example glycerol, propylene glycol, sorbitol, glucose or sucrose. Such formulations can also contain a demulcent, a preservative and flavoring and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose any bland fixed oil can be employed including synthetic mono-or diglycerides. In addition, fatty acids such as oleic acid find use in the preparation of injectables.

The nucleic acid molecules of the invention can also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Nucleic acid molecules of the invention can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Dosage levels of the order of from about 0.1 mg to about 140 mg per kilogram of body weight per day are useful in the treatment of the above-indicated conditions (about 0.5 mg to about 7 g per patient per day). The amount of active ingredient that can be combined with the carrier materials to produce a single dosage form varies depending upon the host treated and the particular mode of administration. Dosage unit forms generally contain between from about 1 mg to about 500 mg of an active ingredient.

It is understood that the specific dose level for any particular patient depends upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water.

The nucleic acid molecules of the present invention can also be administered to a patient in combination with other therapeutic compounds to increase the overall therapeutic effect. The use of multiple compounds to treat an indication can increase the beneficial effects while reducing the presence of side effects.

Alternatively, certain of the nucleic acid molecules of the instant invention can be expressed within cells from eukaryotic promoters (e.g., Izant and Weintraub, 1985, Science, 229, 345; McGarry and Lindquist, 1986, Proc. Natl. Acad. Sci., USA 83, 399; Scanlon et al., 1991, Proc. Natl. Acad. Sci. USA, 88, 10591-5; Kashani-Sabet et al., 1992, Antisense Res. Dev., 2, 3-15; Dropulic et al., 1992, J. Virol., 66, 1432-41; Weerasinghe et al., 1991, J. Virol., 65, 5531-4; Ojwang et al., 1992, Proc. Natl. Acad. Sci. USA, 89, 10802-6; Chen et al., 1992, Nucleic Acids Res., 20, 4581-9; Sarver et al., 1990 Science, 247, 1222-1225; Thompson et al., 1995, Nucleic Acids Res., 23, 2259; Good et al., 1997, Gene Therapy, 4, 45; Skillern et al., International PCT Publication No. WO 00/22113; Conrad, International PCT Publication No. WO 00/22114; and Conrad, U.S. Pat. No. 6,054,299; all of these references are hereby incorporated in their totalities by reference herein). Those skilled in the art realize that any nucleic acid can be expressed in eukaryotic cells from the appropriate DNA/RNA vector. The activity of such nucleic acids can be augmented by their release from the primary transcript by a enzymatic nucleic acid (Draper et al., PCT WO 93/23569, and Sullivan et al., PCT WO 94/02595; Ohkawa et al., 1992, Nucleic Acids Symp. Ser., 27, 15-6; Taira et al., 1991, Nucleic Acids Res., 19, 5125-30; Ventura et al., 1993, Nucleic Acids Res., 21, 3249-55; Chowrira et al., 1994, J. Biol. Chem., 269, 25856; all of these references are hereby incorporated in their totalities by reference herein). Gene therapy approaches specific to the CNS are described by Blesch et al., 2000, Drug News Perspect., 13, 269-280; Peterson et al., 2000, Cent. Nerv. Syst. Dis., 485-508; Peel and Klein, 2000, J. Neurosci. Methods, 98, 95-104; Hagihara et al., 2000, Gene Ther., 7, 759-763; and Herrlinger et al., 2000, Methods Mol. Med., 35, 287-312. AAV-mediated delivery of nucleic acid to cells of the nervous system is further described by Kaplitt et al., U.S. Pat. No. 6,180,613.

In another aspect of the invention, nucleic acid molecules of the present invention are preferably expressed from transcription units (see for example Couture et al., 1996, TIG., 12, 510, Skillern et al., International PCT Publication No. WO 00/22113, Conrad, International PCT Publication No. WO 00/22114, and Conrad, U.S. Pat. No. 6,054,299) inserted into DNA or RNA vectors. The recombinant vectors are preferably DNA plasmids or viral vectors. Ribozyme expressing viral vectors can be constructed based on, but not limited to, adeno-associated virus, retrovirus, adenovirus, or alphavirus. Preferably, the recombinant vectors capable of expressing the nucleic acid molecules are delivered as described above, and persist in target cells. Alternatively, viral vectors can be used that provide for transient expression of nucleic acid molecules. Such vectors can be repeatedly administered as necessary. Once expressed, the nucleic acid molecule binds to the target mRNA. Delivery of nucleic acid molecule expressing vectors can be systemic, such as by intravenous or intramuscular administration, by administration to target cells ex-planted from the patient followed by reintroduction into the patient, or by any other means that would allow for introduction into the desired target cell (for a review see Couture et al., 1996, TIG., 12, 510).

In one aspect the invention features an expression vector comprising a nucleic acid sequence encoding at least one of the nucleic acid molecules of the instant invention is disclosed. The nucleic acid sequence encoding the nucleic acid molecule of the instant invention is operable linked in a manner which allows expression of that nucleic acid molecule.

In another aspect the invention features an expression vector comprising: a) a transcription initiation region (e.g., eukaryotic pol I, II or III initiation region); b) a transcription termination region (e.g., eukaryotic pol I, II or III termination region); c) a nucleic acid sequence encoding at least one of the nucleic acid catalyst of the instant invention; and wherein said sequence is operably linked to said initiation region and said termination region, in a manner which allows expression and/or delivery of said nucleic acid molecule. The vector can optionally include an open reading frame (ORF) for a protein operably linked on the 5′ side or the 3′-side of the sequence encoding the nucleic acid catalyst of the invention; and/or an intron (intervening sequences).

Transcription of the nucleic acid molecule sequences are driven from a promoter for eukaryotic RNA polymerase I (pol I), RNA polymerase II (pol II), or RNA polymerase III (pol III). Transcripts from pol II or pol III promoters are expressed at high levels in all cells; the levels of a given pol II promoter in a given cell type depends on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990, Proc. Natl. Acad. Sci. U S A, 87, 6743-7; Gao and Huang 1993, Nucleic Acids Res., 21, 2867-72; Lieber et al., 1993, Methods Enzymol., 217, 47-66; Zhou et al., 1990, Mol. Cell. Biol., 10, 4529-37). All of these references are incorporated by reference herein. Several investigators have demonstrated that nucleic acid molecules, such as ribozymes expressed from such promoters can function in mammalian cells (e.g. Kashani-Sabet et al., 1992, Antisense Res. Dev., 2, 3-15; Ojwang et al., 1992, Proc. Natl. Acad. Sci. USA, 89, 10802-6; Chen et al., 1992, Nucleic Acids Res., 20, 4581-9; Yu et al., 1993, Proc. Natl. Acad. Sci. USA, 90, 6340-4; L'Huillier et al., 1992, EMBO J, 11, 4411-8; Lisziewicz et al., 1993, Proc. Natl. Acad. Sci. U.S.A, 90, 8000-4; Thompson et al., 1995, Nucleic Acids Res., 23, 2259; Sullenger & Cech, 1993, Science, 262, 1566; all of these references are incorporated by reference herein). More specifically, transcription units such as the ones derived from genes encoding U6 small nuclear (snRNA), transfer RNA (tRNA) and adenovirus VA RNA are useful in generating high concentrations of desired RNA molecules such as ribozymes in cells (Thompson et al., supra; Couture and Stinchcomb, 1996, supra; Noonberg et al., 1994, Nucleic Acid Res., 22, 2830; Noonberg et al., U.S. Pat. No. 5,624,803; Good et al., 1997, Gene Ther., 4, 45; Beigelman et al., International PCT Publication No. WO 96/18736; all of these publications are incorporated by reference herein. The above ribozyme transcription units can be incorporated into a variety of vectors for introduction into mammalian cells, including but not restricted to, plasmid DNA vectors, viral DNA vectors (such as adenovirus or adeno-associated virus vectors), or viral RNA vectors (such as retroviral or alphavirus vectors) (for a review see Couture and Stinchcomb, 1996, supra).

In another aspect the invention features an expression vector comprising nucleic acid sequence encoding at least one of the nucleic acid molecules of the invention, in a manner which allows expression of that nucleic acid molecule. The expression vector comprises in one embodiment; a) a transcription initiation region; b) a transcription termination region; c) a nucleic acid sequence encoding at least one said nucleic acid molecule; and wherein said sequence is operably linked to said initiation region and said termination region, in a manner which allows expression and/or delivery of said nucleic acid molecule.

In another embodiment the expression vector comprises: a) a transcription initiation region; b) a transcription termination region; c) an open reading frame; d) a nucleic acid sequence encoding at least one said nucleic acid molecule, wherein said sequence is operably linked to the 3′-end of said open reading frame; and wherein said sequence is operably linked to said initiation region, said open reading frame and said termination region, in a manner which allows expression and/or delivery of said nucleic acid molecule. In yet another embodiment the expression vector comprises: a) a transcription initiation region; b) a transcription termination region; c) an intron; d) a nucleic acid sequence encoding at least one said nucleic acid molecule; and wherein said sequence is operably linked to said initiation region, said intron and said termination region, in a manner which allows expression and/or delivery of said nucleic acid molecule.

In another embodiment, the expression vector comprises: a) a transcription initiation region; b) a transcription termination region; c) an intron; d) an open reading frame; e) a nucleic acid sequence encoding at least one said nucleic acid molecule, wherein said sequence is operably linked to the 3′-end of said open reading frame; and wherein said sequence is operably linked to said initiation region, said intron, said open reading frame and said termination region, in a manner which allows expression and/or delivery of said nucleic acid molecule.

### EXAMPLE

The following are non-limiting examples showing techniques useful in isolating nucleic acid molecules of the instant invention.

### Example 1

### Diagnostic Screen

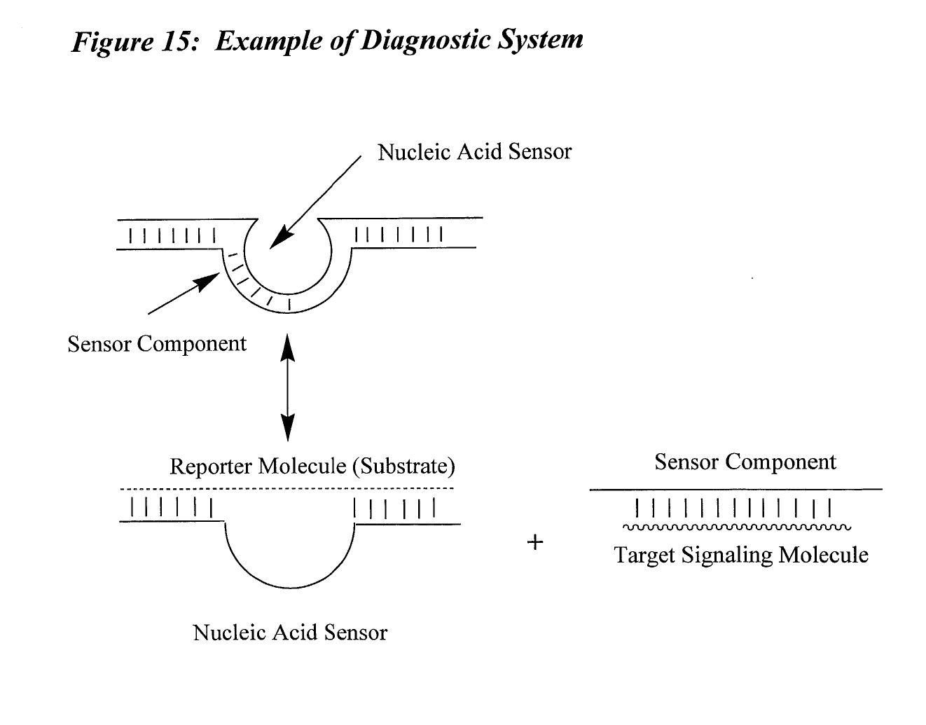

A series of sensor molecules with trans-acting sensor component sequences were designed. Table III shows the sequences that were used in this test. Sequences with names beginning with R- were the reporter sequences used in this experiment, and those were beginning with SM- were nucleic acid sensor molecules. Sequences beginning with S- were sensor component sequences that were designed to bind to portions of the sensor molecule sequences (to varying degrees) and to prevent the sensor molecule from binding and cleaving reporter molecules; these sequences are shown in lower case because they were synthesized using 2′-O-methyl nucleotides in order to increase binding affinity. The one sequence labeled T-2a represents the target signaling molecule sequence which was designed to bind to the sensor component sequences so as to prevent them from inhibiting the sensor molecule activity. The system construct is shown in FIG. 15.

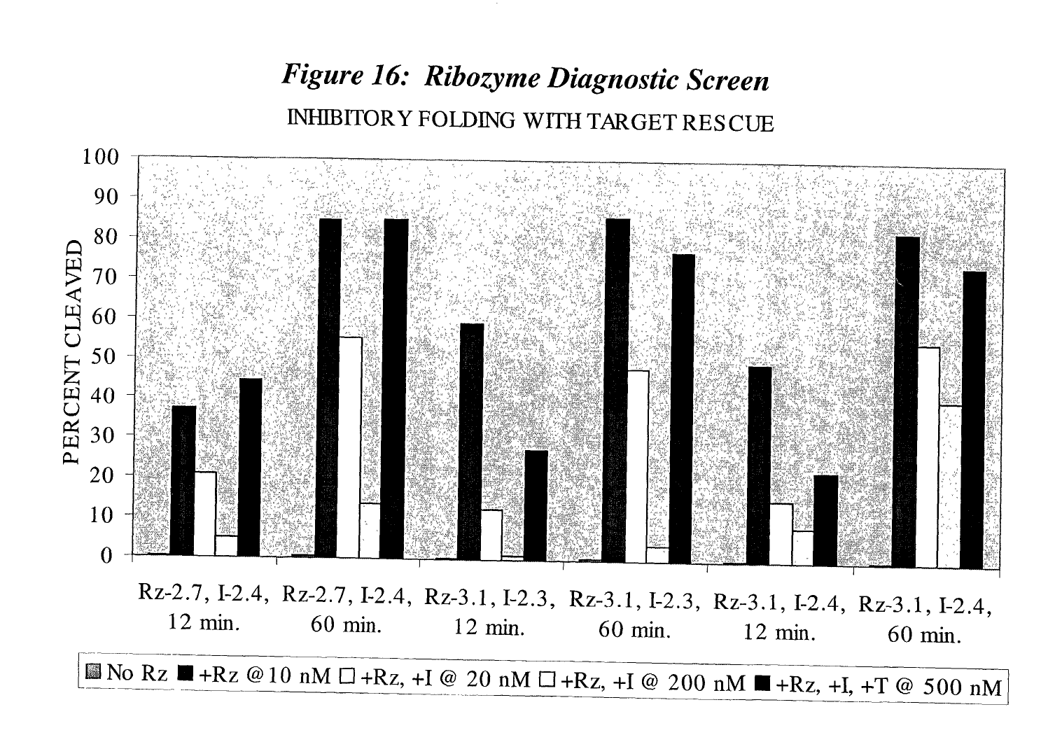

FIG. 16 shows the results of testing some of these sensor molecule/sensor component combinations in a cleavage assay. The reporter molecules were 5′-end labeled with ³²P-phosphate and incubated for 12 or 60 minutes in either: (1) buffer alone (50 mM Tris, pH 7.5, 10 mM MgCl₂), or in the presence of (2) 10 nM sensor molecule, (3) 10 nM sensor molecule plus 20 nM sensor component, (4) 10 nM sensor molecule plus 200 nM sensor component, or (5) 10 nM sensor molecule plus 20 nM sensor component and 500 nM target signaling molecule. At the end of the incubation the reactions were loaded onto a PAGE gel to separate cleaved reporter from uncleaved reporter. The gel was imaged on a Molecular Dynamics phosphorimager and quantitated to determine the percent of reporter molecule cleaved under each set of conditions. Control reactions were carried out to ensure that addition of sensor component or target signaling sequence, without sensor molecule, did not result in reporter cleavage; only 0.2-0.4% of reporter was cleaved under these conditions.

FIG. 16 shows that sensor molecule alone results in 40-60% cleavage of the reporter molecule after 1 minute, and 85% cleavage after 60 minutes for three sensor molecules. When 20 nM sensor component is added to the reaction, the cleavage activity is reduced by 30-70%. When 200 nM sensor component is added, the cleavage activity is reduced by 50-99%. Finally, addition of 500 nM target signaling molecule to a reaction containing 10 nM sensor molecule and 20 nM target signaling molecule results in almost complete recovery of the cleavage activity up to the level observed with sensor molecule alone.

### Example 2

Auto-ligating Nucleic Acid Molecules

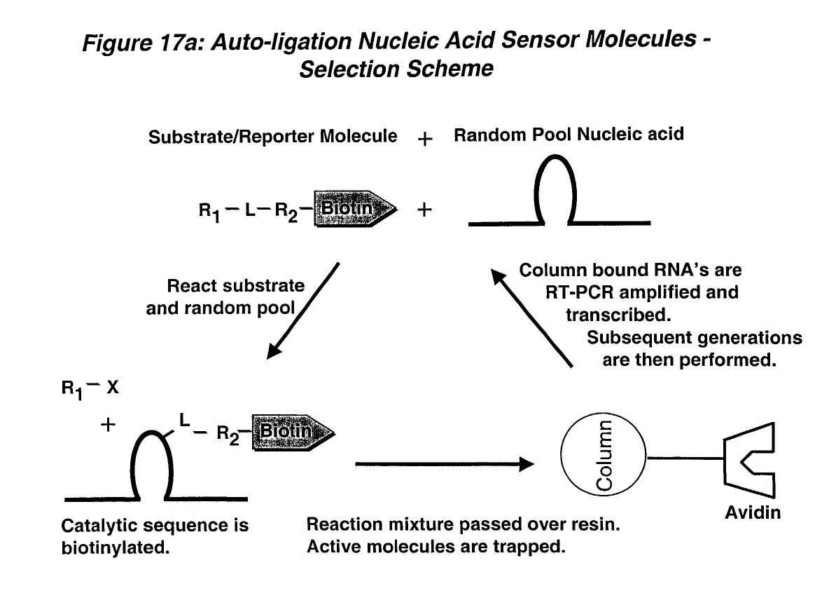

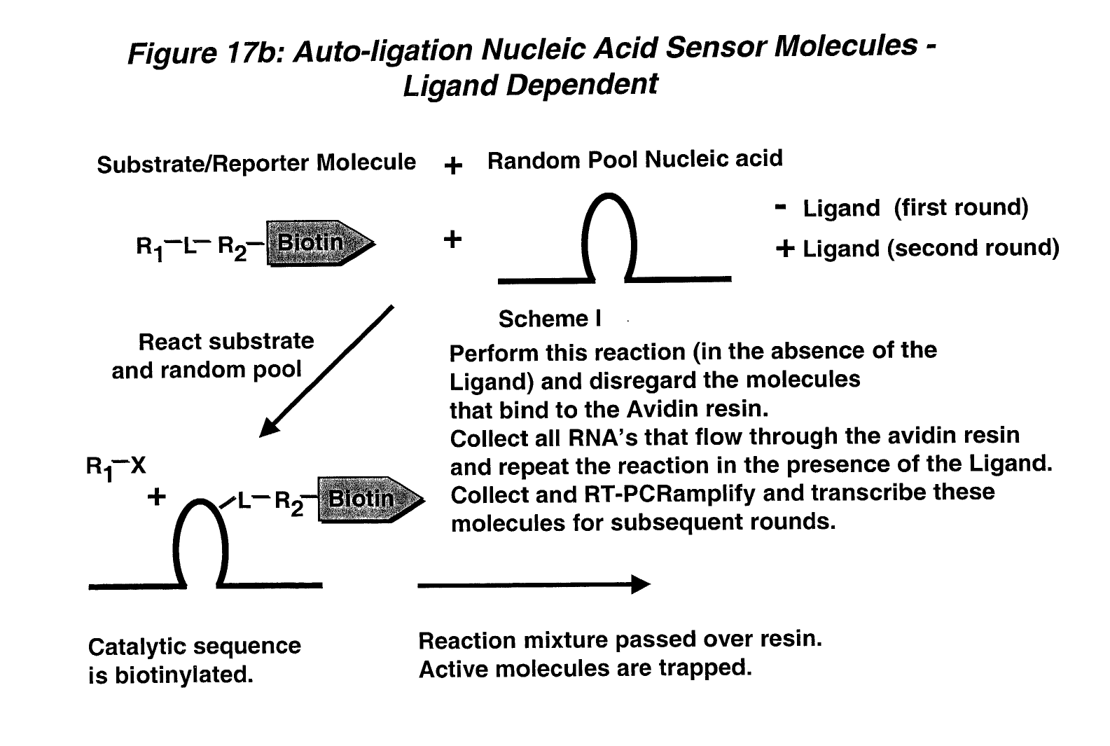

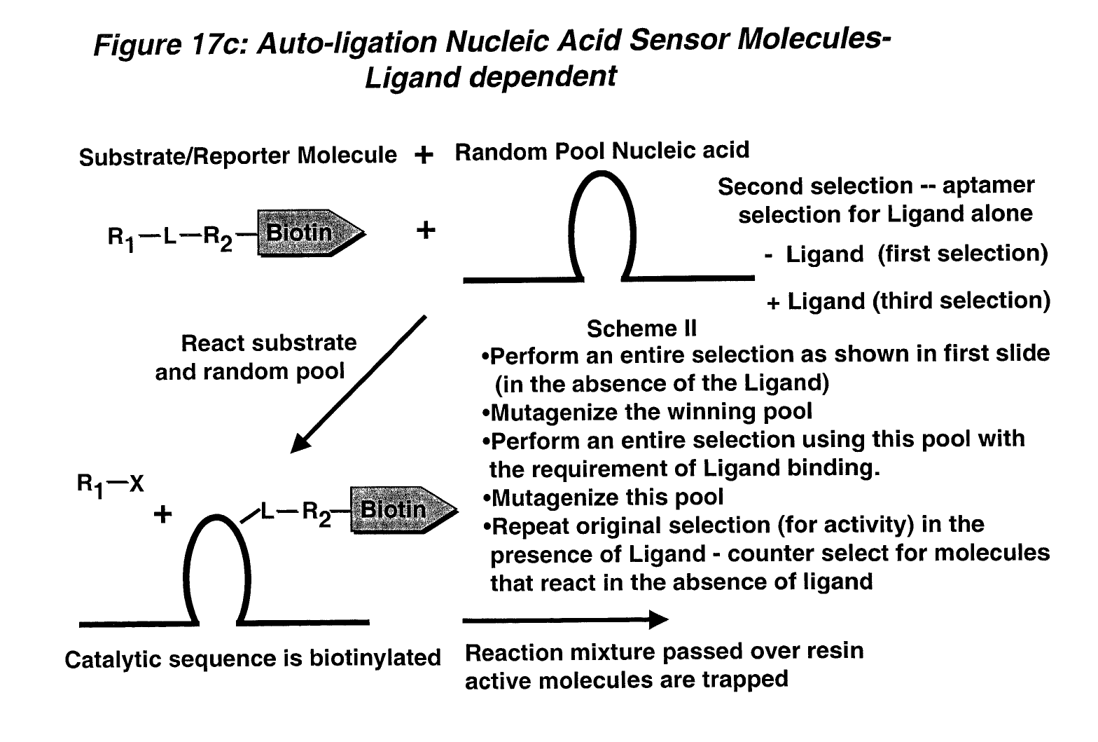

FIG. 17 is a schematic representation of the method of the invention used to isolate nucleic acid molecules capable of auto-ligation reactions useful, for example, in diagnostic applications. FIG. 17a shows the general selection scheme used for isolating active sequences. A random pool of nucleic acid, such as RNA is combined with a substrate molecule comprising the structure R1—O—R2-Biotin, wherein R1 is selected from the group consisting of methyl, hydrogen, phosphate, nucleoside, nucleotide, oligonucleotide, R2 is selected from the group comprising molecules capable of generating a detectable signal, such as molecular beacons, small molecules, fluorophores, chemophores, ionophores, radio-isotopes, photophores, peptides, proteins, enzymes, antibodies, nucleic acids, and enzymatic nucleic acids, L represents a linker which can be present or absent, and “—” represents a covalent bond. Catalytically active sequences are biotinylated. The reaction mixture is passed over a solid support derivatized with Avidin, resulting in the capture of the biotinylated, catalytically active sequence pool. The support bound sequences are amplified by methods known in the art. FIG. 17b shows the selection of the initial pool of sequences that provide ligation activity, and subsequent selection of molecules that are active in the presence of a ligand. Initially, selection of catalytic sequences takes place in the absence of the ligand. The active molecules isolated from the first round of selection that initially bind to the Avidin derivatized support are eliminated. Molecules that pass through the support are re-selected in the presence of the ligand. The re-selected pool that binds to the support after reaction in the presence of the ligand is amplified by methods known in the art and transcribed for subsequent rounds of selection. FIG. 17c shows another selection strategy for isolating nucleic acid molecules capable of autoligation in the presence of a ligand. In this case, an initial selection takes place in the absence of the ligand to select sequences with autoligation activity. This pool is mutagenized by methods known in the art. The resulting mutagenized pool is selected for ligand binding activity by methods known in the art, for example, by using ligand affinity chromatography or gel shift assays. The resulting pool is mutagenized by methods known in the art. The original selection (for activity) is repeated in the presence of the ligand of diagnostic interest, with counterselection for molecules that react in the absence of the ligand.

### Example 3

### Isomerase Nucleic Acid Molecules

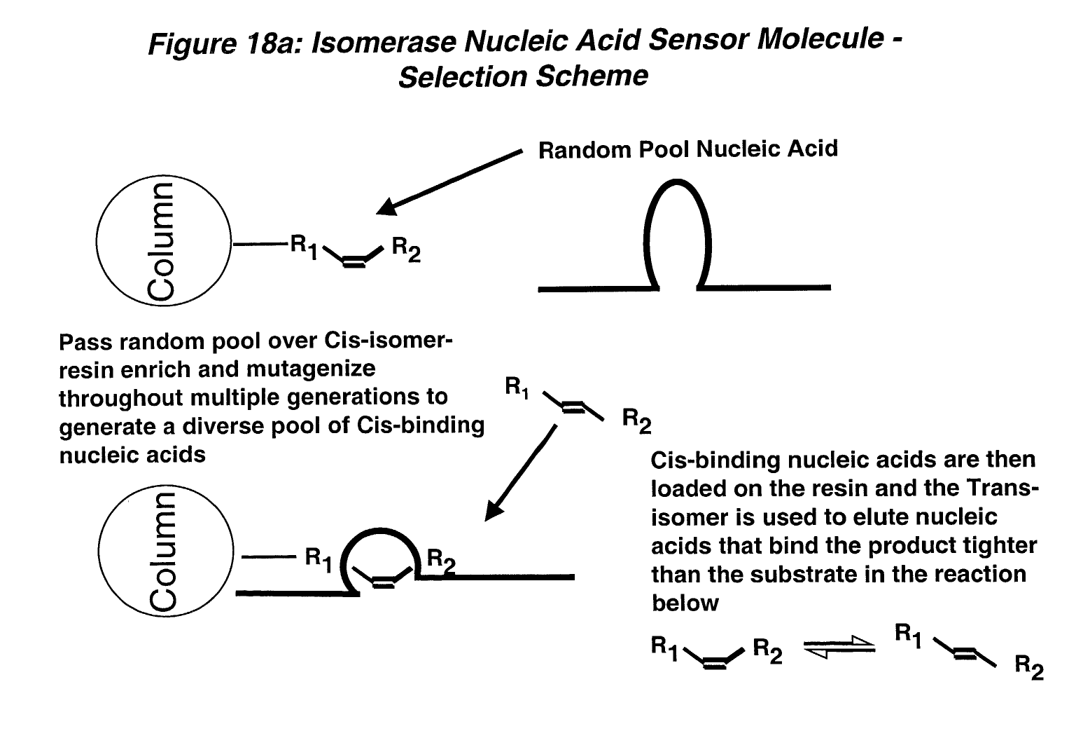

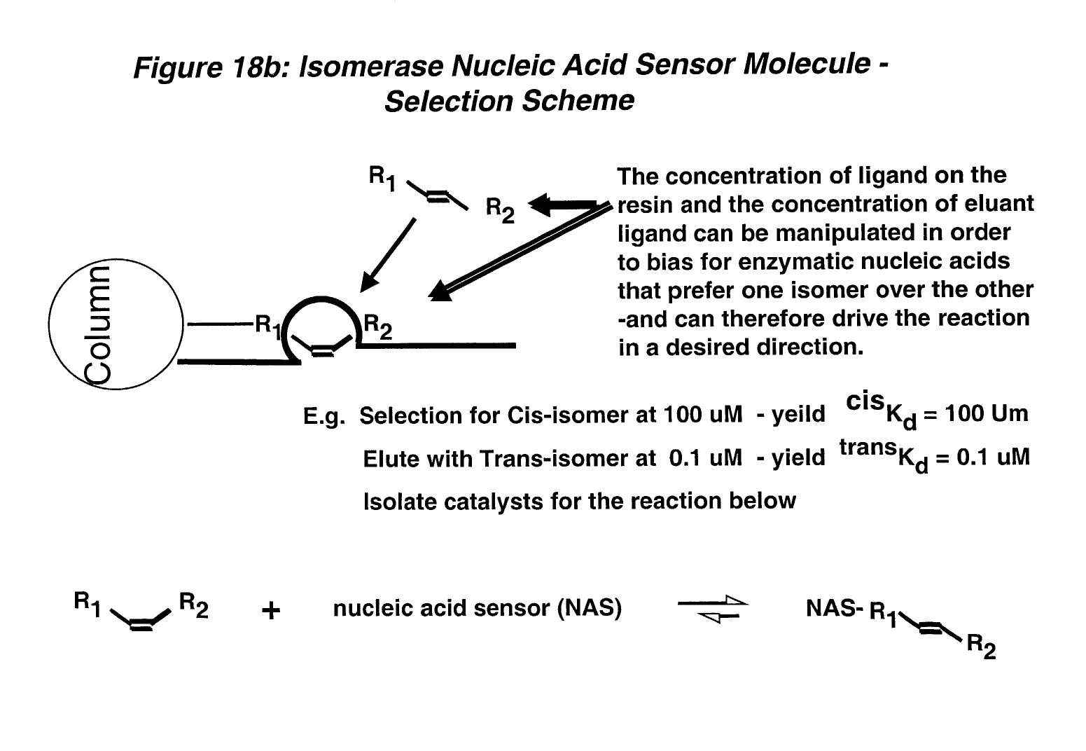

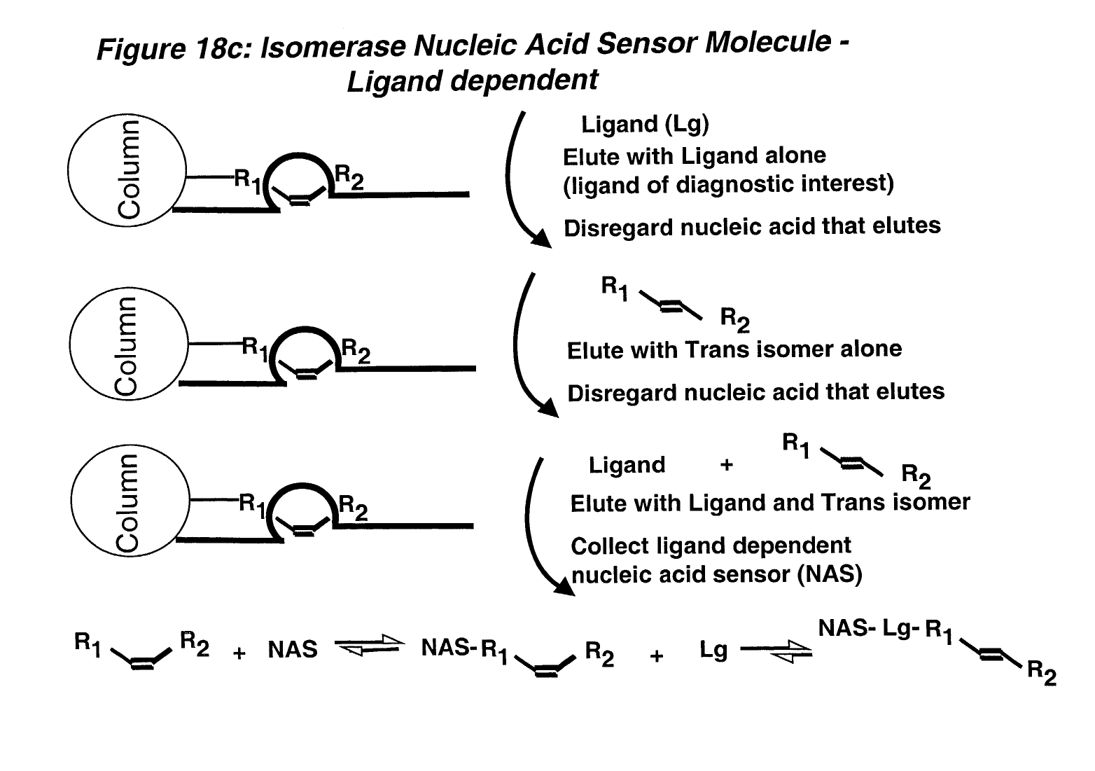

FIG. 18 is a schematic representation of the method of the invention used to isolate nucleic acid sensor molecules capable of catalyzing isomerization reactions useful, for example in diagnostic applications. R1 and R2 represent compounds, which can be the same or different, capable of generating a detectable signal or quenching a detectable signal when an isomerization event takes place, comprising molecular beacons, small molecules, fluorophores, chemophores, ionophores, radio-isotopes, photophores, peptides, proteins, enzymes, antibodies, nucleic acids, and enzymatic nucleic acids, L represents a linker which can be present or absent, and “—” represents a covalent bond. FIG. 18a shows the general selection scheme used for isolating active sequences. A random pool of nucleic acid sequences are passed over the complex of interest, derivatized to a solid support. The representative example of the complex shown in the figure consists of two fluorescent molecules joined together via a cis-carbon double bond linkage. Alternatively, a trans-carbon double bond linkage can be used. The selection pool is enriched and mutagenized throughout multiple generations to generate a diverse pool of “cis” binding sequences. Cis-binding nucleic acid molecules are then loaded onto the resin and the corresponding trans isomer of the complex is used to elute sequences that bind the trans-isomer tighter than the cis-isomer. FIG. 18b shows how the concentration of cis-isomer on the resin and the concentration of trans-isomer eluant can be manipulated in order to select sequences that prefer binding to one isomer over the other, and can therefor drive the reaction in the desired direction. FIG. 18c shows a selection scheme for isolating ligand dependent nucleic acid isomerase molecules from the initial selection pool from FIG. 18a . A counter-selection takes place in which sequences that are bound to the cis-isomer complex are eluted with the ligand of diagnostic interest. An additional counter-selection takes place in which sequences that are bound to the cis-isomer complex are eluted with the ligand of diagnostic interest. A selection then takes place in which sequences remaining from the counter-selection rounds that are bound to the cis-isomer complex are eluted with a mixture of the ligand of diagnostic interest and the trans-isomer complex, the eluted ligand dependent nucleic acid catalyst sequences are amplified and transcribed by methods known in the art.

### Example 4

### Detection of HCV RNA

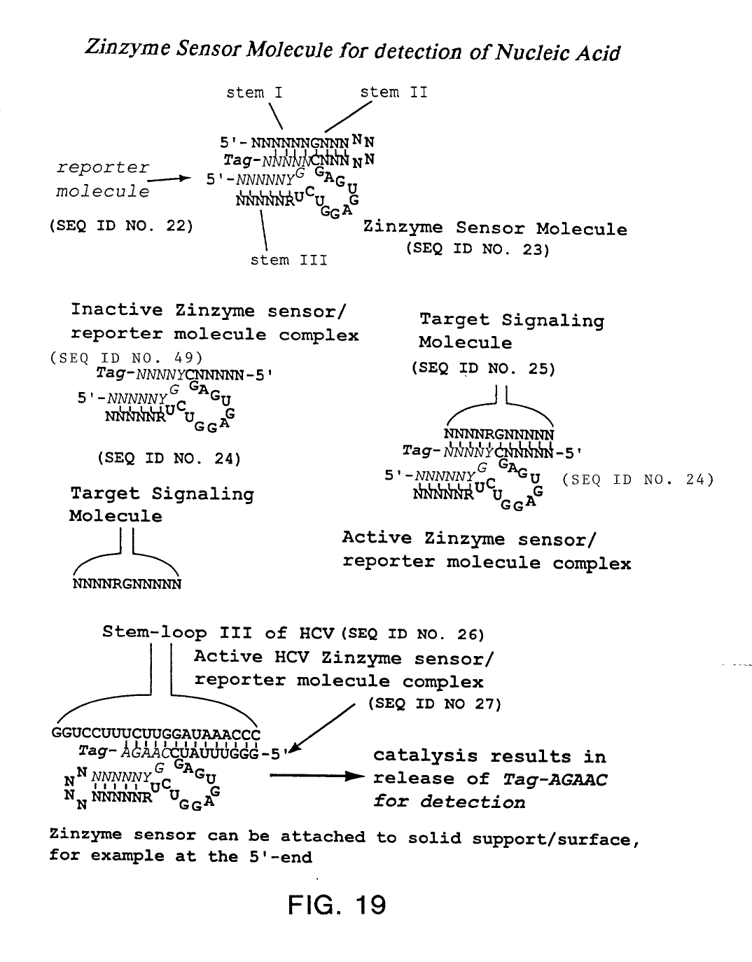

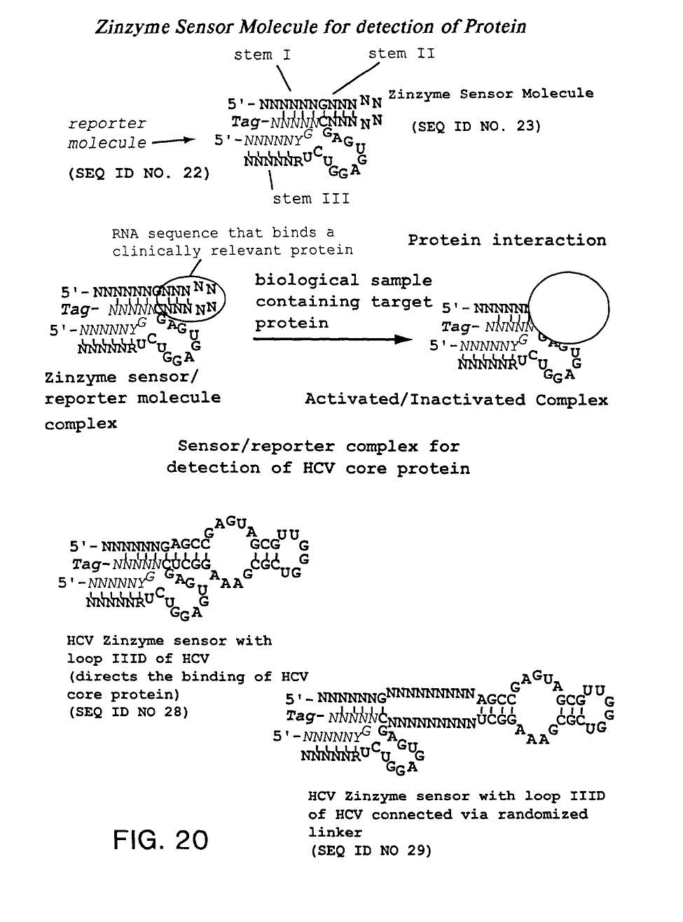

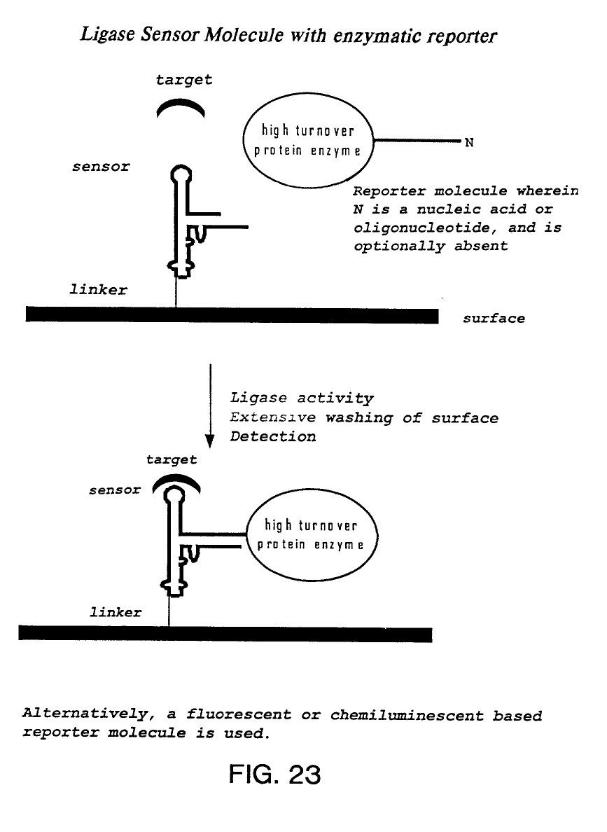

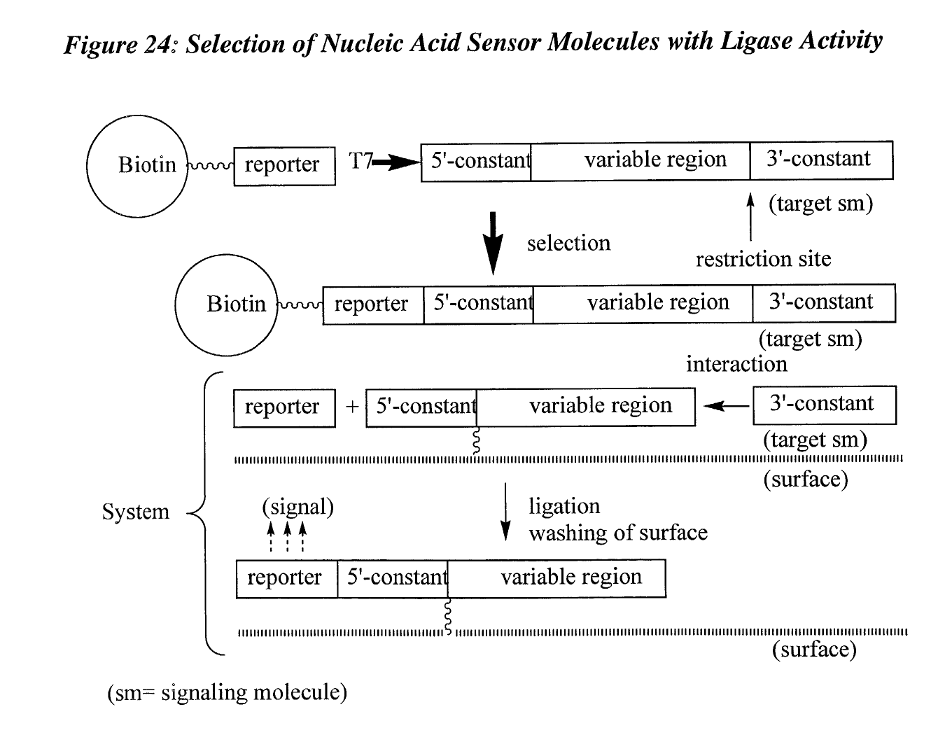

A nucleic acid sensor molecule of the instant invention can be utilized to detect the presence of hepatitis C virus (HCV) in a sample of human blood. A system comprising a human blood sample, a reporter molecule such as a high turnover enzyme, and a nucleic acid sensor molecule attached to a solid support surface is used. The nucleic acid sensor molecule comprises an enzymatic nucleic acid component including an HCV specific sensor component, wherein in response to an interaction of HCV RNA or HCV core proteins with the nucleic acid sensor molecule, the enzymatic nucleic acid component catalyzes a chemical reaction. The reaction can comprise cleavage and release of a reporter molecule when HCV RNA is used as a target signaling agent (see for example FIG. 19), or when HCV core proteins are used as a target signaling agent (see for example FIG. 20). Alternatively, the reaction can comprise the attachment of the reporter molecule to the nucleic acid sensor molecule in the presence of the HCV target (see for example FIG. 23 or FIG. 24). In the case of a sensor molecule that ligates a reporter molecule, the system is subjected to conditions under which free reporter molecules are removed from the system, for example, by washing the surface of the solid support.

The reporter molecule in the system can comprise a conjugated enzyme, such as luciferase, alkaline phosphatase, or horseradish peroxidase. Covalent attachment of the reporter molecule to the nucleic acid sensor molecule takes place in the presence of HCV RNA or core protein. The system is subjected to conditions that cause free reporter molecule to be removed from the system, for example, washing the surface of a solid phase system. A substrate for the conjugated enzyme is contacted with the system under conditions where conversion of the substrate by the immobilized enzyme generates an amplified signal, for example a precipitate, that is detected on the surface of the system (see FIG. 23 or FIG. 24).

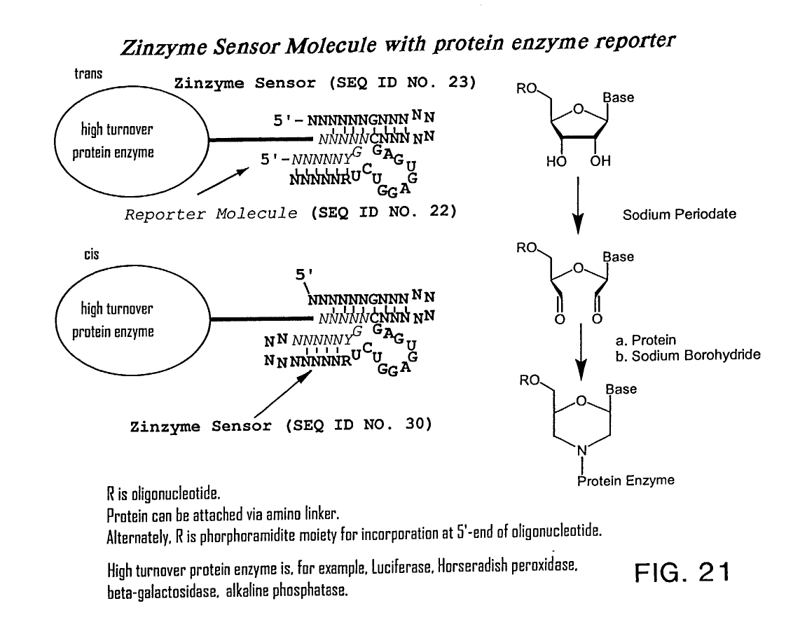

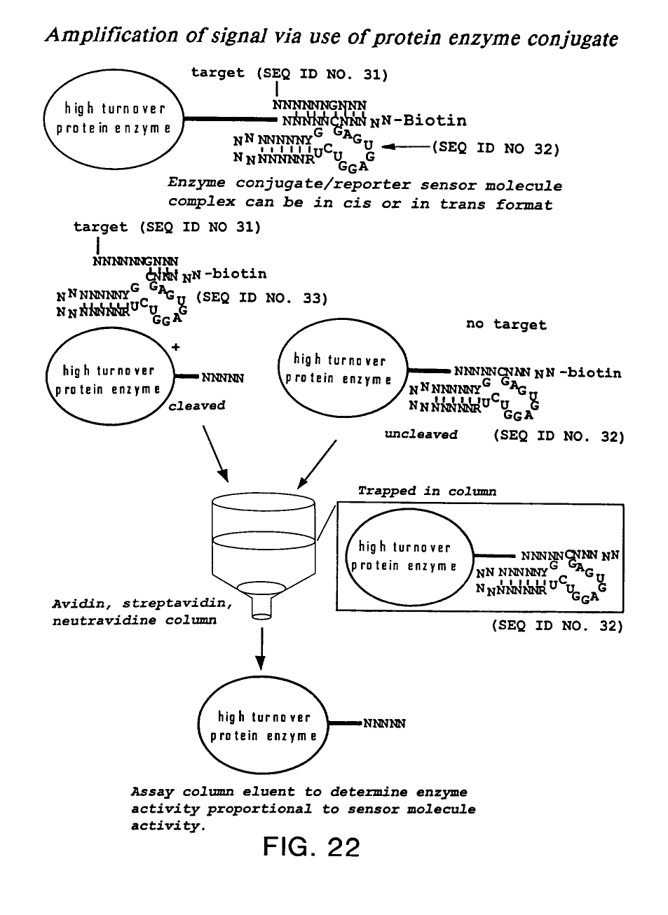

A system in which cleavage of a reporter molecule rather than ligation is used to detect the presence of a target signaling molecule is shown in FIG. 22. An example for the attachment of a reporter enzyme to a nucleic acid sequence is shown in FIG. 21. A system comprising a solution phase and a solid phase is used, wherein a biotin conjugated Zinzyme sensor molecule is used to detect the presence of a target signaling molecule (for example HCV RNA). In the presence of the HCV RNA target signaling molecule (“target” in the figure), the reporter molecule component of the sensor molecule is released from the sensor molecule when the sensor molecule interacts with the target signaling molecule in solution. The solution phase components are passed through a solid phase derivatized with avidin, streptavidin, or neutravidin. The eluent is assayed to indicate the presence of the high turnover enzyme by providing substrate for the enzyme. Enzyme activity is indicative of the presence of the HCV RNA in the system. Alternatively, the sensor molecule is attached to a solid support, for example covalently, wherein a sample is passed through or is passed over the support bound sensor molecule. The eluent is assayed to indicate the presence of the high turnover enzyme by providing substrate for the enzyme. Enzyme activity is indicative of the presence of the HCV RNA in the system.

The use of nucleic acid sensor molecules as described herein is amenable to point of care applications, enabling the simple and efficient detection of analytes in a clinical setting.

### Example 5

### Nucleic Acid Sensor Circuit

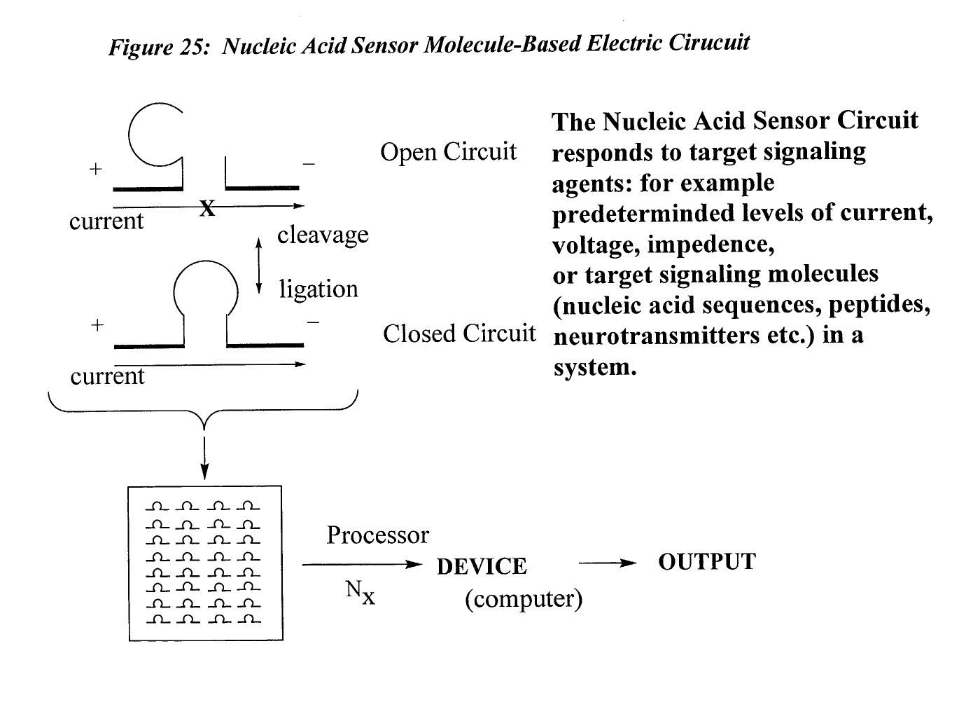

FIG. 25 describes a process whereby a nucleic acid signaling molecule is used in a nucleic acid circuit. The nucleic acid sensor molecule can be used to open or close an electronic circuit. In response to a target signaling agent, for example current, the nucleic acid sensor molecule catalyzes a chemical reaction comprising ligation in response to a predetermined current or cleavage in response to a predetermined current. The nucleic acid circuit is thereby modulated between an open and a closed state based on the predetermined input current that is applied to the circuit. A plurality of such circuits that comprise nucleic acid sensor modulation can be used in a variety of electronic devices, and can substitute solid state or silicon-based circuits in such devices. For example, computer processors comprising a plurality of nucleic acid sensor molecule based-circuits can be used in a computer device. Open and closed nucleic acid sensor molecule based-circuits can be used to generate or respond to binary code, creating a readable output. Processing of nucleic acids by nucleic acid sensor molecules can be used to generate more complex code, for example where particular nucleic acid sequences represent different code variables.

### Example 6

### Target Inhibition of Nucleic Acid Sensor Molecule

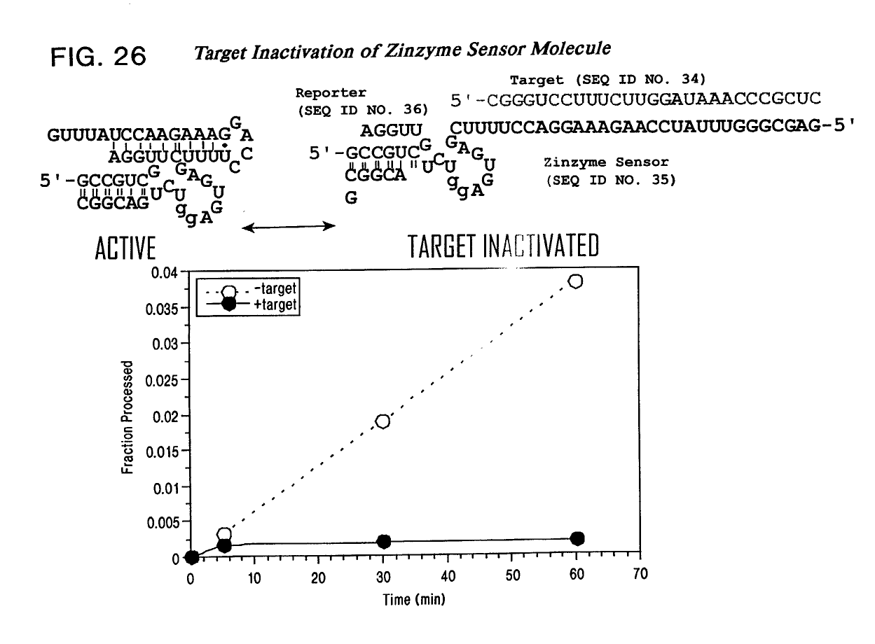

FIG. 26 shows a non-limiting example of target signaling molecule inactivation of a zinzyme sensor molecule. In the absence of the target (SEQ ID NO. 31), the zinzyme sensor molecule (SEQ ID NO. 32) catalyzes the cleavage of a reporter molecule (SEQ ID NO. 33). Reaction conditions: 140 mM KCl10 mM NaCl, 20 mM HEPES pH 7.4, 1mM MgCl2, 1 mM CaCl2, 400 nM Nucleic acid sensor, 400 nM Target, Trace of labeled reporter (˜10 nM), 25 μl reaction volume, Nucleic acid sensor, target and reporter were heated at 75° C. for 3 min, cooled to 37° C. and cleavage initiated by the addition of MgCl2 and CaCl2.

### Example 7

### Target Activation of Nucleic Acid Sensor Molecule

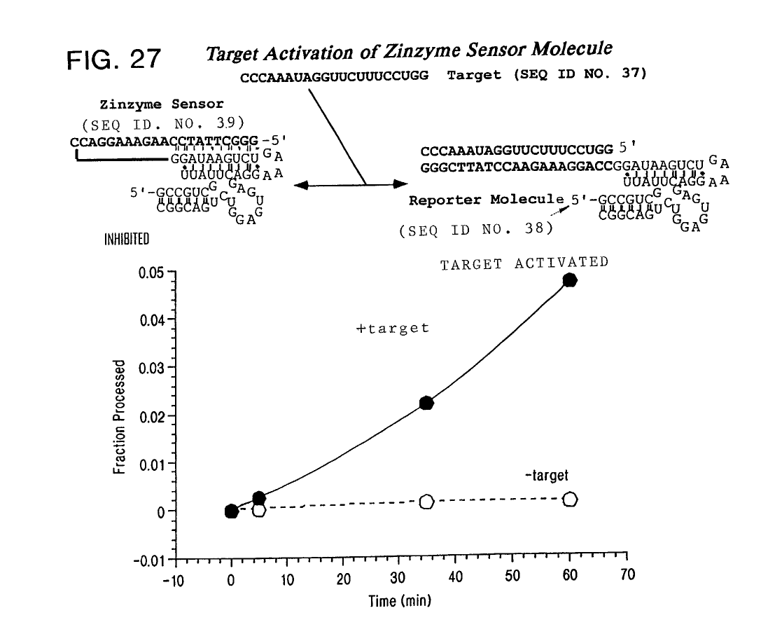

FIG. 27 shows a non-limiting example of target signaling molecule activation of a zinzyme sensor molecule. In the presence of the target (SEQ ID NO. 34), the zinzyme sensor molecule (SEQ ID NO. 35) catalyzes the cleavage of a reporter molecule (SEQ ID NO. 36). Reaction conditions: 140 mM KCl, 10 mM NaCl, 20 mM HEPES pH 7.4, 1 mM MgCl2, 1 mM CaCl2, 400 nM Nucleic acid sensor, 400 nM Target, Trace of labeled reporter (˜10 nM), 25 μl reaction volume, Nucleic acid sensor, target and reporter were heated at 75° C. for 3 min, cooled to 37° C. and cleavage initiated by the addition of MgCl2 and CaCl2.

### Example 8

### Protein (Erk) target Activation of nucleic Acid Sensor Molecule

One method for protein detection contemplated by the invention utilizes a catalytically attenuated enzymatic nucleic acid molecule that is fused to a high affinity RNA ligand for a target protein in such a way that target association induces catalytic activity. A variation of combinatorial selection methods can be easily and quickly used to create high affinity RNA ligands (RNA sensor domains) for specific proteins. Combinatorial selection of RNA aptamers has been automated and multiplexed, providing a high throughput method for their production. Very much like antibodies, RNA aptamers display picomolar affinities for their targets and can discriminate between protein homologs, isoforms, and even different activation states of the same protein. Alternately, RNA sensor domains can be obtained from natural sources, such as the RNA binding domains of a virus (e.g. rev response elements and TAR elements of HIV) or eukaryotic RNA binding proteins (e.g. protein kinase PKR, promoters, RNA polymerase, ribosomal RNA binding domains etc.). In addition, a random sequence can be attached to an attenuated enzymatic nucleic acid molecule and through the use of combinatorial selection, allosteric nucleic acid molecules can be isolated that are modulated in the presence of a target signaling agent or molecule.

This approach relies upon binding of a protein target to an RNA aptamer domain in the nucleic acid sensor molecule to induce catalytic activity. To accomplish this activation, the sensor and enzymatic nucleic acid molecule domains are fused via a third element, a communication module, that is responsible promoting enzymatic nucleic acid molecule catalysis upon target binding. The communication module is a nucleic acid sequence or sequences that promote a conformational rearrangement of the enzymatic nucleic acid molecule domain into its active structure upon target binding. Two routes exist for the production of communication modules: rational design or combinatorial selection. One approach utilizes rational design where pre-made communication module or modules are fused to preexisting enzymatic nucleic acid molecule and aptamer domains in a modular strategy.

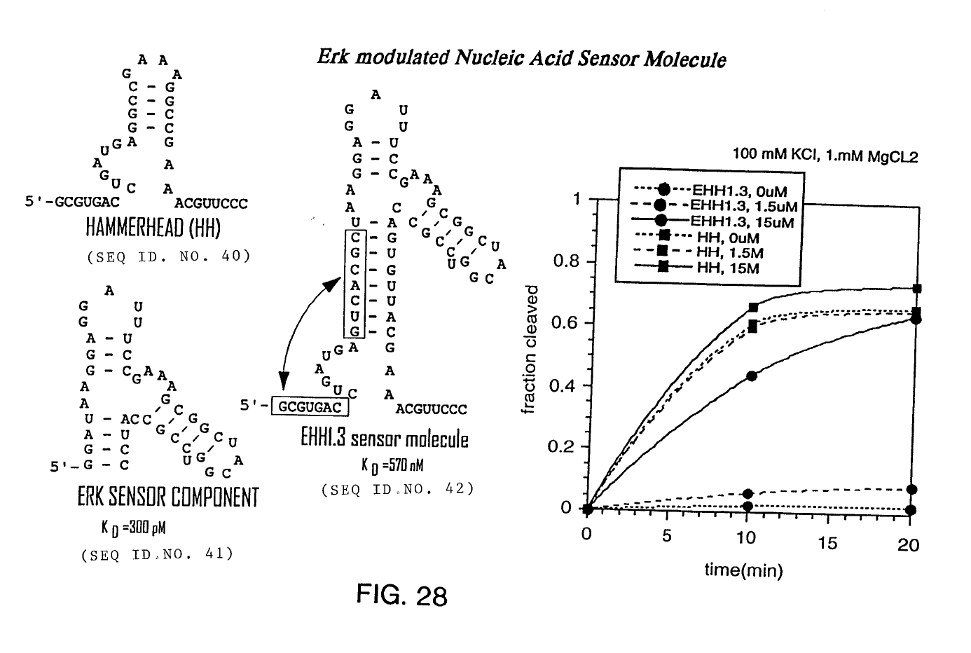

An RNA sensor domain that binds to protein ERK2 (Erk) was appended to a variant of the hammerhead enzymatic nucleic acid molecule through a communication module developed through rational design. The salient feature of this design strategy is that substrate-binding elements in the enzymatic nucleic acid molecule domain are sequestered by complementary allosteric effector sequences present in the communication module in the absence of target. Target association with the sensor domain forces an alternative RNA conformation in which the substrate binding elements become available for interaction with cleavage substrate, thus promoting catalysis. FIG. 28 shows a non-limiting example of a nucleic acid sensor molecule that is modulated by a protein target signaling molecule, Erk. In the presence of the target protein (Erk), the nucleic acid sensor molecule (SEQ ID NO. 39) catalyzes the cleavage of a reporter molecule. Reaction conditions: 100 mM KCl, 1 mM MgCl2, 10 mM Tris 7.5, 10 μM ERK protein, 1 μM HH enzymatic nucleic acid molecule, Vf=19 μl, 34° C. for 30 minutes, trace 5′ labeled substrate (1 μl).

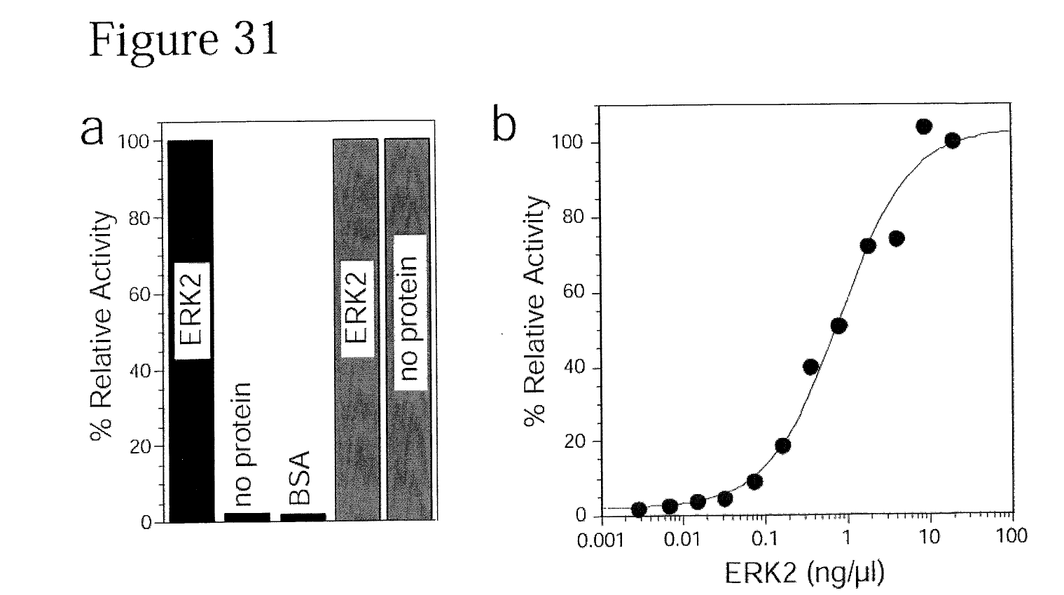

This nucleic acid sensor displays little catalytic activity in the absence of the ERK2 protein but is activated approximately one hundred fold in the presence of recombinant ERK2 (FIG. 31al ). No nucleic acid sensor activation is observed if bovine serum albumin (BSA) replaces ERK2 in the reaction, indicating that activation specifically requires ERK2. An enzymatic nucleic acid molecule that does not contain the ERK2 sensor component displays nearly identical activity in the presence or absence of the protein target (FIG. 31a). To examine the dependence of activation on the concentration of ERK2, various amounts of ERK2 were added to different reactions (FIG. 31b). One half-maximal nucleic acid sensor molecule activation is promoted by ˜800 pg/μl ERK2. Because the parental RNA sensor component displays an affinity of 8 pg/μl for ERK2, the sensitivity of this sensor molecule activation by ERK2 can likely be increased a further hundred fold by combinatorial optimization of the sensor molecule.

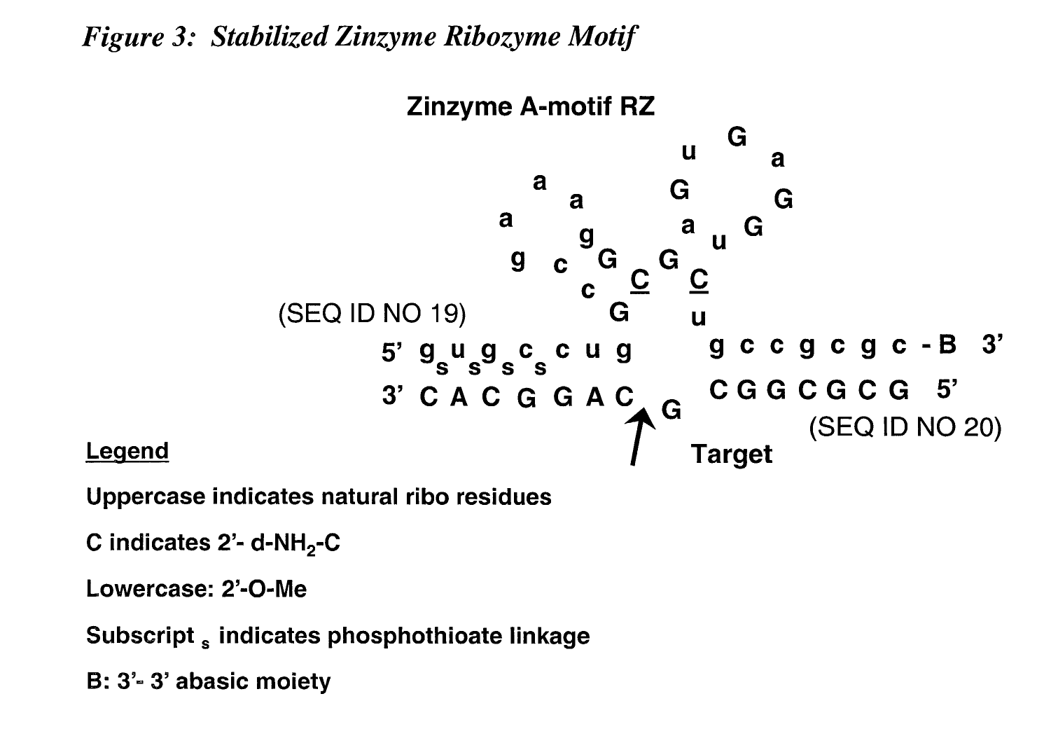

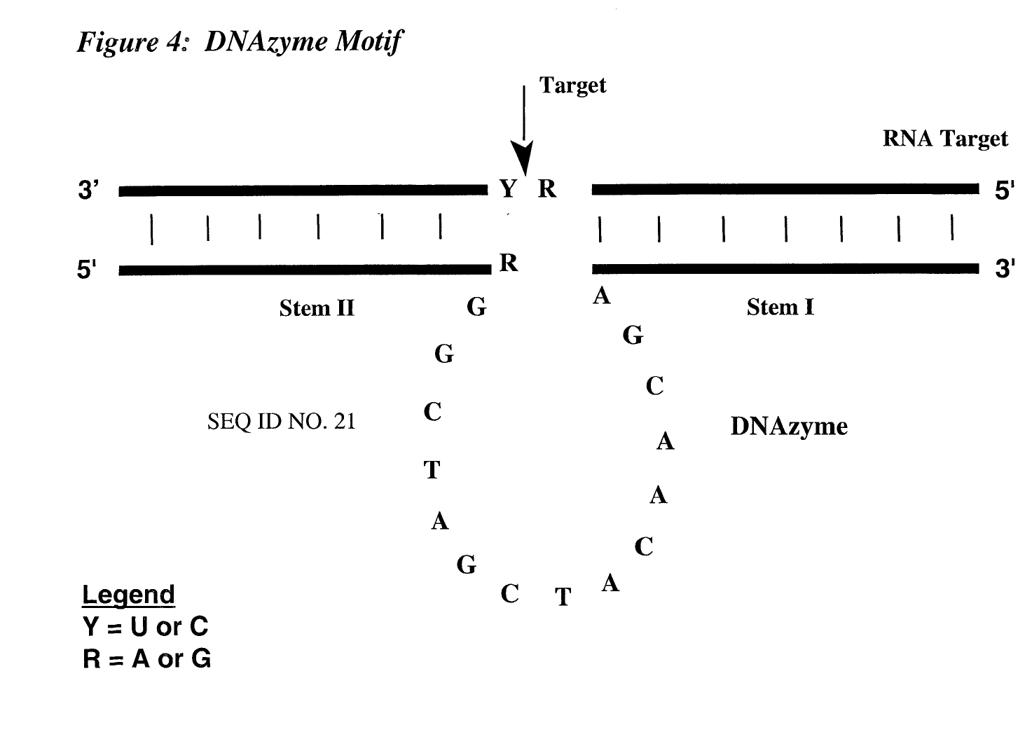

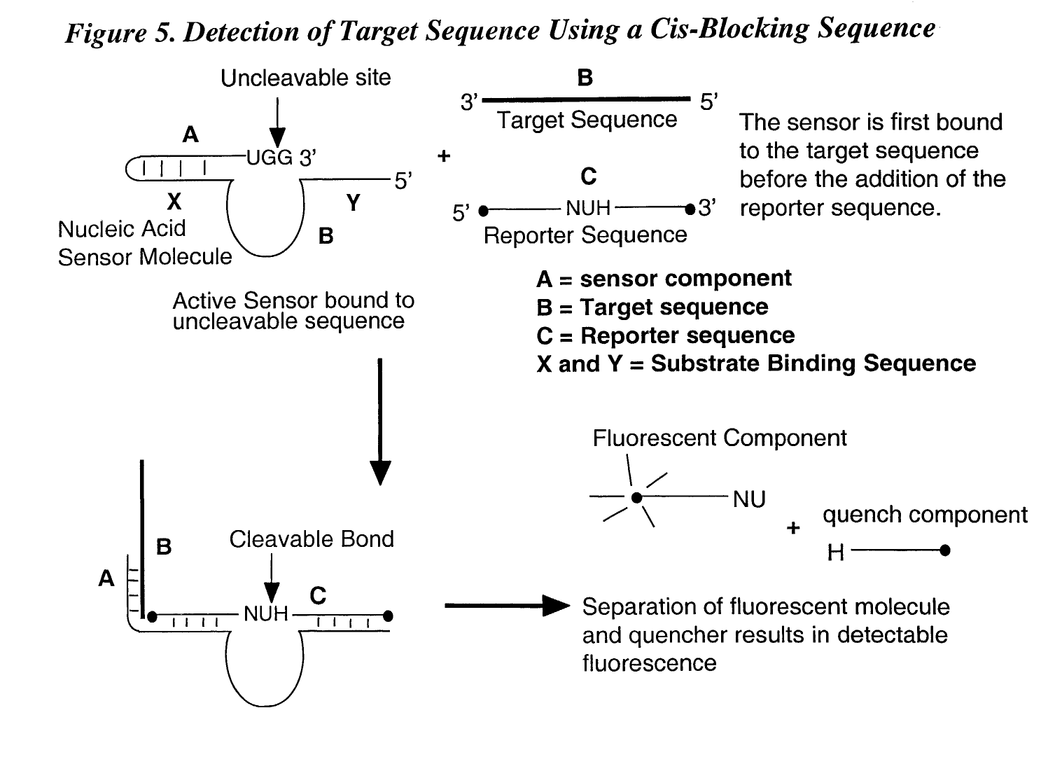

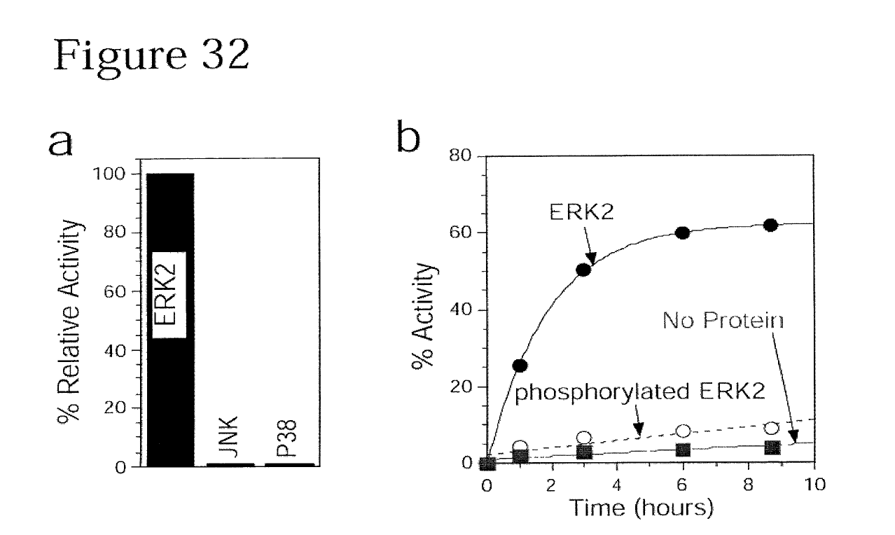

Thus, this technology has a sensitivity comparable to that displayed by standard antibody based ELISA assays. The specificity of allosteric activation also compares favorably with antibody based approaches. ERK2 is a member of the mitogen activated protein kinase (MAPK) family, different members of which are implicated in a wide range of cellular processes including cancer (ERK2) and inflammation and apoptosis (P38 and JNK). These kinases are highly homologous, displaying up to 45% amino acid sequence identity. To examine the specificity of ERK2 responsive nucleic acid sensor molecule (allozyme), applicant attempted to activate the nucleic acid sensor molecule with P38 and JNK. These proteins did not activate the allozyme (FIG. 32a), nor did bovine serum albumin (FIG. 31a). ERK function is up regulated by a specific phosphorylation event that alters its structure. The RNA sensors used for the allozyme described here preferentially associate with the unactivated form of ERK2. Phosphorylated ERK2 was substituted for unactivated ERK2 in an allozyme reaction to assess its ability to activate enzymatic nucleic acid catalysis. Phosphorylated protein fails to activate the allozyme (FIG. 32b). Thus, protein responsive nucleic acid sensor molecules (allozymes) can not only distinguish between different protein homologs, but also between different activation states of the same protein. Another approach used in the design of nucleic acid sensor molecules involves combinatorial selection of nucleic acid sensor molecules that are capable of catalysis in the presence of a predetermined target. For example, the evolution of protein binding nucleic acid sensor molecules to a protein, such as ERK2, can take place with modification of a known enzymatic nucleic acid motif. A variable region is introduced into the sequence and selective pressure is applied in iterative rounds of isolation and amplification, for example the isolation and amplification of sequences that cleave a substrate in cis in the presence of the target molecule. In a non-limiting example, such a random region is introduced into the Zinzyme stem-loop region (5′-CCGAAAGG-3′ ) shown in FIG. 3.

### Example 9

### Half-zinzyme Nucleic Acid sensor Molecule (Halfzyme)

Applicant has developed a generalizable methodology for the production of nucleic acid sensor molecules that are activated by target nucleic acids. This technology is based on enzymatic nucleic acids that, in the absence of a target nucleic acid, are catalytically inactive because they lack portions of the catalytic core and substrate recognition elements. In this ‘half-ribozyme’ or ‘halfzyme’ system, catalysis can occur if a specific target nucleic acid supplies the sequences required for catalysis in trans.

Although many enzymatic nucleic acid motifs can be used for the halfzyme strategy, one system uses the Zinzyme motif (FIG. 3) in which the substrate nucleic acid is attached to the enzymatic nucleic acid. This motif is small (about 32 nucleotides), carries modifications that confer a half-life in serum of greater than 100 hours, and has minimal target sequence requirements (5′-N3-RG-N3-3′, where N=any nucleotide and R=A or G). Thus, this motif is readily synthesized, has the ability to detect different sequences, and can be used directly in serum or other biological fluids.

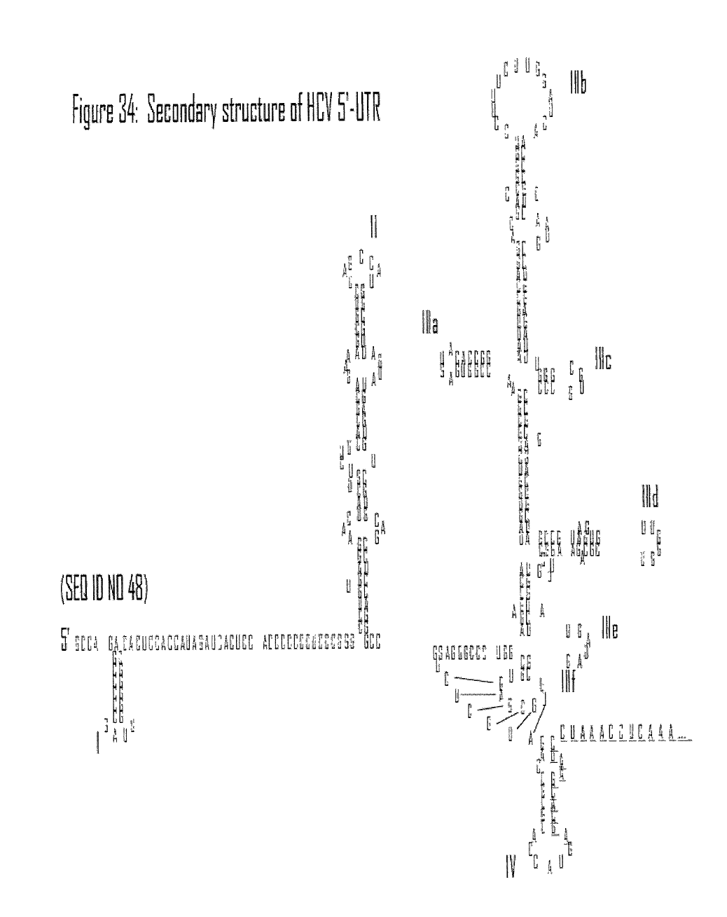

Applicant has tested the feasibility of the halfzyme approach using the Zinzyme motif and the Hepatitis C Virus genome as a model target. A synthetic oligoribonucleotide representing loop IIIB of the 5′ untranslated region (UTR), a universally conserved region of the HCV genome, activates catalysis of a rationally designed, sequence matched halfzyme. In the absence of oligoribonucleotide target no nucleic acid sensor molecule activity is detected. Other regions of the HCV 5′-UTR (see FIG. 34) can be similarly used in the design of other halfzymes contemplated by the invention.

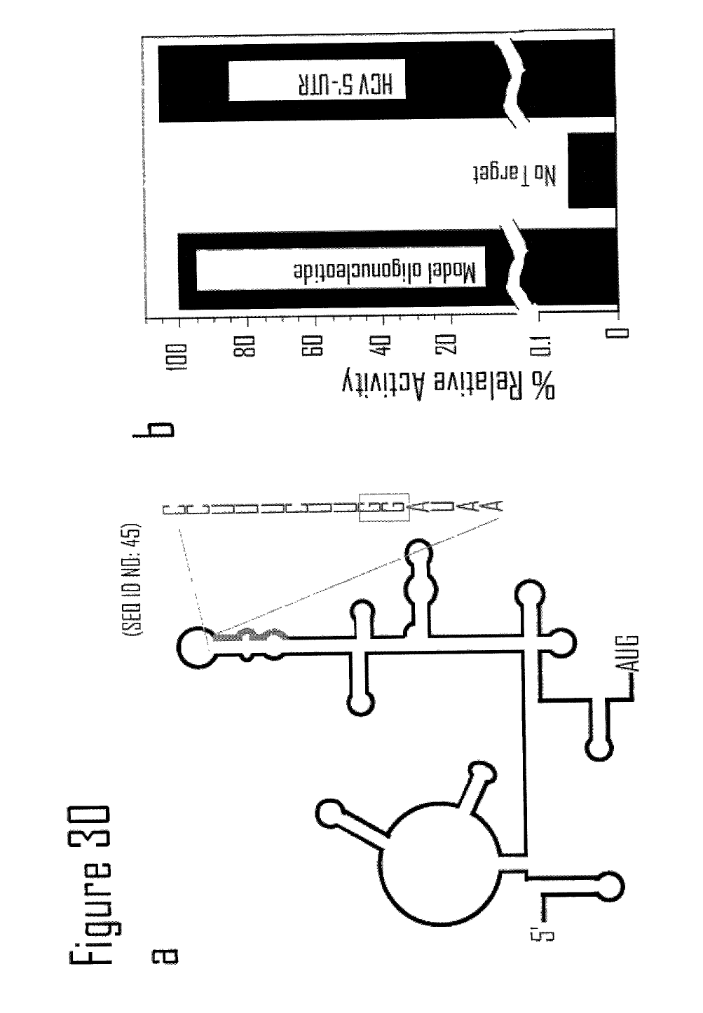

In this example, the halfzyme is activated by a target sequence derived from intact HCV genome. The 5′-UTR of HCV folds into a compact three-dimensional structure independent of the remaining portion of the HCV genome. To disrupt this structure so that UTR-derived loop IIIB sequences are accessible for activation of the halfzyme, a simple 20 minute pre-treatment step was inserted into the assay. Pre-treatment of the HCV 5′-UTR with a DNA oligonucleotide complementary to stem III and RNase H (FIG. 30a) is sufficient to activate halfzyme catalysis to the same extent as that observed with a short synthetic oligoribonucleotide (FIG. 30b). Thus, the halfzyme used in these studies can efficiently detect the presence of a conserved sequence element derived from the HCV genome. Target capture by a halfzyme is determined by the affinity of the halfzyme for its target and can be described in molarity by a dissociation constant. The value of this dissociation constant can be rationally engineered into the halfzyme, allowing 100% target capture when halfzyme used in the assay is in excess of this concentration.

A primary concern of any technology aimed at detecting low concentrations of nucleic acids is its sensitivity. The halfzyme approach is unique because catalysis is only promoted in the presence of a sequence-matched target and because 100% target capture can be achieved by manipulating halfzyme concentration. Therefore, single molecule detection is theoretically possible by this approach provided that an adequate signal amplification system is in place. Given the enormous flexibility of possible signal amplification and detection systems accommodated by the technology, signal detection should not define the limit of sensitivity of this technology. In practice, the limit of sensitivity of this approach is dictated by the uncatalyzed rate of substrate cleavage promoted under the assay conditions used. Therefore, the salient issue in terms of sensitivity becomes the relative rate of catalyzed versus uncatalyzed substrate cleavage. A virtue of the system is that both the assay conditions and halfzyme activity can be manipulated to maximize this rate differential.

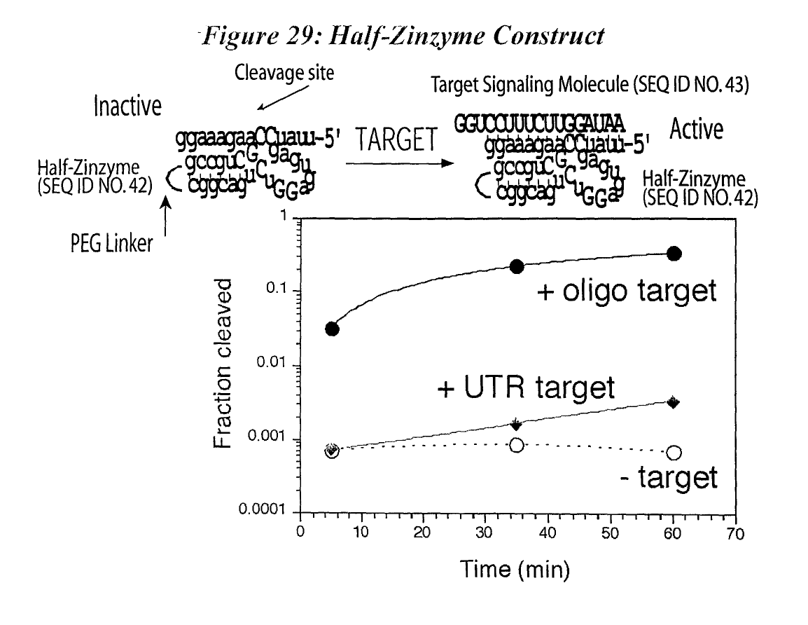

FIG. 29 shows a non-limiting example of a “half-zinzyme” nucleic acid sensor molecule with a PEG linker that is modulated by the 5′-UTR of the Hepatitis C virus (HCV 5′-UTR). The figure shows both inactive and active forms of the zinzyme sensor molecule (SEQ ID NO. 42). In the presence of the target signaling oligonucleotide (SEQ ID NO. 43) which represents the stem loop IIIB of the HCV 5′-UTR, the zinzyme sensor demonstrates an activity increase of three logs in cleaving the reporter molecule component of the sensor molecule as shown in the graph (+oligo target) as compared to the sensor molecule in the absence of the target. In the presence of the full length 350 nt. HCV 5′-UTR, the zinzyme sensor molecule demonstrates an almost one log increase in activity in cleaving the reporter molecule component of the sensor molecule. Reaction conditions: 140 mM KCl, 10 mM NaCl, 20 mM HEPES pH 7.4, 1 mM MgCl2, 1 mM CaCl2, 400 nM Nucleic acid sensor, 400 nM Target, Trace of labeled reporter (˜10 nM), 25 μl reaction volume, Nucleic acid sensor, target and reporter were heated at 75° C. for 3 min, cooled to 37° C. and cleavage initiated by the addition of MgCl2 and CaCl2.

### Example 10

### Nucleic Acid Sensor Ligase

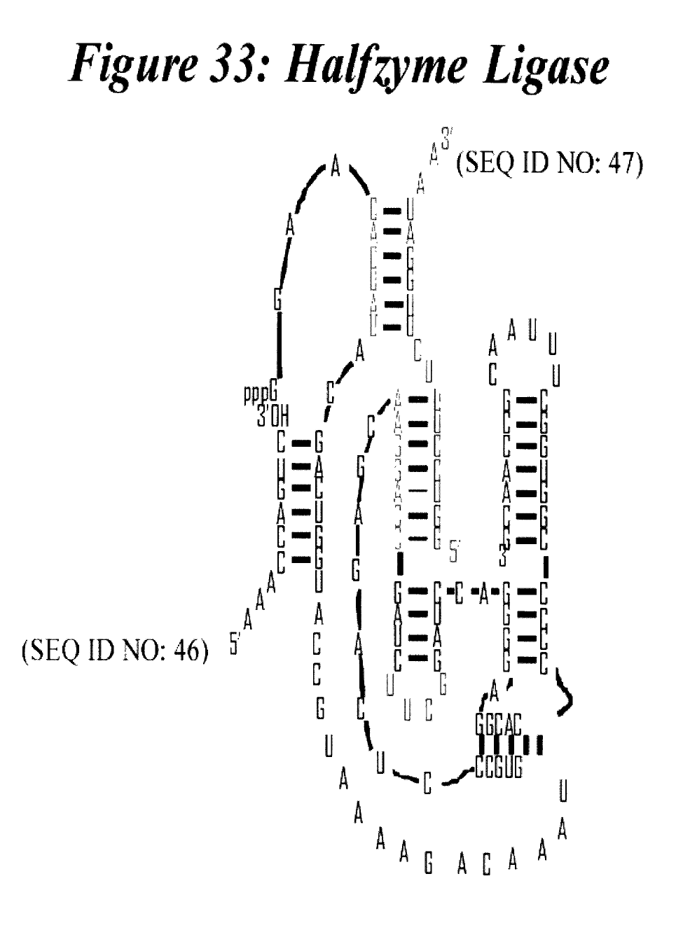

A ligase derived from the Bartel class I ligase (Ekland et al., 1995, Science, 269, 364-370) was prepared. Three different constructs carried various 3′ truncations. These segments were supplied in trans as oligonucleotide HCV sequence. One ligase, termed HZBART-2 showed ligation rate 107 fold above background ligation (FIG. 33).

Ligation reactions were performed at room temperature in 30 mM Tris, pH 7.5, 200 mM KCl, 60 mM MgCl2 and 0.6 mM EDTA. Halfzyme ligases (1 μM) with corresponding effector oligonucleotide (1 μM) were heated in water at 90° C. for 2 min and cooled at room temperature for 10 min followed by the addition of salt, buffer and 32P-labeled substrate oligonucleotide (0.1 mM final concentration). Reactions were carried out for 60 min at room temperature and stopped by the addition of 1 volume of gel loading buffer (7M urea, 100 mM EDTA) and snap cooling on ice. Products were separated on 20% denaturing polyacrylamide gel electrophoresis.

### Other Uses

The nucleic acid sensor molecules of this invention can be used as diagnostic tools to examine genetic drift and mutations within diseased cells or to detect the presence of a specific RNA in a cell. The close relationship between nucleic acid sensor molecule activity and the structure of the target RNA allows the detection of mutations in any region of the molecule which alters the base-pairing and three-dimensional structure of the target RNA. By using multiple nucleic acid sensor molecules described in this invention, one can map nucleotide changes which are important to RNA structure and function in vitro, as well as in cells and tissues. Cleavage of target RNAs with nucleic acid sensor molecules can be used to inhibit gene expression and define the role (essentially) of specified gene products in the progression of disease. In this manner, other genetic targets can be defined as important mediators of the disease. These experiments can lead to better treatment of the disease progression by affording the possibility of combinational therapies (e.g., multiple nucleic acid sensor molecules targeted to different genes, nucleic acid target molecules coupled with known small molecule inhibitors, or intermittent treatment with combinations of nucleic acid sensor molecules and/or other chemical or biological molecules). Other in vitro uses of nucleic acid sensor molecules of this invention comprise detection of the presence of mRNAs associated with a disease-related condition. Such RNA is detected by determining the presence of a cleavage product after treatment with an enzymatic nucleic acid molecule using standard methodology.

In a specific example, nucleic acid sensor molecules which cleave only wild-type or mutant forms of the target RNA are used for the assay. The first nucleic acid sensor molecule is used to identify wild-type RNA present in the sample and the second nucleic acid sensor molecule is used to identify mutant RNA in the sample. As reaction controls, synthetic substrates of both wild-type and mutant RNA are cleaved by both nucleic acid sensor molecules to demonstrate the relative nucleic acid sensor molecule efficiencies in the reactions and the absence of cleavage of the “non-targeted” RNA species. The cleavage products from the synthetic substrates also serve to generate size markers for the analysis of wild-type and mutant RNAs in the sample population. Thus, each analysis can require two nucleic acid sensor molecules, two substrates and one unknown sample, which are combined into six reactions. The presence of cleavage products is determined using an RNAse protection assay so that full-length and cleavage fragments of each RNA can be analyzed in one lane of a polyacrylamide gel. It is not absolutely required to quantify the results to gain insight into the expression of mutant RNAs and putative risk of the desired phenotypic changes in target cells. The expression of mRNA whose protein product is implicated in the development of the phenotype is adequate to establish risk. If probes of comparable specific activity are used for both transcripts, then a qualitative comparison of RNA levels is adequate and decrease the cost of the initial diagnosis. Higher mutant form to wild-type ratios are correlated with higher risk whether RNA levels are compared qualitatively or quantitatively.

### Additional Uses

Potential usefulness of sequence-specific nucleic acid sensor molecules of the instant invention have many of the same applications for the study of RNA that DNA restriction endonucleases have for the study of DNA (Nathans et al., 1975 Ann. Rev. Biochem. 44:273). For example, the pattern of restriction fragments can be used to establish sequence relationships between two related RNAs, and large RNAs can be specifically cleaved to fragments of a size more useful for study. The ability to engineer sequence specificity of the enzymatic nucleic acid molecule is ideal for cleavage of RNAs of unknown sequence. Applicant describes the use of nucleic acid molecules to detect gene expression of target genes in bacterial, microbial, fungal, viral, and eukaryotic systems including plant, or mammalian cells.

The nucleic acid sensor molecules of the invention represent a new class of therapeutic agents capable of modulating the expression of target genes, peptides, proteins, and other biologically active molecules in vivo as described herein. The therapeutic activity of nucleic acid sensor molecules of the invention can respond to both internal and external stimuli in a patient, for example the presence of a gene, pathogen, SNP, peptide, protein, RNA, metabolite, neurotransmitter, co-factor, drug, toxin, or physical stimuli such as light, gravity, temperature, and pressure.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The methods and compositions described herein as presently representative of preferred embodiments are exemplary and are not intended as limitations on the scope of the invention. Changes therein and other uses which are encompassed within the spirit of the invention, are defined by the scope of the claims.

It will be readily apparent to one skilled in the art that varying substitutions and modifications can be made to the invention disclosed herein without departing from the scope and spirit of the invention. Thus, such additional embodiments are within the scope of the present invention and the following claims.

The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Other embodiments are within the following claims.

TABLE I
Characteristics of naturally occurring ribozymes

Group I Introns
Size: ˜150 to >1000 nucleotides.
Requires a U in the target sequence immediately 5′ of the cleavage site.
Binds 4-6 nucleotides at the 5′-side of the cleavage site.
Reaction mechanism: attack by the 3′-OH of guanosine to generate
cleavage products with 3′-OH and 5′-guanosine.
Additional protein cofactors required in some cases to help folding and
maintenance of the active structure.
Over 300 known members of this class. Found as an intervening sequence
in Tetrahymena thermophila rRNA, fungal mitochondria, chloroplasts,
phage T4, blue-green algae, and others.
Major structural features largely established through phylogenetic
comparisons, mutagenesis, and biochemical studies [^{i, ii}].
Complete kinetic framework established for one ribozyme [^{iii, iv, v, vi}].
Studies of ribozyme folding and substrate docking underway [^{vii, viii, ix}].
Chemical modification investigation of important residues well
established [^{x, xi}].
The small (4-6 nt) binding site can make this ribozyme too non-specific
for targeted RNA cleavage, however, the Tetrahymena group I intron has
been used to repair a “defective” beta-galactosidase message by
the ligation of new beta-galactosidase sequences onto the defective
message [^{xii}].
RNAse P RNA (M1 RNA)
Size: ˜290 to 400 nucleotides.
RNA portion of a ubiquitous ribonucleoprotein enzyme.
Cleaves tRNA precursors to form mature tRNA [^{xiii}].
Reaction mechanism: possible attack by M²⁺-OH to generate cleavage
products with 3′-OH and 5′-phosphate.
RNAse P is found throughout the prokaryotes and eukaryotes. The RNA
subunit has been sequenced from bacteria, yeast, rodents, and primates.
Recruitment of endogenous RNAse P for therapeutic applications is
possible through hybridization of an External Guide Sequence (EGS) to
the target RNA [^{xiv, xv}].
Important phosphate and 2′ OH contacts recently identified [^{xvi, xvii}]
Group II Introns
Size: >1000 nucleotides.
Trans cleavage of target RNAs recently demonstrated [^{xviii, xix}].
Sequence requirements not fully determined.
Reaction mechanism: 2′-OH of an internal adenosine generates cleavage
products with 3′-OH and a “lariat” RNA containing a
3′-5′ and a 2′-5′ branch point.
Only natural ribozyme with demonstrated participation in DNA
cleavage [^{xx, xxi}] in addition to RNA cleavage and ligation.
Major structural features largely established through phylogenetic
comparisons [^{xxii}].
Important 2′ OH contacts beginning to be identified [^{xxiii}]
Kinetic framework under development [^{xxiv}]
Neurospora VS RNA
Size: ˜144 nucleotides.
Trans cleavage of hairpin target RNAs recently demonstrated [^{xxv}].
Sequence requirements not fully determined.
Reaction mechanism: attack by 2′-OH 5′ to the scissile bond to generate
cleavage products with 2′,3′-cyclic phosphate and 5′-OH ends.
Binding sites and structural requirements not fully determined.
Only 1 known member of this class. Found in Neurospora VS RNA.
Hammerhead Ribozyme
(see text for references)
Size: ˜13 to 40 nucleotides.
Requires the target sequence UH immediately 5′ of the cleavage site.
Binds a variable number nucleotides on both sides of the cleavage site.
Reaction mechanism: attack by 2′-OH 5′ to the scissile bond to generate
cleavage products with 2′,3′-cyclic phosphate and 5′-OH ends.
14 known members of this class. Found in a number of plant pathogens
(virusoids) that use RNA as the infectious agent.
Essential structural features largely defined, including 2 crystal
structures [^{xxvi, xxvii}]
Minimal ligation activity demonstrated (for engineering through in vitro
selection) [^{xxviii}]
Complete kinetic framework established for two or more ribozymes [^{xxix}].
Chemical modification investigation of important residues well
established [^{xxx}].
Hairpin Ribozyme
Size: ˜50 nucleotides.
Requires the target sequence GUC immediately 3′ of the cleavage site.
Binds 4-6 nucleotides at the 5′-side of the cleavage site and a variable
number to the 3′-side of the cleavage site.
Reaction mechanism: attack by 2′-OH 5′ to the scissile bond to generate
cleavage products with 2′,3′-cyclic phosphate and 5′-OH ends.
3 known members of this class. Found in three plant pathogen (satellite
RNAs of the tobacco ringspot virus, arabis mosaic virus and chicory
yellow mottle virus) which uses RNA as the infectious agent.
Essential structural features largely defined [^{xxxi, xxxii, xxxiii, xxxiv}]
Ligation activity (in addition to cleavage activity) makes ribozyme
amenable to engineering through in vitro selection [^{xxxv}]
Complete kinetic framework established for one ribozyme [^{xxxvi}].
Chemical modification investigation of important residues
begun [^{xxxvii, xxxviii}].
Hepatitis Delta Virus (HDV) Ribozyme
Size: ˜60 nucleotides.
Trans cleavage of target RNAs demonstrated [^{xxxix}].
Binding sites and structural requirements not fully determined, although no
sequences 5′ of cleavage site are required. Folded ribozyme contains a
pseudoknot structure [^{xl}].
Reaction mechanism: attack by 2′-OH 5′ to the scissile bond to generate
cleavage products with 2′,3′-cyclic phosphate and 5′-OH ends.
Only 2 known members of this class. Found in human HDV.
Circular form of HDV is active and shows increased nuclease stability [^{xli}]
ⁱ Michel, Francois; Westhof, Eric. Slippery substrates. Nat. Struct. Biol. (1994), 1(1), 5-7.
ⁱⁱ Lisacek, Frederique; Diaz, Yolande; Michel, Francois. Automatic identification of group I intron cores in genomic DNA sequences. J. Mol. Biol. (1994), 235(4), 1206-17.
ⁱⁱⁱ Herschlag, Daniel; Cech, Thomas R.. Catalysis of RNA cleavage by the Tetrahymena thermophila ribozyme. 1. Kinetic description of the reaction of an RNA substrate complementary to the active site. Biochemistry (1990), 29(44), 10159-71.
^{iv} Herschlag, Daniel; Cech, Thomas R.. Catalysis of RNA cleavage by the Tetrahymena thermophila ribozyme. 2. Kinetic description of the reaction of an RNA substrate that forms a mismatch at the active site. Biochemistry (1990), 29(44), 10172-80.
^{v} Knitt, Deborah S.; Herschlag, Daniel. pH Dependencies of the Tetrahymena Ribozyme Reveal an Unconventional Origin of an Apparent pKa. Biochemistry (1996), 35(5), 1560-70.
^{vi} Bevilacqua, Philip C.; Sugimoto, Naoki; Turner, Douglas H.. A mechanistic framework for the second step of splicing catalyzed by the Tetrahymena ribozyme. Biochemistry (1996), 35(2), 648-58.
^{vii} Li, Yi; Bevilacqua, Philip C.; Mathews, David; Turner, Douglas H.. Thermodynamic and activation parameters for binding of a pyrene-labeled substrate by the Tetrahymena ribozyme: docking is not diffusion-controlled and is driven by a favorable entropy change. Biochemistry (1995), 34(44), 14394-9.
^{viii} Banerjee, Aloke Raj; Turner, Douglas H.. The time dependence of chemical modification reveals slow steps in the folding of a group I ribozyme. Biochemistry (1995), 34(19), 6504-12.
^{ix} Zarrinkar, Patrick P.; Williamson, James R.. The P9.1-P9.2 peripheral extension helps guide folding of the Tetrahymena ribozyme. Nucleic Acids Res. (1996), 24(5), 854-8.
^{x} Strobel, Scott A.; Cech, Thomas R.. Minor groove recognition of the conserved G.cntdot.U pair at the Tetrahymena ribozyme reaction site. Science (Washington, D.C.) (1995), 267(5198), 675-9.
^{xi} Strobel, Scott A.; Cech, Thomas R.. Exocyclic Amine of the Conserved G.cntdot.U Pair at the Cleavage Site of the Tetrahymena Ribozyme Contributes to 5′-Splice Site Selection and Transition State Stabilization. Biochemistry (1996), 35(4), 1201-11.
^{xii} Sullenger, Bruce A.; Cech, Thomas R.. Ribozyme-mediated repair of defective mRNA by targeted trans-splicing. Nature (London) (1994), 371 (6498), 619-22.
^{xiii} Robertson, H. D.; Altman, S.; Smith, J. D. J. Biol. Chem., 247, 5243-5251 (1972).
^{xiv} Forster, Anthony C.; Altman, Sidney. External guide sequences for an RNA enzyme. Science (Washington, D.C., 1883-) (1990), 249(4970), 783-6.
^{xv} Yuan, Y.; Hwang, E. S.; Altman, S. Targeted cleavage of mRNA by human RNase P. Proc. Natl. Acad. Sci, USA (1992) 89, 8006-10.
^{xvi} Harris, Michael E.; Pace, Norman R.. Identification of phosphates involved in catalysis by the ribozyme RNase P RNA. RNA (1995), 1(2), 210-18.
^{xvii} Pan, Tao; Loria, Andrew; Zhong, Kun. Probing of tertiary interactions in RNA: 2′- hydroxyl-base contacts between the RNase P RNA and pre-tRNA. Proc. Natl. Acad. Sci. U.S.A. (1995), 92(26), 12510-14.
^{xviii} Pyle, Anna Marie; Green, Justin B.. Building a Kinetic Framework for Group II Intron Ribozyme Activity: Quantitation of Interdomain Binding and Reaction Rate. Biochemistry (1994), 33(9), 2716-25.
^{xix} Michels, William J. Jr.; Pyle, Anna Marie. Conversion of a Group II Intron into a New Multiple-Turnover Ribozyme that Selectively Cleaves Oligonucleotides: Elucidation of Reaction Mechanism and Structure/Function Relationships. Biochemistry (1995), 34(9), 2965-77.
^{xx} Zimmerly, Steven; Guo, Huatao; Eskes, Robert; Yang, Jian; Perlman, Philip S.; Lambowitz, Alan M.. A group II intron RNA is a catalytic component of a DNA endonuclease involved in intron mobility. Cell (Cambridge, Mass.) (1995), 83(4), 529-38.
^{xxi} Griffin, Edmund A., Jr.; Qin, Zhifeng; Michels, Williams J., Jr.; Pyle, Anna Marie. Group II intron ribozymes that cleave DNA and RNA linkages with similar efficiency, and lack contacts with substrate 2′-hydroxyl groups. Chem. Biol. (1995), 2(11), 761-70.
^{xxii} Michel, Francois; Ferat, Jean Luc. Structure and activities of group II introns. Annu. Rev. Biochem. (1995), 64, 435-61.
^{xxiii} Abramovitz, Dana L.; Friedman, Richard A.; Pyle, Anna Marie. Catalytic role of 2′-hydroxyl groups within a group II intron active site. Science (Washington, D.C.) (1996), 271(5254), 1410-13
^{xxiv} Daniels, Danette L.; Michels, William J., Jr.; Pyle, Anna Marie. Two competing pathways for self-splicing by group II introns: a quantitative analysis of in vitro reaction rates and products. J. Mol. Biol. (1996), 256(1), 31-49.
^{xxv} Guo, Hans C. T.; Collins, Richard A.. Efficient trans-cleavage of a stem-loop RNA substrate by a ribozyme derived from Neurospora VS RNA. EMBO J. (1995), 14(2), 368-76.
^{xxvi} Scott, W. G., Finch, J. T., Aaron, K. The crystal structure of an all RNA hammerhead ribozyme: A proposed mechanism for RNA catalytic cleavage. Cell, (1995), 81, 991-1002.
^{xxvii} McKay, Structure and function of the hammerhead ribozyme: an unfinished story. RNA, (1996), 2, 395-403.
^{xxviii} Long, D., Uhlenbeck, O., Hertel, K. Ligation with hammerhead ribozymes. U.S Pat. No. 5,633,133.
^{xxix} Hertel, K. J., Herschlag, D., Uhlenbeck, O. A kinetic and thermodynamic framework for the hammerhead ribozyine reaction. Biochemistry, (1994) 33, 3374-3385. Beigelman, L., et al., Chemical modifications of hammerhead ribozymes. J. Biol. Chem., (1995) 270, 25702-25708.
^{xxx} Beigelman, L., et al., Chemical modifications of hammerhead ribozymes. J. Biol. Chem., (1995) 270, 25702-25708.
^{xxxi} Hampel, Arnold; Tritz, Richard; Hicks, Margaret; Cruz, Phillip. ‘Hairpin’ catalytic RNA model: evidence for helixes and sequence requirement for substrate RNA. Nucleic Acids Res. (1990), 18(2), 299-304.
^{xxxii} Chowrira, Bharat M.; Berzal-Herranz, Alfredo; Burke, John M.. Novel guanosine requirement for catalysis by the hairpin ribozyme. Nature (London) (1991), 354(6351), 320-2.
^{xxxiii} Berzal-Herranz, Alfredo; Joseph, Simpson; Chowrira, Bharat M.; Butcher, Samuel E.; Burke, John M.. Essential nucleotide sequences and secondary structure elements of the hairpin ribozyme. EMBO J. (1993), 12(6), 2567-73.
^{xxxiv} Joseph, Simpson; Berzal-Herranz, Alfredo; Chowrira, Bharat M.; Butcher, Samuel E.. Substrate selection rules for the hairpin ribozyme determined by in vitro selection, mutation, and analysis of mismatched substrates. Genes Dev. (1993), 7(1), 130-8.
^{xxxv} Berzal-Herranz, Alfredo; Joseph, Simpson; Burke, John M.. In vitro selection of active hairpin ribozymes by sequential RNA-catalyzed cleavage and ligation reactions. Genes Dev. (1992), 6(1), 129-34.
^{xxxvi} Hegg, Lisa A.; Fedor, Martha J.. Kinetics and Thermodynamics of Intermolecular Catalysis by Hairpin Ribozymes. Biochemistry (1995), 34(48), 15813-28.
^{xxxvii} Grasby, Jane A.; Mersmann, Karin; Singh, Mohinder; Gait, Michael J.. Purine Functional Groups in Essential Residues of the Hairpin Ribozyme Required for Catalytic Cleavage of RNA. Biochemistry (1995), 34(12), 4068-76.
^{xxxviii} Schmidt, Sabine; Beigelman, Leonid; Karpeisky, Alexander; Usman, Nassim; Sorensen, Ulrik S.; Gait, Michael J.. Base and sugar requirements for RNA cleavage of essential nucleoside residues in internal loop B of the hairpin ribozyme: implications for secondary structure. Nucleic Acids Res. (1996), 24(4), 573-81.
^{xxxix} Perrotta, Anne T.; Been, Michael D.. Cleavage of oligoribonucleotides by a ribozyme derived from the hepatitis .delta. virus RNA sequence. Biochemistry (1992), 31(1), 16-21.
^{xl} Perrotta, Anne T.; Been, Michael D.. A pseudoknot-like structure required for efficient self-cleavage of hepatitis delta virus RNA. Nature (London) (1991), 350(6317), 434-6.
^{xli} Puttaraju, M.; Perrotta, Anne T.; Been, Michael D.. A circular trans-acting hepatitis delta virus ribozyme. Nucleic Acids Res. (1993), 21(18), 4253-8.

TABLE II

| | | | | | |
|---|---|---|---|---|---|
| Reagent | Equivalents | Amount | Wait Time* DNA | Wait Time* 2′-O-methyl | Wait Time*RNA |

A. 2.5 μmol Synthesis Cycle ABI 394 Instrument

| | | | | | | |
|---|---|---|---|---|---|---|
| Phosphoramidites | 6.5 | 163 | μL | 45 sec | 2.5 min | 7.5 min |
| S-Ethyl Tetrazole | 23.8 | 238 | μL | 45 sec | 2.5 min | 7.5 min |
| Acetic Anhydride | 100 | 233 | μL | 5 sec | 5 sec | 5 sec |
| N-Methyl | 186 | 233 | μL | 5 sec | 5 sec | 5 sec |
| Imidazole | | | | | | |
| TCA | 176 | 2.3 | mL | 21 sec | 21 sec | 21 sec |
| Iodine | 11.2 | 1.7 | mL | 45 sec | 45 sec | 45 sec |
| Beaucage | 12.9 | 645 | μL | 100 sec | 300 sec | 300 sec |
| Acetonitrile | NA | 6.67 | μL | NA | NA | NA |

B. 0.2 μmol Synthesis Cycle ABI 394 Instrument

| | | | | | | |
|---|---|---|---|---|---|---|
| Phosphoramidites | 15 | 31 | μL | 45 sec | 233 sec | 465 sec |
| S-Ethyl Tetrazole | 38.7 | 31 | μL | 45 sec | 233 min | 465 sec |
| Acetic Anhydride | 655 | 124 | μL | 5 sec | 5 sec | 5 sec |
| N-Methyl | 1245 | 124 | μL | 5 sec | 5 sec | 5 sec |
| Imidazole | | | | | | |
| TCA | 700 | 732 | μL | 10 sec | 10 sec | 10 sec |
| Iodine | 20.6 | 244 | μL | 15 sec | 15 sec | 15 sec |
| Beaucage | 7.7 | 232 | μL | 100 sec | 300 sec | 300 sec |
| Acetonitrile | NA | 2.64 | mL | NA | NA | NA |

C. 0.2 μmol Synthesis Cycle 96 well Instrument

| | Equivalents:DNA/ | Amount: DNA/2′-O- | | | Wait Time* 2′-O- | |
|---|---|---|---|---|---|---|
| Reagent | 2′-O-methyl/Ribo | methyl/Ribo | | Wait Time* DNA | methyl | Wait Time* Ribo |
| Phosphoramidites | 22/33/66 | 40/60/120 | μL | 60 sec | 180 sec | 360 sec |
| S-Ethyl Tetrazole | 70/105/210 | 40/60/120 | μL | 60 sec | 180 min | 360 sec |
| Acetic Anhydride | 265/265/265 | 50/50/50 | μL | 10 sec | 10 sec | 10 sec |
| N-Methyl | 502/502/502 | 50/50/50 | μL | 10 sec | 10 sec | 10 sec |
| Imidazole | | | | | | |
| TCA | 238/475/475 | 250/500/500 | μL | 15 sec | 15 sec | 15 sec |
| Iodine | 6.8/6.8/6.8 | 80/80/80 | μL | 30 sec | 30 sec | 30 sec |
| Beaucage | 34/51/51 | 80/120/120 | | 100 sec | 200 sec | 200 sec |
| Acetonitrile | NA | 1150/1150/1150 | μL | NA | NA | NA |

*Wait time does not include contact time during delivery.

TABLE III
Nucleic acid sequences

| | | | |
|---|---|---|---|
| | | | Seq.ID |
| RPI# | Name | Sequence | No. |

| | | | |
|---|---|---|---|
| 15404 | R-2.1 & 2.7 | AAGCACUAAUGGAGA | 1 |
| 17161 | R-3.1 | AAGCACUAACAGUAA | 2 |
| 15400 | SM-2.1 | UCUCCAU CUGAUGAGGCCGUUAGGCCGAA AGUGCUUG | 3 |
| 17159 | SM-2.7 | UCUCCAU CUGAUGAGGCCGUUAGGCCGAA AGUGCUUG CGAGUG | 4 |
| 17160 | SM-3.1 | UUACUGU CUGAUGAGGCCGUUAGGCCGAA AGUGCUUG CGAGUG | 5 |
| 17162 | s-2.1 | caagcacuuucucaucagauggaga | 6 |
| 17163 | s-2.2 | cacucgcaagcacuuucucaucagauggaga | 7 |
| 17164 | s-2.3 | cacucgcaagcacccuaucaggcagua | 8 |
| 17165 | s-2.4 | cacucgcaagcacccuaucagguggaga | 9 |
| 15405 | T-2a | UACUGCCUGAUAGGGUGCUUGCGAGUG | 10 |

UPPER CASE = RIBO
lower case = 2′-O-methyl

1. A nucleic acid sensor molecule comprising an enzymatic nucleic acid component and one or more sensor components, wherein, in response to an interaction of a single stranded RNA (ssRNA) having a single nucleotide polymorphism (SNP) with the nucleic acid sensor molecule in a system, the enzymatic nucleic acid component catalyzes a chemical reaction resulting in a detectable response.

2. A nucleic acid sensor molecule comprising an enzymatic nucleic acid component and one or more sensor components, wherein, in response to an interaction of a single stranded DNA (ssDNA) having a SNP with the nucleic acid sensor molecule in a system, the enzymatic nucleic acid component catalyzes a chemical reaction resulting in a detectable response.

3. A nucleic acid sensor molecule comprising an enzymatic nucleic acid sensor molecule in a system, and one or more sensor components, wherein, in response to an interaction of a single stranded RNA (ssRNA) with the nucleic acid sensor molecule in a system, the enzymatic nucleic acid component catalyzes a chemical reaction resulting in cleavage of a predetermined RNA molecule associated with a disease.

4. A nucleic acid sensor molecule comprising an enzymatic nucleic acid component and one or more sensor components, wherein, in response to an interaction of a single stranded DNA (ssDNA) with the nucleic acid sensor molecule in a system, the enzymatic nucleic acid component catalyzes a chemical reaction resulting in cleavage of a predetermined RNA molecule associated with a disease.

5. A nucleic acid sensor molecule comprising an enzymatic nucleic acid component and one or more sensor components, wherein, in response to an interaction of a peptide with the nucleic acid sensor molecule in a system, the enzymatic nucleic acid component catalyzes a chemical reaction resulting in cleavage of a peptide with the nucleic acid sensor molecule in a system, the enzymatic nucleic acid component catalyzes a chemical reaction resulting in cleavage of a predetermined RNA molecule associated with a disease.

6. A nucleic acid sensor molecule comprising an enzymatic nucleic acid component and one or more sensor components, wherein, in response to an interaction of a protein with the nucleic acid sensor molecule in a system, the enzymatic nucleic acid component catalyzes a chemical reaction resulting in cleavage of a predetermined RNA molecule associated with a disease.

7. A nucleic acid sensor molecule comprising an enzymatic nucleic acid component and one or more sensor components, wherein, in response to an interaction of a single stranded RNA (ssRNA) having a SNP with the nucleic acid sensor molecule in a system, the enzymatic nucleic acid component catalyzes a chemical reaction resulting in ligation of a predetermined RNA molecule to another predetermined RNA molecule.

8. A nucleic acid sensor molecule comprising an enzymatic nucleic acid component and one or more sensor components, wherein, in response to an interaction of a single stranded DNA (ssDNA) having a SNP with the nucleic acid sensor molecule in a system, the enzymatic nucleic acid component catalyzes a chemical reaction resulting in ligation of a predetermined RNA molecule to another predetermined RNA molecule.

9. A method comprising:
a. contacting the nucleic acid sensor molecule of claim 1 with a system comprising at least one ssRNA having a SNP under conditions suitable for the enzymatic nucleic acid component of the nucleic acid sensor molecule to catalyzes a chemical reaction resulting in a detectable response; and
b. assaying for the chemical reaction resulting in a detectable response.

10. A method comprising:
a. contacting the nucleic acid sensor molecule of claim 2 with a system comprising at least one ssDNA having a SNP under conditions suitable for the enzymatic nucleic acid component of the nucleic acid sensor molecule to catalyzes a chemical reaction resulting in a detectable response; and
b. assaying for the chemical reaction resulting in a detectable response.

11. A method comprising contacting the nucleic acid sensor molecule of claim 3 with a system comprising at least one ssRNA under conditions suitable for the enzymatic nucleic acid component of the nucleic acid sensor molecule to cleave the predetermined RNA molecule.

12. A method comprising contacting the nucleic acid sensor molecule of claim 4 with a system comprising at least one ssDNA under conditions suitable for the enzymatic nucleic acid component of the nucleic acid sensor molecule to cleave the predetermined RNA molecule

13. A method comprising contacting the nucleic acid sensor molecule of claim 5 with a system comprising at least one peptide under conditions suitable for the enzymatic nucleic acid component of the nucleic acid sensor molecule to cleave the predetermined RNA molecule.

14. A method comprising contacting the nucleic acid sensor molecule of claim 6 with a system comprising at least one protein, under conditions suitable for the enzymatic nucleic acid component of the nucleic acid sensor molecule to cleave the predetermined RNA molecule.

15. A method comprising contacting the nucleic acid sensor molecule of claim 7 with a system comprising at least one ssRNA having a SNP under conditions suitable for the enzymatic nucleic acid component of the nucleic acid sensor molecule to ligate a predetermined RNA molecule to another predetermined RNA molecule.

16. A method comprising contacting the nucleic acid sensor molecule of claim 8 with a system comprising at least one ssDNA having a SNP under conditions suitable for the enzymatic nucleic acid component of the nucleic acid sensor molecule to ligate a predetermined RNA molecule to another predetermined RNA molecule.

17. The nucleic acid sensor molecule of any of claim 1 or claim 2, wherein said chemical reaction is cleavage of a phosphodiester internucleotide linkage.

18. The nucleic acid sensor molecule of any of claim 1 or claim 2, wherein said chemical reaction is ligation of a predetermined nucleic acid molecule to the nucleic acid sensor molecule.

19. The nucleic acid sensor molecule of claim 1 or claim 2, wherein said chemical reaction is ligation of a predetermined nucleic acid molecule to another predetermined nucleic acid molecule.

20. The nucleic acid sensor molecule of claim 1 or claim 2, wherein said chemical reaction is isomerization.

21. The nucleic acid sensor molecule of claim 1 or claim 2, wherein said chemical reaction is phosphorylation of a peptide or protein.

22. The nucleic acid sensor molecule of claim 1 or claim 2, wherein said chemical reaction is dephosphorylation of a peptide or protein.

23. The nucleic acid sensor molecule of claim 1 or claim 2, wherein said chemical reaction is RNA polymerase activity.

24. The nucleic acid molecule of claim 1 or claim 2, wherein said detectable response is an increase or decrease in fluorescence.

25. The nucleic acid molecule of claim 1 or claim 2, wherein said detectable response is an increase or decrease in enzymatic activity.

26. The nucleic acid molecule of claim 1 or claim 2, wherein said detectable response is an increase or decrease in the production of a precipitate.

27. The nucleic acid molecule of claim 1 or claim 2, wherein said detectable response is an increase or decrease in chemoluminescence.

28. The nucleic acid molecule of claim 1 or claim 2, wherein said detectable response is an increase or decrease in radioactive emission.

29. A kit comprising the nucleic acid sensor molecule of claim 1.

30. A kit comprising the nucleic acid sensor molecule of claim 2.

31. A kit comprising the nucleic acid sensor molecule of claim 3.

32. A kit comprising the nucleic acid sensor molecule of claim 4.

33. A kit comprising the nucleic acid sensor molecule of claim 5.

34. A kit comprising the nucleic acid sensor molecule of claim 6.

35. A kit comprising the nucleic acid sensor molecule of claim 7.

36. A kit comprising the nucleic acid sensor molecule of claim 8.

37. The nucleic acid sensor molecule of claim 5, wherein said peptide is an Hepatitis C Virus (HCV) peptide.

38. The nucleic acid sensor molecule of claim 6, wherein said protein is an HCV protein.

39. The nucleic acid sensor molecule of claim 5 or claim 6, wherein said sensor component is a sequence derived from the HCV 5′-UTR.

40. The nucleic acid sensor molecule of claim 39, wherein said sequence derived from the HCV 5′-UTR is structural domain IIIa.

41. The nucleic acid sensor molecule of claim 39, wherein said sequence derived from the HCV 5′-UTR is structural domain IIIb.

42. The nucleic acid sensor molecule of claim 39, wherein said sequence derived from the HCV 5′-UTR is structural domain IIIc.

43. The nucleic acid sensor molecule of claim 39, wherein said sequence derived from the HCV 5′-UTR is structural domain IIId.

44. The nucleic acid sensor molecule of claim 39, wherein said sequence derived from the HCV 5′-UTR is structural domain IIIe.

45. The nucleic acid sensor molecule of claim 39, wherein said sequence derived from the HCV 5′-UTR is structural domain IIIf.

46. The nucleic acid sensor molecule of claim 39, wherein said sequence derived from the HCV 5′-UTR is structural domain I.

47. The nucleic acid sensor molecule of claim 39, wherein said sequence derived from the HCV 5′-UTR is structural domain II.

48. The nucleic acid sensor molecule of claim 39, wherein said sequence derived from the HCV 5′-UTR is structural domain IV.

49. The nucleic acid sensor molecule of claim 5, wherein said peptide is a viral peptide.

50. The nucleic acid sensor molecule of claim 49, wherein said viral peptide is derived from HCV, Hepatitis B Virus (HBV), Human Immunodeficiency Virus (HIV), Human Papilloma Virus (HPV), Human T-cell Lyphotrophic Virus Type 1 (HTLV-1), Cytomegalovirus (CMV), Herpes Simplex Virus (HSV), respiratory syncytial virus (RSV), Rhinovirus, West Nile Virus (WNV), Hantavirus, Ebola virus, or Encephalovirus.

51. The nucleic acid sensor molecule of claim 6, wherein said protein is a viral protein.

52. The nucleic acid sensor molecule of claim 51, wherein said viral protein is derived from HCV, HBV, HIV, HPV, HTLV-1, CMV, HSV, RSV, Rhinovirus, WNV, Hantavirus, Ebola virus, or Encephalovirus.

53. The nucleic acid sensor molecule of claim 37 or claim 38, wherein said predetermined RNA is associated with HCV infection.

54. The nucleic acid sensor molecule of claim 39, wherein said predetermined RNA is associated with HCV infection.


SEQUENCE LISTING (49 sequences)

SEQ ID NO: 1 - RNA, 15 nt, Artificial Sequence
  Description of Artificial Sequence  Synthetic reporter sequence
aagcacuaauggaga

SEQ ID NO: 2 - RNA, 15 nt, Artificial Sequence
  Description of Artificial Sequence  Synthetic reporter sequence
aagcacuaacaguaa

SEQ ID NO: 3 - RNA, 37 nt, Artificial Sequence
  Description of Artificial Sequence  Synthetic sensor sequence
ucuccaucugaugaggccguuaggccgaaagugcuug

SEQ ID NO: 4 - RNA, 43 nt, Artificial Sequence
  Description of Artificial Sequence  Synthetic sensor sequence
ucuccaucugaugaggccguuaggccgaaagugcuugcgagug

SEQ ID NO: 5 - RNA, 43 nt, Artificial Sequence
  Description of Artificial Sequence  Synthetic sensor sequence
uuacugucugaugaggccguuaggccgaaagugcuugcgagug

SEQ ID NO: 6 - RNA, 25 nt, Artificial Sequence
  Description of Artificial Sequence  Synthetic sensor component sequence
caagcacuuucucaucagauggaga

SEQ ID NO: 7 - RNA, 31 nt, Artificial Sequence
  Description of Artificial Sequence  Synthetic sensor component sequence
cacucgcaagcacuuucucaucagauggaga

SEQ ID NO: 8 - RNA, 26 nt, Artificial Sequence
  Description of Artificial Sequence  Synthetic sensor component sequence
cacucgcaagcaccuaucaggcagua

SEQ ID NO: 9 - RNA, 28 nt, Artificial Sequence
  Description of Artificial Sequence  Synthetic sensor component sequence
cacucgcaagcacccuaucagguggaga

SEQ ID NO: 10 - RNA, 27 nt, Artificial Sequence
  Description of Artificial Sequence  Synthetic target signaling sequence
uacugccugauagggugcuugcgagug

SEQ ID NO: 11 - RNA, 15 nt, Artificial Sequence
  Description of Artificial Sequence  Generic target sequence
nnnnnnuhnnnnnnn

SEQ ID NO: 12 - RNA, 36 nt, Artificial Sequence
  Description of Artificial Sequence  Enzymatic Nucleic Acid
nnnnnnncugaugagnnngaaannncgaaannnnnn

SEQ ID NO: 13 - RNA, 14 nt, Artificial Sequence
  Description of Artificial Sequence  Generic target sequence
nnnnnchnnnnnnn

SEQ ID NO: 14 - RNA, 35 nt, Artificial Sequence
  Description of Artificial Sequence  Enzymatic Nucleic Acid
nnnnnnncugaugagnnngaaannncgaannnnnn

SEQ ID NO: 15 - RNA, 15 nt, Artificial Sequence
  Description of Artificial Sequence  Generic target sequence
nnnnnnygnnnnnnn

SEQ ID NO: 16 - RNA, 35 nt, Artificial Sequence
  Description of Artificial Sequence  Enzymatic Nucleic Acid Motif
nnnnnnnugauggcaugcacuaugcgcgnnnnnnn

SEQ ID NO: 17 - RNA, 48 nt, Artificial Sequence
  Description of Artificial Sequence  Enzymatic Nucleic Acid Motif
gugugcaaccggaggaaacucccuucaaggacgaaaguccgggacggg

SEQ ID NO: 18 - RNA, 16 nt, Artificial Sequence
  Description of Artificial Sequence  Target sequence for SEQ ID NO 17
gccguggguugcacac

SEQ ID NO: 19 - RNA, 36 nt, Artificial Sequence
  Description of Artificial Sequence  Enzymatic Nucleic Acid Motif
gugccuggccgaaaggcgagugaggucugccgcgcn

SEQ ID NO: 20 - RNA, 15 nt, Artificial Sequence
  Description of Artificial Sequence  Target sequence for SEQ ID NO 19
gcgcggcgcaggcac

SEQ ID NO: 21 - DNA, 16 bp, Artificial Sequence
  Description of Artificial Sequence  Enzymatic Nucleic Acid Motif
rggctagctacaacga

SEQ ID NO: 22 - RNA, 12 nt, Artificial Sequence
  Description of Artificial Sequence  Generic Reporter Molecule
nnnnnygnnnnn

SEQ ID NO: 23 - RNA, 35 nt, Artificial Sequence
  Description of Artificial Sequence  Synthetic Sensor Molecule Motif
nnnnnngnnnnnnnnnncgagugaggucurnnnnn

SEQ ID NO: 24 - RNA, 23 nt, Artificial Sequence
  Description of Artificial Sequence  Synthetic Sensor Molecule Motif
nnnnncgagugaggucurnnnnn

SEQ ID NO: 25 - RNA, 11 nt, Artificial Sequence
  Description of Artificial Sequence  Synthetic Target Signaling Molecule
nnnnrgnnnnn

SEQ ID NO: 26 - RNA, 21 nt, Hepatitis C virus
gguccuuucuuggauaaaccc

SEQ ID NO: 27 - RNA, 42 nt, Artificial Sequence
  Description of Artificial Sequence  Synthetic Sensor/Reporter Molecule
ggguuuaucgagugaggucurnnnnnnnnnnnnnnygcaaga

SEQ ID NO: 28 - RNA, 54 nt, Artificial Sequence
  Description of Artificial Sequence  Synthetic Sensor Molecule
nnnnnngagccgaguagcguugggucgcgaaaggcucgagugaggucurnnnnn

SEQ ID NO: 29 - RNA, 72 nt, Artificial Sequence
  Description of Artificial Sequence  Synthetic Sensor Molecule
nnnnnngnnnnnnnnnagccgaguagcguugggucgcgaaaggcunnnnnnnnncgagugaggucurnnnnn

SEQ ID NO: 30 - RNA, 39 nt, Artificial Sequence
  Description of Artificial Sequence  Synthetic Sensor Molecule
nnnnnngnnnnnnnnnncgagugaggucurnnnnnnnnn

SEQ ID NO: 31 - RNA, 10 nt, Artificial Sequence
  Description of Artificial Sequence  Generic Target Molecule
nnnnnngnnn

SEQ ID NO: 32 - RNA, 39 nt, Artificial Sequence
  Description of Artificial Sequence  Synthetic Reporter/Sensor Molecule
nnnnncgagugaggucurnnnnnnnnnnnnnnygnnnnn

SEQ ID NO: 33 - RNA, 34 nt, Artificial Sequence
  Description of Artificial Sequence  Synthetic Reporter/Sensor Molecule
nnnnncgagugaggucurnnnnnnnnnnnnnnyg

SEQ ID NO: 34 - RNA, 27 nt, Artificial Sequence
  Description of Artificial Sequence  Target Molecule
cggguccuuucuuggauaaacccgcuc

SEQ ID NO: 35 - RNA, 47 nt, Artificial Sequence
  Description of Artificial Sequence  Synthetic Sensor Molecule
gagcggguuuauccaagaaaggaccuuuucgagugaggucugacggc

SEQ ID NO: 36 - RNA, 12 nt, Artificial Sequence
  Description of Artificial Sequence  Synthetic Reporter Molecule
gccgucguugga

SEQ ID NO: 37 - RNA, 21 nt, Artificial Sequence
  Description of Artificial Sequence  Target Sequence
gguccuuucuuggauaaaccc

SEQ ID NO: 38 - RNA, 12 nt, Artificial Sequence
  Description of Artificial Sequence  Synthetic Reporter Molecule
gccgucguuauu

SEQ ID NO: 39 - DNA, 21 bp, Artificial Sequence
  Description of Artificial Sequence  Synthetic Sensor Molecule
gggcttatccaagaaaggacc

SEQ ID NO: 40 - RNA, 37 nt, Artificial Sequence
  Description of Artificial Sequence  Erk Modulated Sensor Molecule
gcgugaccugaugaggccgaaaggccgaaacguuccc

SEQ ID NO: 41 - RNA, 41 nt, Artificial Sequence
  Description of Artificial Sequence  Erk Modulated Sensor Component
ggauaaggaggauuuccgaaagcggcuacgguccgccaucc

SEQ ID NO: 42 - RNA, 74 nt, Artificial Sequence
  Description of Artificial Sequence  Erk Modulated Sensor Molecule
gcgugaccugaugagucacgcuaaggaggauuuccgaaagcggcuacgguccgccaguguuacgaaacguuccc

SEQ ID NO: 43 - RNA, 38 nt, Artificial Sequence
  Description of Artificial Sequence  Half- Zinzyme
uuaucgagugaggucugacggcgccgucgcaagaaagg

SEQ ID NO: 44 - RNA, 17 nt, Artificial Sequence
  Description of Artificial Sequence  Target Signaling Molecule
gguccuuucuuggauaa

SEQ ID NO: 45 - RNA, 17 nt, Hepatitis C virus
ccuuucuuggauaaaug

SEQ ID NO: 46 - RNA, 112 nt, Artificial Sequence
  Description of Artificial Sequence  Sensor Ligase Molecule
aaaccagucggaacacuauacgacugguaccguaaaagacaaaugugcccucagagcaaggaccgaucuucggauccaggggaggcaccccccgguggcuuuaacgccaacg

SEQ ID NO: 47 - RNA, 17 nt, Hepatitis C virus
gguccuuucuuggauaa

SEQ ID NO: 48 - RNA, 366 nt, Hepatitis C virus
gccagcccccgauugggggcgacacuccaccauagaucacuccccugugaggaacuacugucuucacgcagaaagcgucuagccauggcguuaguaugagugucgugcagccuccaggacccccccucccgggagagccauaguggucugcggaaccggugaguacaccggaauugccaggacgaccggguccuuucuuggaucaacccgcucaaugccuggagauuugggcgugcccccgcgagacugcuagccgaguaguguugggucgcgaaaggccuugugguacugccugauagggugcuugcgagugccccgggaggucucguagaccgugcaccaugagcacgaauccuaaaccucaaa

SEQ ID NO: 49 - RNA, 12 nt, Artificial Sequence
  Description of Artificial Sequence  Generic Target Sequence
nnnnnygynnnn